(19) United States
(12) Patent Application Publication
(10) Pub. No.: US 2020/0308144 A1
(43) Pub. Date: Oct. 1, 2020

(54) Multimeric piperidine derivatives

(71) Applicant: Bellicum Pharmaceuticals, Inc., Houston, TX (US)

(72) Inventors: Steven Toler, Houston, TX (US); Slawomir Szymanski, Spring, TX (US)

(21) Appl. No.: 16/955,693

(22) PCT Filed: Dec. 19, 2018
(86) PCT No.: PCT/US2018/066532

§ 371 Date: Jun. 18, 2020

Related U.S. Application Data
(60) Provisional application No. 62/608,552, filed on Dec. 20, 2017.

(51) Int. Cl. C07D 401/12

(52) U.S. Cl. CPC C07D 401/12

(57) ABSTRACT

The technology relates in part to compounds that bind to multimeric ligand binding regions, referred to herein as “multimeric compounds.” In certain examples, the multimeric compounds provided herein bind to and multimerize polypeptides that bind to rimiducid, such as for example, chimeric polypeptides that comprise FKBP12 polypeptide variant regions. Multimeric compounds provided include those having a structure of Formula I. Where A, Z, Y, Z′ and A′ moieties are described herein.

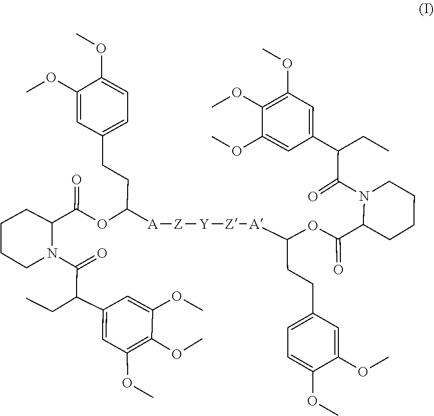

### RELATED APPLICATIONS

Priority is claimed to U.S. Provisional Patent Application Ser. No. 62/608,552, filed Dec. 20, 2018, by Steven Toler and Slawomir Szymanski, entitled “Multimeric Compounds,” which is referred to and incorporated by reference thereof, in its entirety.

### FIELD

The technology relates in part to compounds that bind to multimeric ligand binding regions, referred to herein as “multimeric compounds.” In certain examples, the multimeric compounds bind to and multimerize polypeptides that bind to rimiducid, such as for example, chimeric polypeptides that comprise FKBP12 polypeptide variant regions.

### BACKGROUND

Chemical induction of polypeptide dimerization with small molecules can be used to switch protein function and alter cell physiology. A high specificity, efficient dimerizer is rimiducid (AP1903), a multimeric ligand that has two identical, protein-binding surfaces arranged tail-to-tail. Each of the protein-binding surfaces of rimiducid are capable of binding with high affinity and specificity to a mutant or variant of the FK506-binding protein FKBP12. Attachment of one or more FKBP12 mutant domains onto one or more cell signaling molecules that normally rely on homodimerization can convert that protein to rimiducid control. Dimerization or multimerization with rimiducid may be used, for example, in the context of an inducible cell killing safety switch, and/or an inducible cell activating switch, for cellular therapy, where costimulatory polypeptides are used to stimulate immune activity.

### SUMMARY

Provided herein are compounds that can be used to multimerize polypeptides that comprise multimeric ligand binding regions, which are referred to herein as “multimeric compounds.” In some embodiments, a multimeric compound binds to multimeric ligand binding regions of a polypeptide, such as, for example, variants of the FK506 binding protein FKBP12. In some embodiments, a multimeric compound binds to a FKBP12 polypeptide variant that also binds to rimiducid, such as a FKBP12 polypeptide variant that has an amino acid substitution at residue 36. In certain embodiments, multimeric compounds provided herein have greater solubility than rimiducid in water and in other pharmaceutically acceptable aqueous solutions.

In certain aspects, provided herein are compounds having a structure of the following Formula I:

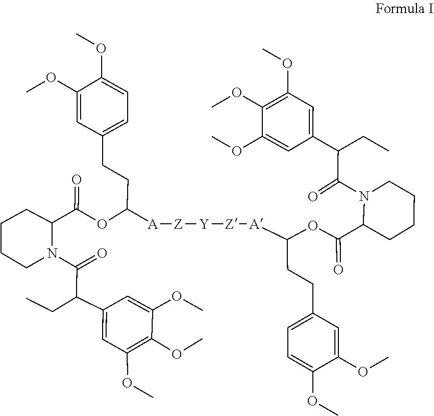

or a pharmaceutically acceptable salt thereof, wherein:
Z and Z′ are the same or different and each independently is O, NR¹², —N═, S, SO, SO₂ or CH₂;
Y is L, M or Q:

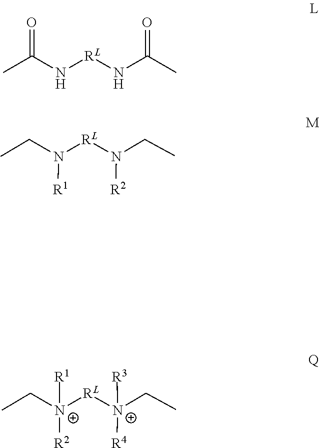

R¹, R², R³, and R⁴ are the same or different, and each is independently hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when Y is M, R¹ and R² together with —N—R_{L}—N— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy; when Y is Q, R¹ and R² together with N⁺ may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when Y is Q, R³ and R⁴ together with N⁺ may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, heterocycle-alkyl, cycloalkylalkyl, cycloalkyl, heterocycloalkyl, heteroaryl, heteroarylalkyl, alkylsulfonyl, sulfonamide, alkylsulfonamido and alkylsilyloxy;
when Y is Q, R¹ and R³ together with —N⁺—R_{L}—N⁺— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, heterocycle-alkyl, cycloalkylalkyl, cycloalkyl, heterocycloalkyl, heteroaryl, heteroarylalkyl, alkylsulfonyl, sulfonamide, alkylsulfonamido and alkylsilyloxy;
when Y is Q, R² and R⁴ together with —N⁺—R_{L}—N⁺— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, heterocycle-alkyl, cycloalkylalkyl, cycloalkyl, heterocycloalkyl, heteroaryl, heteroarylalkyl, alkylsulfonyl, sulfonamide, alkylsulfonamido and alkylsilyloxy;
Optionally, when Y is Q, one of the groups: R¹, R², R³ and R⁴ may be nonexistent. If one of the groups: R¹, R², R³ or R⁴ is non-existent, and Y is Q, the compound is a monosalt;
R_{L} is a lower alkylene, alkenylene, alkynylene, acyl, cycloalkyl, or aryl, in which none or one or more carbon atoms are replaced by O, NR¹³, S, SO, SO₂, and which is optionally substituted with hydroxyl, alkoxyl, amino, alkylamino, thiol, thioalkyl, or halogen;
A and A′ are the same or different and each independently are

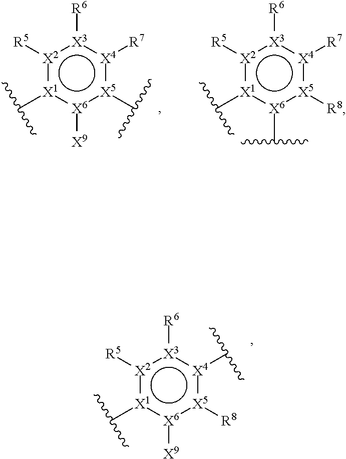

thiophene, furan, pyrrole, carbonyl, lower dialkyl ether, lower dialkyl thioether, lower dialkylamino, cyclopropylene, alkanylene, cycloalkanylene, alkenylene, cycloalkenylene, lower alkynylene, lower cycloalkynylene, carbamate, sulfanyl, sulfinyl, sulfonyl, thiocarbonyl, imino, or hydroxyimino, in which independently none or one or more carbon atoms are replaced by O, NR¹⁴, S, SO, SO₂, and which is optionally substituted with hydroxyl, alkoxyl, amino, alkylamino, thiol, thioalkyl, or halogen;
R¹² is hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, heterocycle-alkyl, cycloalkylalkyl, cycloalkyl, heterocycloalkyl, heteroaryl, heteroarylalkyl, alkylsulfonyl, sulfonamide, alkylsulfonamido and alkylsilyloxy;
R¹³ is hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, heterocycle-alkyl, cycloalkylalkyl, cycloalkyl, heterocycloalkyl, heteroaryl, heteroarylalkyl, alkylsulfonyl, sulfonamide, alkylsulfonamido and alkylsilyloxy;
R¹⁴ hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, heterocycle-alkyl, cycloalkylalkyl, cycloalkyl, heterocycloalkyl, heteroaryl, heteroarylalkyl, alkylsulfonyl, sulfonamide, alkylsulfonamido and alkylsilyloxy;
X¹, X², X³, X⁴, X⁵ and X⁶ independently are carbon or nitrogen with the proviso that none, one, two or three of X¹, X², X³, X⁴, X⁵ and X⁶ are nitrogen;
when X², X³, X⁴, X⁵ or X⁶ is carbon, R⁵, R⁶, R⁷, R⁸ or R⁹, respectively, independently is hydrogen, hydroxyl, halogen, C1-C2 alkyl or C1-C2 alkyl substituted with hydroxyl, halogen or NR¹⁰R¹¹;
R¹⁰ and R¹¹ independently are hydrogen or C1-C2 alkyl;
when X², X³, X⁴ or X⁶ is nitrogen, R⁵, R⁶, R⁷, R⁸ or R⁹, respectively, is not present or is hydrogen, C1-C2 alkyl or C1-C2 alkyl substituted with hydroxyl, halogen or NR¹⁰R¹¹;
with the proviso that (i) Y is M or Q when A and A′ are phenyl and Z and Z′ are oxygen, or (ii) A and A′ are not the same, or one or both of A and A′ are not phenyl, or Z and Z′ are not the same, or one or both of Z and Z′ are not oxygen, when Y is L and R_{L} is —CH₂—CH₂—.
Compounds of Formula I are referred to herein as “multimeric compounds.”

A multimeric compound may also be referred to herein as a multimerizing agent, a multimerizing compound, a multimerizing ligand, a multimeric agent, a multimeric compound, or a multimeric ligand. The term “multimerize” or multimerization refers to the dimerization of two polypeptides, or the multimerization of more than two polypeptides.

Certain embodiments are described further in the following description, examples, claims and drawings.

### INCORPORATION BY REFERENCE

All publications, patents and patent applications, GENBANK sequences (e.g., available at the World Wide Web Uniform Resource Locator (URL) ncbi.nlm.nih.gov of the National Center for Biotechnology Information (NCBI)), sequences available through other databases, and websites and other published materials referred to throughout the entire disclosure herein, unless noted otherwise, are incorporated by reference in their entirety. Citation of any publications, patents and patent applications, GENBANK (and other database) sequences, websites and other published materials herein is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 provides an example of a scheme (Scheme 1) for the synthesis of Compound A.

FIG. 2 provides an example of a scheme (Scheme 2) for the synthesis of Compound A.

FIG. 3 provides a chemical structure of Compound A (C₈₀H₁₀₆N4O₁₈), which has a molecular weight of 1412 grams per mole (g/mol).

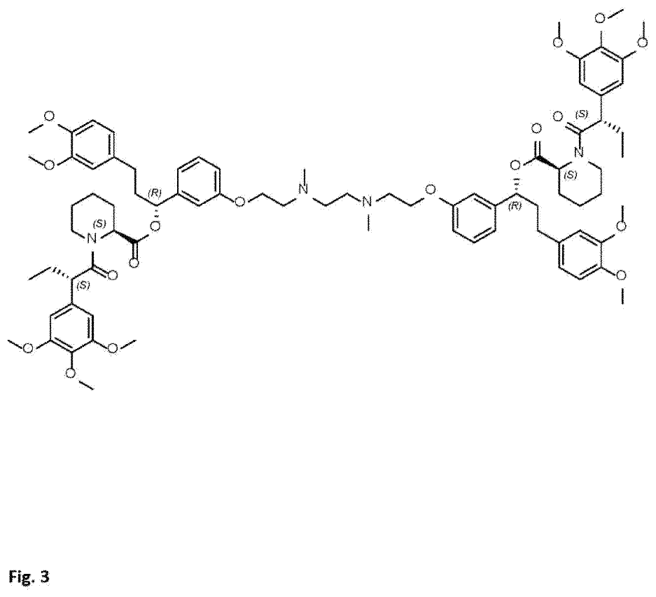

FIG. 4 provides Liquid Chromatography-Mass Spectrometry (LCMS) verification graphs of Compound A., having a molecular weight of 1412 g/mol, where the counter ion for the salt is phosphate. Top to bottom as referenced on the left side (0,0.00): UV Chromatogram, 190 nm; TIC+ (total ion current); BPC+ (base peak chromatogram).

FIG. 5 provides protein NMR structural assignments for Compound A (free base), and three salts having the counter ions fumarate, phosphate (Compound A.1), and hydrochloride (Compound A.2), from top to bottom, respectively.

FIG. 6 provides a graph of dynamic vapor sorption isotherm of Compound A.1 (phosphate salt) at room temperature.

FIG. 7 is a graph of an in vitro functional binding activity assay, measured as SEAP activity, to FKBP12-F36V as provided in a chimeric inducible caspase-9 polypeptide (iC9). The in vitro functional binding of rapamycin, rimiducid, Compound A.2 (hydrochloride salt), and Compound A.1 (phosphate salt) to the inducible chimeric polypeptide was assayed, as indicated in order from top to bottom on the right side of the graph (x=100).

### DETAILED DESCRIPTION

The multimeric ligand rimiducid can be used to rapidly crosslink chimeric polypeptides that include a region that binds to the multimeric ligand, such as, for example, a multimeric ligand binding region or a multimerizing region. By attaching one or more multimerizing regions to one or more cell signaling proteins that normally rely on homodimerization the protein, or proteins can be multimerized by contact with rimiducid. For example, contacting modified cells that express chimeric polypeptides that include one or more multimerizing regions and an apoptosis inducing polypeptide with rimiducid, activates a cellular safety switch, resulting in apoptosis. Contacting modified cells that express chimeric polypeptides that include one or more multimerizing regions and one or more costimulatory polypeptides with rimiducid, activates costimulatory activity. The dimerizer rimiducid has a relatively low solubility in water of approximately 700 pM. Compounds provided herein, referred to generally as “multimeric compounds,” can exhibit favorable specificity for, and efficient dimerization of, FKBP polypeptides (e.g., FKBP12 modified forms such as FKBP12-F36V), and can be considered favorable multimeric ligands relative to rimiducid. Multimeric compounds provided herein can exhibit favorable solubility in water and other pharmaceutically acceptable aqueous solutions relative to rimiducid.

### Multimeric Compounds

Multimeric compounds described herein, or pharmaceutically acceptable salts thereof, in some embodiments bind with relatively high affinity to FKBP polypeptides, and sometimes with high binding affinity to FKBP12 polypeptide variants to which rimiducid binds with high affinity. Multimeric compounds described herein, or pharmaceutically acceptable salts thereof, sometimes exhibit greater water solubility than rimiducid. Sometimes, independent of water solubility, multimeric compounds described herein, or pharmaceutically acceptable salts thereof, exhibit about the same or better binding to a FKBP12 polypeptide variant as compared to rimiducid.

The compound named rimiducid has the following structure:

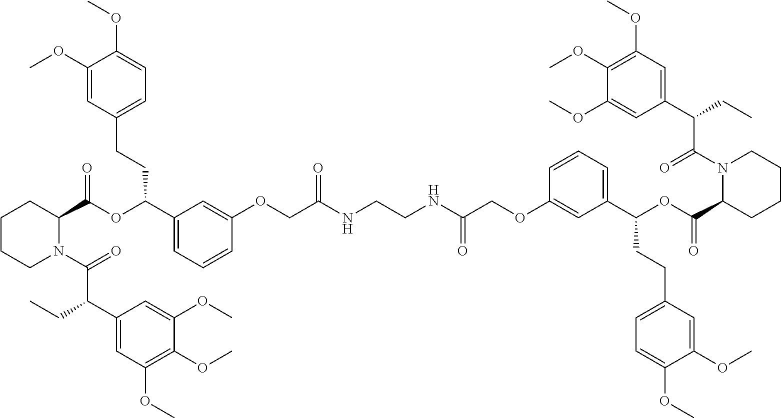 [SMILES: CC[C@H](C(=O)N1CCCC[C@H]1C(=O)O[C@H](CCc1ccc(OC)c(OC)c1)c1cccc(OCC(=O)NCCNC(=O)COc2cccc([C@@H](CCc3ccc(OC)c(OC)c3)OC(=O)[C@@H]3CCCCN3C(=O)[C@@H](CC)c3cc(OC)c(OC)c(OC)c3)c2)c1)c1cc(OC)c(OC)c(OC)c1]

Multimeric compounds described herein often have a structure of Formula I, as described in the “Summary” section above, or a pharmaceutical salt thereof. In certain embodiments, A or A′ independently is

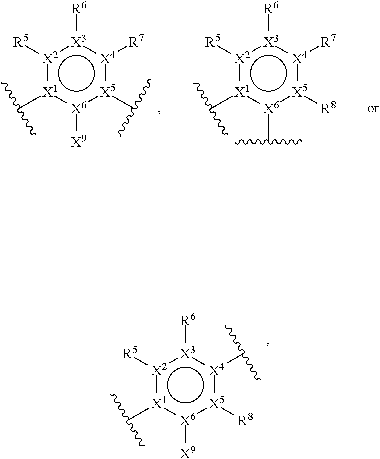

where X¹, X², X³, X⁴, X⁵ and X⁶ independently are carbon or nitrogen, provided that none, one, two or three of X¹, X², X³, X⁴, X⁵ and X⁶ are nitrogen. In certain embodiments, when X², X³, X⁴, X⁵ or X⁶ independently is carbon, R⁵, R⁶, R⁷, R⁸ or R⁹, respectively, independently is a Group A moiety chosen from hydrogen, hydroxyl, halogen, C1-C2 alkyl or C1-C2 alkyl substituted with hydroxyl, halogen or NR¹⁰R¹¹. Stated another way, when X² is carbon, R⁵ is a Group A moiety; when X³ is carbon, R⁶ is a Group A moiety; when X⁴ is carbon, R⁷ is a Group A moiety; when X⁵ is carbon, R⁸ is a Group A moiety; and when X⁶ is carbon, R⁹ is a Group A moiety. In certain embodiments, when X², X³, X⁴, X⁵ or X⁶ independently is nitrogen, R⁵, R⁶, R⁷, R⁸ or R⁹, respectively, is not present or independently is a Group B moiety chosen from hydrogen, C1-C2 alkyl or C1-C2 alkyl substituted with hydroxyl, halogen or NR¹⁰R¹¹. Stated another way, when X² is nitrogen, R⁵ is not present or is a Group B moiety; when X³ is nitrogen, R⁶ is not present or is a Group B moiety; when X⁴ is nitrogen, R⁷ is not present or is a Group B moiety; when X⁵ is nitrogen, R⁸ is not present or is a Group B moiety; and when X⁶ is nitrogen, R⁹ is not present or is a Group B moiety. In certain non-limiting embodiments, atoms at positions X¹, X², X³, X⁴, X⁵ or X⁶ in a molecule are designated in each row of the following Table 1:

TABLE 1

| X¹ | X² | X³ | X⁴ | X⁵ | X⁶ |
|---|---|---|---|---|---|
| C | C | C | C | C | C |
| N | C | C | C | C | C |
| C | N | C | C | C | C |
| C | C | N | C | C | C |
| C | C | C | N | C | C |
| C | C | C | C | N | C |
| C | C | C | C | C | N |
| N | N | C | C | C | C |
| N | C | N | C | C | C |
| N | C | C | N | C | C |
| N | C | C | C | N | C |
| N | C | C | C | C | N |
| C | N | N | C | C | C |
| C | N | C | N | C | C |
| C | N | C | C | N | C |
| C | N | C | C | C | N |
| C | C | N | N | C | C |
| C | C | N | C | N | C |
| C | C | N | C | C | N |
| C | C | C | N | N | C |
| C | C | C | N | C | N |
| C | C | C | C | N | N |
| N | N | N | C | C | C |
| N | N | C | N | C | C |
| N | N | C | C | N | C |
| N | N | C | C | C | N |
| N | C | N | N | C | C |
| N | C | N | C | N | C |
| N | C | N | C | C | N |
| N | C | C | N | N | C |
| N | C | C | N | C | N |
| N | C | C | C | N | N |
| C | N | N | N | C | C |
| C | N | N | C | N | C |
| C | N | N | C | C | N |
| C | N | C | N | N | C |
| C | N | C | N | C | N |
| C | N | C | C | N | N |
| C | C | N | N | N | C |
| C | C | N | N | C | N |
| C | C | N | C | N | N |
| C | C | C | N | N | N |

In some embodiments, the compound having Formula I, has the following Formula II:

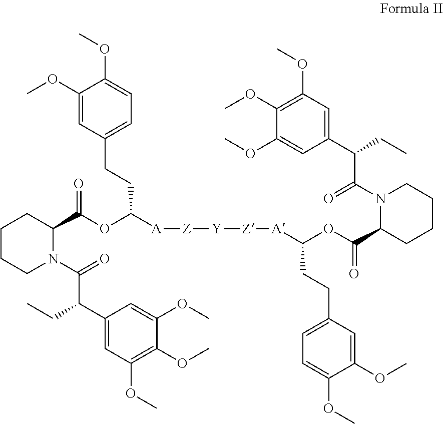

or a pharmaceutically acceptable salt thereof.

In some embodiments, where the compound is a compound of Formula I or II, R_{L} is —CH₂—CH₂—.

In some embodiments, where the compound is a compound of Formula I or II, and R_{L} is optionally CH₂—CH₂—, Y is M. In some embodiments, Y is

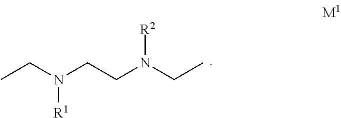

In some embodiments, where the compound is of Formula I or II, Y is M, and/or Y is M¹, and optionally R_{L} is —CH₂—CH₂—, R¹ and R² are the same.

In some embodiments, the compound is of Formula I or II, and Y is Q. In some embodiments, Q is

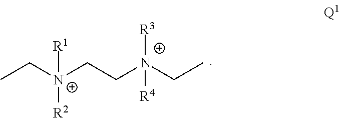

In some embodiments, where Y is Q or Q¹, R¹ and R³ are the same, and R² and R⁴ are the same.

In some embodiments, where the compound is of Formula I or II, A and A′ are the same. In some embodiments, A and A′ independently are phenyl, pyridinyl, methylpyridinyl, pyridazinyl, pyrimidinyl, pyrazinyl, triazinyl, substituted phenyl, substituted pyridinyl, substituted pyridazinyl, substituted pyrimidinyl, substituted pyrazinyl or substituted triazinyl.

In some embodiments, where the compound is of Formula I or II,
one or both of A and A′ is

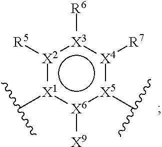

X², X⁴ and X⁶ are carbon; and
one of X¹, X³ and X⁵ is nitrogen and two of X¹, X³ and X⁵ are carbon, or
two of X¹, X³ and X⁵ are nitrogen and one of X¹, X³ and X⁵ is carbon, or
X¹, X³ and X⁵ are nitrogen.

In some embodiments, where the compound is of Formula I or II,
one or both of A and A′ is

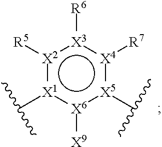

five of X¹, X², X³, X⁴, X⁵ and X⁶ are carbon; and
one of X¹, X², X³, X⁴, X⁵ and X⁶ is nitrogen.

In some embodiments, where the compound is of Formula I or II,
one or both of A and A′ is

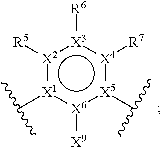

X¹, X², X⁴, X⁵ and X⁶ are carbon;
R⁵, R⁷ and R⁹ are hydrogen; and
R⁶ is methyl.

In some embodiments, where the compound is of Formula I or II,
one or both of A and A′ is

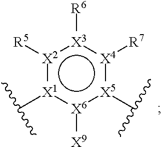

X¹, X², X⁴, X⁵ and X⁶ are carbon;
X³ is nitrogen;
R⁵, R⁷ and R⁹ are hydrogen; and
R⁶ is methyl.

In some embodiments, where the compound is of Formula I or II,
one or both of A and A′ is

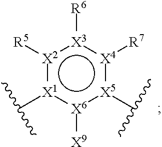

X¹, X², X⁴, X⁵ and X⁶ are carbon;
X³ is nitrogen; and
R⁵, R⁶, R⁷ and R⁹ are hydrogen.

In some embodiments, where the compound is of Formula I or II,
one or both of A and A′ is

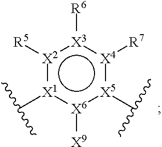

and
X¹ and X⁵ are carbon.

In some embodiments, where the compound is of Formula I or II,
one or both of A and A′ is

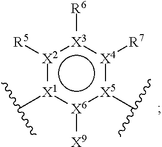

and
X¹, X², X³, X⁴, X⁵ and X⁶ are carbon. In some embodiments, R⁵, R⁷ and R⁹ are hydrogen, and R⁶ is methyl.

In some embodiments, where the compound is of Formula I or II,
one or both of A and A′ is

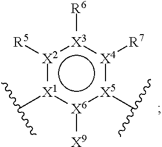

and
X¹ or X⁵, or X¹ and X⁵, are nitrogen.

In some embodiments, where the compound is of Formula I or II,
one or both of A and A′ is

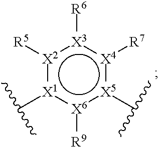

and
one, two or three of X¹, X³ and X⁵, are nitrogen.

In some embodiments, where the compound is of Formula I or II, one or more of R⁵, R⁶, R⁷, R⁸, and R⁹ are halogen. In some embodiments, the halogen is F or Cl. In some embodiments, wherein Z and Z′ are O. In some embodiments, each of R¹ and R³ independently is hydrogen, CH₃ or C₂H₅.

In some embodiments, where Y is M, and/or Y is M¹, and optionally R_{L} is —CH₂—CH₂—, each of R¹ and R² independently is hydrogen, C1-C2 alkyl, or C1-C2 alkyl substituted with one or more halogen atoms. In some embodiments, both R¹ and R² are CH₃ or C₂H₅. In some embodiments, both R¹ and R² are CH₃. In some embodiments, R¹ and R² are H.

In some embodiments, where the compound is of Formula I or II, A and A′ are phenyl and Y is M or M¹. In some embodiments, each of R¹ and R² independently is hydrogen, CH₃ or C₂H₅.

In some embodiments, where Y is M, and/or Y is M¹, and optionally R_{L} is —CH₂—CH₂—,
Z and Z′ are O;
Y is M;
R¹ and R² are CH₃; and
A and A′ are

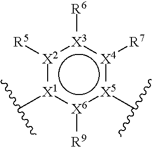

and are the same. In some embodiments, X¹, X², X⁴, X⁵ and X⁶ are carbon; X³ is nitrogen; R⁵, R⁷ and R⁹ are hydrogen; and R⁶ is CH₃.

In some embodiments, the compound is of Formula I or II, wherein A and A′ are phenyl, Y is M, and R¹ and R² are CH₃.

In some embodiments, the compound is of Formula I or II, wherein A and A′ are phenyl, Y is M, Z and Z′ are O, and R¹ and R² are C₂H₅.

In some embodiments, the compound is of Formula I or II, wherein A and A′ are pyridinyl, Y is M, Z and Z′ are O, and R¹ and R² are CH₃.

In some embodiments, where the compound is of Formula I or II, and optionally Y is Q or Q1, each of R¹, R², R³, and R⁴ independently is hydrogen, C1-C2 alkyl, or C1-C2 alkyl substituted with one or more halogen atoms. In some embodiments, each of R¹, R², R³, and R⁴ independently is hydrogen, CH₃ or C₂H₅. In some embodiments, R¹, R², R³, and R⁴ are H. In some embodiments, one of R¹, R², R³ and R⁴ is nonexistent. In some embodiments, R¹ is nonexistent and R2, R³ and R⁴ are H, or R³ is nonexistent and R1, R² and R⁴ are H.

Provided herein in some embodiments is a compound of Formula II having the following structural formula

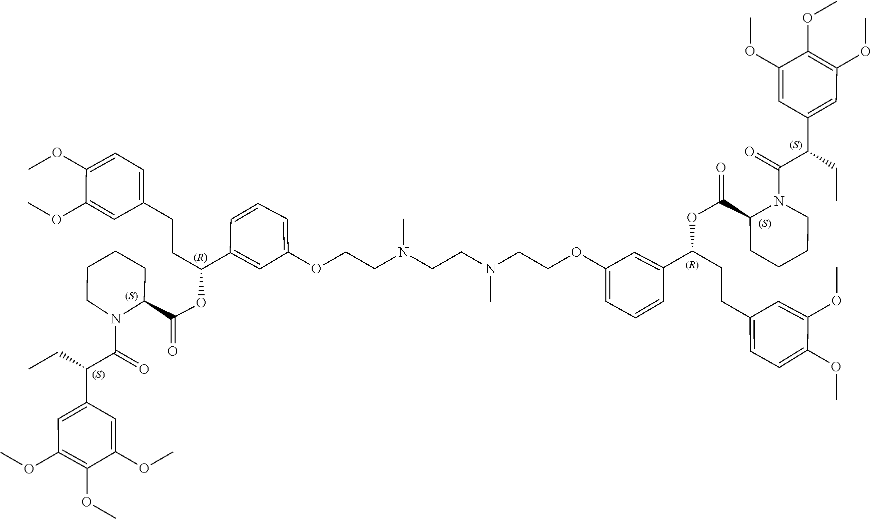

In some embodiments, provided herein are compounds of Formula I or II, wherein Y is L. In some embodiments, Y is

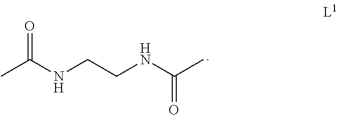

In some embodiments, where Y is L or L¹, A and A′ are

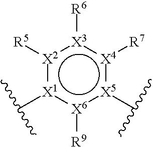

and are the same. In some embodiments, one, two or three of X¹, X², X³, X⁴, X⁵ and X⁶ are nitrogen. In some embodiments, five of X¹, X², X³, X⁴, X⁵ and X⁶ are carbon and one of X¹, X², X³, X⁴, X⁵ and X⁶ is nitrogen. In some embodiments, X¹, X², X⁴, X⁵ and X⁶ are carbon and X³ is nitrogen. In some embodiments, X¹ and X⁵ are carbon. In some embodiments, A and A′ are substituted with a halogen. In some embodiments, the halogen is F or Cl. In some embodiments, Z and Z′ are O. In some embodiments, where X¹, X², X⁴, X⁵ and X⁶ are carbon and X³ is nitrogen, R⁶ is CH₃ or C₂H₅. In some embodiments, where X¹, X², X⁴, X⁵ and X⁶ are carbon and X³ is nitrogen, R⁶ is CH₃.

In some embodiments, where the compound is of Formula I or II, R⁵, R⁶, R⁷ or R⁸ is not present or is hydrogen or methyl.

The following tables provide non-limiting examples of some embodiments of the present compounds, where the compound is of Formula I or II:

TABLE Y = M

| Embodiment | Z | Y | 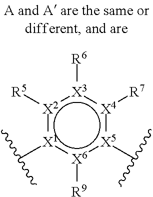 | R⁵, R⁶, R⁷, R⁹ | R^{L} | R¹ and R² are the same or different and are |
|---|---|---|---|---|---|---|
| 1 | O | M | X¹, X², X³, X⁴, X⁵ and X⁶ are | R⁵, R⁶, R⁷, and | lower alkylene, | hydrogen, C1-C2 alkyl, or C1-C2 alkyl |
| | | | carbon | R⁹ are H | alkenylene, alkynylene, | substituted with one or more halogen |
| | | | | | or acyl or aryl, which is | atoms |
| | | | | | optionally substituted | |
| | | | | | with hydroxyl, alkoxyl, | |
| | | | | | amino, alkylamino, thiol, | |
| | | | | | thioalkyl, or halogen | |
| 2 | O | M | X¹, X², X³, X⁴, X⁵ and X⁶ are | R⁵, R⁶, R⁷, and | lower alkylene, | CH₃ or C₂H₅ |
| | | | carbon | R⁹ are H | alkenylene, alkynylene, | |
| | | | | | or acyl or aryl, which is | |
| | | | | | optionally substituted | |
| | | | | | with hydroxyl, alkoxyl, | |
| | | | | | amino, alkylamino, thiol, | |
| | | | | | thioalkyl, or halogen | |
| 3 | O | M | X¹, X², X³, X⁴, X⁵ and X⁶ are | R⁵, R⁶, R⁷, and | lower alkylene, | CH₃ |
| | | | carbon | R⁹ are H | alkenylene, alkynylene, | |
| | | | | | or acyl or aryl, which is | |
| | | | | | optionally substituted | |
| | | | | | with hydroxyl, alkoxyl, | |
| | | | | | amino, alkylamino, thiol, | |
| | | | | | thioalkyl, or halogen | |
| 4 | O | M | X¹, X², X³, X⁴, X⁵ and X⁶ are | R⁵, R⁶, R⁷, and | —CH₂—CH₂— | hydrogen, C1-C2 alkyl, or C1-C2 alkyl |
| | | | carbon | R⁹ are H | | substituted with one or more halogen |
| | | | | | | atoms |
| 5 | O | M | X¹, X², X³, X⁴, X⁵ and X⁶ are | R⁵, R⁶, R⁷, and | —CH₂—CH₂— | CH₃ or C₂H₅ |
| | | | carbon | R⁹ are H | | |
| 6 | O | M | X¹, X², X³, X⁴, X⁵ and X⁶ are | R⁵, R⁶, R⁷, and | —CH₂—CH₂— | CH₃ |
| | | | carbon | R⁹ are H | | |
| 7 | O | M | X¹, X², X³, X⁴, X⁵ and X⁶ are | R⁵, R⁷, and R⁹ | lower alkylene, | hydrogen, C1-C2 alkyl, or C1-C2 alkyl |
| | | | carbon | are H, R⁶ is | alkenylene, alkynylene, | substituted with one or more halogen |
| | | | | methyl | or acyl or aryl, which is | atoms |
| | | | | | optionally substituted | |
| | | | | | with hydroxyl, alkoxyl, | |
| | | | | | amino, alkylamino, thiol, | |
| | | | | | thioalkyl, or halogen | |
| 8 | O | M | X¹, X², X³, X⁴, X⁵ and X⁶ are | R⁵, R⁷, and R⁹ | lower alkylene, | CH₃ or C₂H₅ |
| | | | carbon | are H, R⁶ is | alkenylene, alkynylene, | |
| | | | | methyl | or acyl or aryl, which is | |
| | | | | | optionally substituted | |
| | | | | | with hydroxyl, alkoxyl, | |
| | | | | | amino, alkylamino, thiol, | |
| | | | | | thioalkyl, or halogen | |
| 9 | O | M | X¹, X², X³, X⁴, X⁵ and X⁶ are | R⁵, R⁷, and R⁹ | lower alkylene, | CH₃ |
| | | | carbon | are H, R⁶ is | alkenylene, alkynylene, | |
| | | | | methyl | or acyl or aryl, which is | |
| | | | | | optionally substituted | |
| | | | | | with hydroxyl, alkoxyl, | |
| | | | | | amino, alkylamino, thiol, | |
| | | | | | thioalkyl, or halogen | |
| 10 | O | M | X¹, X², X³, X⁴, X⁵ and X⁶ are | R⁵, R⁷, and R⁹ | —CH₂—CH₂— | hydrogen, C1-C2 alkyl, or C1-C2 alkyl |
| | | | carbon | are H, R⁶ is | | substituted with one or more halogen |
| | | | | methyl | | atoms |
| 11 | O | M | X¹, X², X³, X⁴, X⁵ and X⁶ are | R⁵, R⁷, and R⁹ | —CH₂—CH₂— | CH₃ or C₂H₅ |
| | | | carbon | are H, R⁶ is | | |
| | | | | methyl | | |
| 12 | O | M | X¹, X², X³, X⁴, X⁵ and X⁶ are | R⁵, R⁷, and R⁹ | —CH₂—CH₂— | CH₃ |
| | | | carbon | are H, R⁶ is | | |
| | | | | methyl | | |
| 13 | O | M | X¹, X², X⁴, X⁵ and X⁶ are | R⁵, R⁶, R⁷, and | lower alkylene, | hydrogen, C1-C2 alkyl, or C1-C2 alkyl |
| | | | carbon; | R⁹ are H | alkenylene, alkynylene, | substituted with one or more halogen |
| | | | X³ is nitrogen; and | | or acyl or aryl, which is | atoms |
| | | | R⁵, R⁶, R⁷ and R⁹ are | | optionally substituted | |
| | | | hydrogen | | with hydroxyl, alkoxyl, | |
| | | | | | amino, alkylamino, thiol, | |
| | | | | | thioalkyl, or halogen | |
| 14 | O | M | X¹, X², X⁴, X⁵ and X⁶ are | R⁵, R⁶, R⁷, and | lower alkylene, | CH₃ or C₂H₅ |
| | | | carbon; | R⁹ are H | alkenylene, alkynylene, | |
| | | | X³ is nitrogen; and | | or acyl or aryl, which is | |
| | | | R⁵, R⁶, R⁷ and R⁹ are | | optionally substituted | |
| | | | hydrogen | | with hydroxyl, alkoxyl, | |
| | | | | | amino, alkylamino, thiol, | |
| | | | | | thioalkyl, or halogen | |
| 15 | O | M | X¹, X², X⁴, X⁵ and X⁶ are | R⁵, R⁶, R⁷, and | lower alkylene, | CH₃ |
| | | | carbon; | R⁹ are H | alkenylene, alkynylene, | |
| | | | X³ is nitrogen; and | | or acyl or aryl, which is | |
| | | | R⁵, R⁶, R⁷ and R⁹ are | | optionally substituted | |
| | | | hydrogen | | with hydroxyl, alkoxyl, | |
| | | | | | amino, alkylamino, thiol, | |
| | | | | | thioalkyl, or halogen | |
| 16 | O | M | X¹, X², X⁴, X⁵ and X⁶ are | R⁵, R⁶, R⁷, and | —CH₂—CH₂— | hydrogen, C1-C2 alkyl, or C1-C2 alkyl |
| | | | carbon; | R⁹ are H | | substituted with one or more halogen |
| | | | X³ is nitrogen; and | | | atoms |
| | | | R⁵, R⁶, R⁷ and R⁹ are | | | |
| | | | hydrogen | | | |
| 17 | O | M | X¹, X², X⁴, X⁵ and X⁶ are | R⁵, R⁶, R⁷, and | —CH₂—CH₂— | CH₃ or C₂H₅ |
| | | | carbon; | R⁹ are H | | |
| | | | X³ is nitrogen; and | | | |
| | | | R⁵, R⁶, R⁷ and R⁹ are | | | |
| | | | hydrogen | | | |
| 18 | O | M | X¹, X², X⁴, X⁵ and X⁶ are | R⁵, R⁶, R⁷, and | —CH₂—CH₂— | CH₃ |
| | | | carbon; | R⁹ are H | | |
| | | | X³ is nitrogen; and | | | |
| | | | R⁵, R⁶, R⁷ and R⁹ are | | | |
| | | | hydrogen | | | |
| 19 | O | M | X¹, X², X⁴, X⁵ and X⁶ are | R⁵, R⁷, and R⁹ | lower alkylene, | hydrogen, C1-C2 alkyl, or C1-C2 alkyl |
| | | | carbon; | are H, R⁶ is | alkenylene, alkynylene, | substituted with one or more halogen |
| | | | X³ is nitrogen; and | methyl | or acyl or aryl, which is | atoms |
| | | | R⁵, R⁶, R⁷ and R⁹ are | | optionally substituted | |
| | | | hydrogen | | with hydroxyl, alkoxyl, | |
| | | | | | amino, alkylamino, thiol, | |
| | | | | | thioalkyl, or halogen | |
| 20 | O | M | X¹, X², X⁴, X⁵ and X⁶ are | R⁵, R⁷, and R⁹ | lower alkylene, | CH₃ or C₂H₅ |
| | | | carbon; | are H, R⁶ is | alkenylene, alkynylene, | |
| | | | X³ is nitrogen; and | methyl | or acyl or aryl, which is | |
| | | | R⁵, R⁶, R⁷ and R⁹ are | | optionally substituted | |
| | | | hydrogen | | with hydroxyl, alkoxyl, | |
| | | | | | amino, alkylamino, thiol, | |
| | | | | | thioalkyl, or halogen | |
| 21 | O | M | X¹, X², X⁴, X⁵ and X⁶ are | R⁵, R⁷, and R⁹ | lower alkylene, | CH₃ |
| | | | carbon; | are H, R⁶ is | alkenylene, alkynylene, | |
| | | | X³ is nitrogen; and | methyl | or acyl or aryl, which is | |
| | | | R⁵, R⁶, R⁷ and R⁹ are | | optionally substituted | |
| | | | hydrogen | | with hydroxyl, alkoxyl, | |
| | | | | | amino, alkylamino, thiol, | |
| | | | | | thioalkyl, or halogen | |
| 22 | O | M | X¹, X², X⁴, X⁵ and X⁶ are | R⁵, R⁷, and R⁹ | —CH₂—CH₂— | hydrogen, C1-C2 alkyl, or C1-C2 alkyl |
| | | | carbon; | are H, R⁶ is | | substituted with one or more halogen |
| | | | X³ is nitrogen; and | methyl | | atoms |
| | | | R⁵, R⁶, R⁷ and R⁹ are | | | |
| | | | hydrogen | | | |
| 23 | O | M | X¹, X², X⁴, X⁵ and X⁶ are | R⁵, R⁷, and R⁹ | —CH₂—CH₂— | CH₃ or C₂H₅ |
| | | | carbon; | are H, R⁶ is | | |
| | | | X³ is nitrogen; and | methyl | | |
| | | | R⁵, R⁶, R⁷ and R⁹ are | | | |
| | | | hydrogen | | | |
| 24 | O | M | X¹, X², X⁴, X⁵ and X⁶ are | R⁵, R⁷, and R⁹ | —CH₂—CH₂— | CH₃ |
| | | | carbon; | are H, R⁶ is | | |
| | | | X³ is nitrogen; and | methyl | | |
| | | | R⁵, R⁶, R⁷ and R⁹ are | | | |
| | | | hydrogen | | | |
| 25 | O | M | one, two or three of X¹, X³ and | R⁵, R⁶, R⁷, and | lower alkylene, | hydrogen, C1-C2 alkyl, or C1-C2 alkyl |
| | | | X⁵, are nitrogen, and two, one | R⁹ are H | alkenylene, alkynylene, | substituted with one or more halogen |
| | | | or none of X¹, X³ and X⁵, are | | or acyl or aryl, which is | atoms |
| | | | carbon | | optionally substituted | |
| | | | | | with hydroxyl, alkoxyl, | |
| | | | | | amino, alkylamino, thiol, | |
| | | | | | thioalkyl, or halogen | |
| 26 | O | M | one, two or three of X¹, X³ and | R⁵, R⁶, R⁷, and | lower alkylene, | CH₃ or C₂H₅ |
| | | | X⁵, are nitrogen, and two, one | R⁹ are H | alkenylene, alkynylene, | |
| | | | or none of X¹, X³ and X⁵, are | | or acyl or aryl, which is | |
| | | | carbon | | optionally substituted | |
| | | | | | with hydroxyl, alkoxyl, | |
| | | | | | amino, alkylamino, thiol, | |
| | | | | | thioalkyl, or halogen | |
| 27 | O | M | one, two or three of X¹, X³ and | R⁵, R⁶, R⁷, and | lower alkylene, | CH₃ |
| | | | X⁵, are nitrogen, and two, one | R⁹ are H | alkenylene, alkynylene, | |
| | | | or none of X¹, X³ and X⁵, are | | or acyl or aryl, which is | |
| | | | carbon | | optionally substituted | |
| | | | | | with hydroxyl, alkoxyl, | |
| | | | | | amino, alkylamino, thiol, | |
| | | | | | thioalkyl, or halogen | |
| 28 | O | M | one, two or three of X¹, X³ and | R⁵, R⁶, R⁷, and | —CH₂—CH₂— | hydrogen, C1-C2 alkyl, or C1-C2 alkyl |
| | | | X⁵, are nitrogen, and two, one | R⁹ are H | | substituted with one or more halogen |
| | | | or none of X¹, X³ and X⁵, are | | | atoms |
| | | | carbon | | | |
| 29 | O | M | one, two or three of X¹, X³ and | R⁵, R⁶, R⁷, and | —CH₂—CH₂— | CH₃ or C₂H₅ |
| | | | X⁵, are nitrogen, and two, one | R⁹ are H | | |
| | | | or none of X¹, X³ and X⁵, are | | | |
| | | | carbon | | | |
| 30 | O | M | one, two or three of X¹, X³ and | R⁵, R⁶, R⁷, and | —CH₂—CH₂— | CH₃ |
| | | | X⁵, are nitrogen, and two, one | R⁹ are H | | |
| | | | or none of X¹, X³ and X⁵, are | | | |
| | | | carbon | | | |
| 31 | O | M | one, two or three of X¹, X³ and | R⁵, R⁷, and R⁹ | lower alkylene, | hydrogen, C1-C2 alkyl, or C1-C2 alkyl |
| | | | X⁵, are nitrogen, and two, one | are H, R⁶ is | alkenylene, alkynylene, | substituted with one or more halogen |
| | | | or none of X¹, X³ and X⁵, are | methyl | or acyl or aryl, which is | atoms |
| | | | carbon | | optionally substituted | |
| | | | | | with hydroxyl, alkoxyl, | |
| | | | | | amino, alkylamino, thiol, | |
| | | | | | thioalkyl, or halogen | |
| 32 | O | M | one, two or three of X¹, X³ and | R⁵, R⁷, and R⁹ | lower alkylene, | CH₃ or C₂H₅ |
| | | | X⁵, are nitrogen, and two, one | are H, R⁶ is | alkenylene, alkynylene, | |
| | | | or none of X¹, X³ and X⁵, are | methyl | or acyl or aryl, which is | |
| | | | carbon | | optionally substituted | |
| | | | | | with hydroxyl, alkoxyl, | |
| | | | | | amino, alkylamino, thiol, | |
| | | | | | thioalkyl, or halogen | |
| 33 | O | M | one, two or three of X¹, X³ and | R⁵, R⁷, and R⁹ | lower alkylene, | CH₃ |
| | | | X⁵, are nitrogen, and two, one | are H, R⁶ is | alkenylene, alkynylene, | |
| | | | or none of X¹, X³ and X⁵, are | methyl | or acyl or aryl, which is | |
| | | | carbon | | optionally substituted | |
| | | | | | with hydroxyl, alkoxyl, | |
| | | | | | amino, alkylamino, thiol, | |
| | | | | | thioalkyl, or halogen | |
| 34 | O | M | one, two or three of X¹, X³ and | R⁵, R⁷, and R⁹ | —CH₂—CH₂— | hydrogen, C1-C2 alkyl, or C1-C2 alkyl |
| | | | X⁵, are nitrogen, and two, one | are H, R⁶ is | | substituted with one or more halogen |
| | | | or none of X¹, X³ and X⁵, are | methyl | | atoms |
| | | | carbon | | | |
| 35 | O | M | one, two or three of X¹, X³ and | R⁵, R⁷, and R⁹ | —CH₂—CH₂— | CH₃ or C₂H₅ |
| | | | X⁵, are nitrogen, and two, one | are H, R⁶ is | | |
| | | | or none of X¹, X³ and X⁵, are | methyl | | |
| | | | carbon | | | |
| 36 | O | M | one, two or three of X¹, X³ and | R⁵, R⁷, and R⁹ | —CH₂—CH₂— | CH₃ |
| | | | X⁵, are nitrogen, and two, one | are H, R⁶ is | | |
| | | | or none of X¹, X³ and X⁵, are | methyl | | |
| | | | carbon | | | |

TABLE Y = L

| Embodiment | Z | Y | 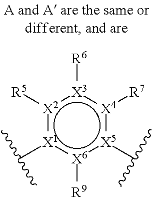 | R⁵, R⁶, R⁷, R⁹ | R^{L} |
|---|---|---|---|---|---|
| 101 | O | L | X¹, X², X³, X⁴, X⁵ and X⁶ are | R⁵, R⁶, R⁷, and | lower alkylene, |
| | | | carbon | R⁹ are H | alkenylene, alkynylene, |
| | | | | | or acyl or aryl, which is |
| | | | | | optionally substituted |
| | | | | | with hydroxyl, alkoxyl, |
| | | | | | amino, alkylamino, thiol, |
| | | | | | thioalkyl, or halogen, |
| | | | | | with the caveat that R^{L} is |
| | | | | | not |
| | | | | | —CH₂—CH₂— |
| 102 | O | L | X¹, X², X³, X⁴, X⁵ and X⁶ are | R⁵, R⁷, and R⁹ | lower alkylene, |
| | | | carbon | are H, R⁶ is | alkenylene, alkynylene, |
| | | | | methyl | or acyl or aryl, which is |
| | | | | | optionally substituted |
| | | | | | with hydroxyl, alkoxyl, |
| | | | | | amino, alkylamino, thiol, |
| | | | | | thioalkyl, or halogen |
| 103 | O | L | X¹, X², X³, X⁴, X⁵ and X⁶ are | R⁵, R⁷, and R⁹ | —CH₂—CH₂— |
| | | | carbon | are H, R⁶ is | |
| | | | | methyl | |
| 104 | O | L | X¹, X², X⁴, X⁵ and X⁶ are | R⁵, R⁶, R⁷, and | lower alkylene, |
| | | | carbon; | R⁹ are H | alkenylene, alkynylene, |
| | | | X³ is nitrogen; and | | or acyl or aryl, which is |
| | | | R⁵, R⁶, R⁷ and R⁹ are | | optionally substituted |
| | | | hydrogen | | with hydroxyl, alkoxyl, |
| | | | | | amino, alkylamino, thiol, |
| | | | | | thioalkyl, or halogen |
| 105 | O | L | X¹, X², X⁴, X⁵ and X⁶ are | R⁵, R⁶, R⁷, and | —CH₂—CH₂— |
| | | | carbon; | R⁹ are H | |
| | | | X³ is nitrogen; and | | |
| | | | R⁵, R⁶, R⁷ and R⁹ are | | |
| | | | hydrogen | | |
| 106 | O | L | X¹, X², X⁴, X⁵ and X⁶ are | R⁵, R⁷, and R⁹ | lower alkylene, |
| | | | carbon; | are H, R⁶ is | alkenylene, alkynylene, |
| | | | X³ is nitrogen; and | methyl | or acyl or aryl, which is |
| | | | R⁵, R⁶, R⁷ and R⁹ are | | optionally substituted |
| | | | hydrogen | | with hydroxyl, alkoxyl, |
| | | | | | amino, alkylamino, thiol, |
| | | | | | thioalkyl, or halogen |
| 107 | O | L | X¹, X², X⁴, X⁵ and X⁶ are | R⁵, R⁷, and R⁹ | —CH₂—CH₂— |
| | | | carbon; | are H, R⁶ is | |
| | | | X³ is nitrogen; and | methyl | |
| | | | R⁵, R⁶, R⁷ and R⁹ are | | |
| | | | hydrogen | | |
| 108 | O | L | one, two or three of X¹, X³ and | R⁵, R⁶, R⁷, and | lower alkylene, |
| | | | X⁵, are nitrogen, and two, one | R⁹ are H | alkenylene, alkynylene, |
| | | | or none of X¹, X³ and X⁵, are | | or acyl or aryl, which is |
| | | | carbon | | optionally substituted |
| | | | | | with hydroxyl, alkoxyl, |
| | | | | | amino, alkylamino, thiol, |
| | | | | | thioalkyl, or halogen |
| 109 | O | L | one, two or three of X¹, X³ and | R⁵, R⁶, R⁷, and | —CH₂—CH₂— |
| | | | X⁵, are nitrogen, and two, one | R⁹ are H | |
| | | | or none of X¹, X³ and X⁵, are | | |
| | | | carbon | | |
| 110 | O | L | one, two or three of X¹, X³ and | R⁵, R⁷, and R⁹ | lower alkylene, |
| | | | X⁵, are nitrogen, and two, one | are H, R⁶ is | alkenylene, alkynylene, |
| | | | or none of X¹, X³ and X⁵, are | methyl | or acyl or aryl, which is |
| | | | carbon | | optionally substituted |
| | | | | | with hydroxyl, alkoxyl, |
| | | | | | amino, alkylamino, thiol, |
| | | | | | thioalkyl, or halogen |
| 111 | O | L | one, two or three of X¹, X³ and | R⁵, R⁷, and R⁹ | —CH₂—CH₂— |
| | | | X⁵, are nitrogen, and two, one | are H, R⁶ is | |
| | | | or none of X¹, X³ and X⁵, are | methyl | |
| | | | carbon | | |

TABLE Y = Q

| Embodiment | Z | Y | 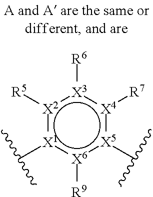 | R⁵, R⁶, R⁷, R⁹ | R^{L} | R¹ and R³ are the same or different and are | R² and R⁴ are the same or different and are |
|---|---|---|---|---|---|---|---|
| 201 | O | Q | X¹, X², X³, X⁴, | R⁵, R⁶, R⁷, and | lower alkylene, alkenylene, | hydrogen, C1-C2 alkyl, or | hydrogen, C1-C2 |
| | | | X⁵ and X⁶ are | R⁹ are H | alkynylene, or acyl or aryl, | C1-C2 alkyl substituted | alkyl, or C1-C2 alkyl |
| | | | carbon | | which is optionally substituted | with one or more | substituted with one or |
| | | | | | with hydroxyl, alkoxyl, amino, | halogen atoms | more halogen atoms |
| | | | | | alkylamino, thiol, thioalkyl, or | | |
| | | | | | halogen | | |
| 202 | O | Q | X¹, X², X³, X⁴, | R⁵, R⁶, R⁷, and | lower alkylene, alkenylene, | hydrogen, C1-C2 alkyl, or | CH₃ or C₂H₅ |
| | | | X⁵ and X⁶ are | R⁹ are H | alkynylene, or acyl or aryl, | C1-C2 alkyl substituted | |
| | | | carbon | | which is optionally substituted | with one or more | |
| | | | | | with hydroxyl, alkoxyl, amino, | halogen atoms | |
| | | | | | alkylamino, thiol, thioalkyl, or | | |
| | | | | | halogen | | |
| 203 | O | Q | X¹, X², X³, X⁴, | R⁵, R⁶, R⁷, and | lower alkylene, alkenylene, | hydrogen, C1-C2 alkyl, or | H |
| | | | X⁵ and X⁶ are | R⁹ are H | alkynylene, or acyl or aryl, | C1-C2 alkyl substituted | |
| | | | carbon | | which is optionally substituted | with one or more | |
| | | | | | with hydroxyl, alkoxyl, amino, | halogen atoms | |
| | | | | | alkylamino, thiol, thioalkyl, or | | |
| | | | | | halogen | | |
| 204 | O | Q | X¹, X², X³, X⁴, | R⁵, R⁶, R⁷, and | lower alkylene, alkenylene, | CH₃ or C₂H₅ | hydrogen, C1-C2 |
| | | | X⁵ and X⁶ are | R⁹ are H | alkynylene, or acyl or aryl, | | alkyl, or C1-C2 alkyl |
| | | | carbon | | which is optionally substituted | | substituted with one or |
| | | | | | with hydroxyl, alkoxyl, amino, | | more halogen atoms |
| | | | | | alkylamino, thiol, thioalkyl, or | | |
| | | | | | halogen | | |
| 205 | O | Q | X¹, X², X³, X⁴, | R⁵, R⁶, R⁷, and | lower alkylene, alkenylene, | CH₃ or C₂H₅ | CH₃ or C₂H₅ |
| | | | X⁵ and X⁶ are | R⁹ are H | alkynylene, or acyl or aryl, | | |
| | | | carbon | | which is optionally substituted | | |
| | | | | | with hydroxyl, alkoxyl, amino, | | |
| | | | | | alkylamino, thiol, thioalkyl, or | | |
| | | | | | halogen | | |
| 206 | O | Q | X¹, X², X³, X⁴, | R⁵, R⁶, R⁷, and | lower alkylene, alkenylene, | CH₃ or C₂H₅ | H |
| | | | X⁵ and X⁶ are | R⁹ are H | alkynylene, or acyl or aryl, | | |
| | | | carbon | | which is optionally substituted | | |
| | | | | | with hydroxyl, alkoxyl, amino, | | |
| | | | | | alkylamino, thiol, thioalkyl, or | | |
| | | | | | halogen | | |
| 207 | O | Q | X¹, X², X³, X⁴, | R⁵, R⁶, R⁷, and | lower alkylene, alkenylene, | CH₃ | hydrogen, C1-C2 |
| | | | X⁵ and X⁶ are | R⁹ are H | alkynylene, or acyl or aryl, | | alkyl, or C1-C2 alkyl |
| | | | carbon | | which is optionally substituted | | substituted with one or |
| | | | | | with hydroxyl, alkoxyl, amino, | | more halogen atoms |
| | | | | | alkylamino, thiol, thioalkyl, or | | |
| | | | | | halogen | | |
| 208 | O | Q | X¹, X², X³, X⁴, | R⁵, R⁶, R⁷, and | lower alkylene, alkenylene, | CH₃ | CH₃ or C₂H₅ |
| | | | X⁵ and X⁶ are | R⁹ are H | alkynylene, or acyl or aryl, | | |
| | | | carbon | | which is optionally substituted | | |
| | | | | | with hydroxyl, alkoxyl, amino, | | |
| | | | | | alkylamino, thiol, thioalkyl, or | | |
| | | | | | halogen | | |
| 209 | O | Q | X¹, X², X³, X⁴, | R⁵, R⁶, R⁷, and | lower alkylene, alkenylene, | CH₃ | H |
| | | | X⁵ and X⁶ are | R⁹ are H | alkynylene, or acyl or aryl, | | |
| | | | carbon | | which is optionally substituted | | |
| | | | | | with hydroxyl, alkoxyl, amino, | | |
| | | | | | alkylamino, thiol, thioalkyl, or | | |
| | | | | | halogen | | |
| 210 | O | Q | X¹, X², X³, X⁴, | R⁵, R⁶, R⁷, and | —CH₂—CH₂— | hydrogen, C1-C2 alkyl, or | hydrogen, C1-C2 |
| | | | X⁵ and X⁶ are | R⁹ are H | | C1-C2 alkyl substituted | alkyl, or C1-C2 alkyl |
| | | | carbon | | | with one or more | substituted with one or |
| | | | | | | halogen atoms | more halogen atoms |
| 211 | O | Q | X¹, X², X³, X⁴, | R⁵, R⁶, R⁷, and | —CH₂—CH₂— | hydrogen, C1-C2 alkyl, or | CH₃ or C₂H₅ |
| | | | X⁵ and X⁶ are | R⁹ are H | | C1-C2 alkyl substituted | |
| | | | carbon | | | with one or more | |
| | | | | | | halogen atoms | |
| 212 | O | Q | X¹, X², X³, X⁴, | R⁵, R⁶, R⁷, and | —CH₂—CH₂— | hydrogen, C1-C2 alkyl, or | H |
| | | | X⁵ and X⁶ are | R⁹ are H | | C1-C2 alkyl substituted | |
| | | | carbon | | | with one or more | |
| | | | | | | halogen atoms | |
| 213 | O | Q | X¹, X², X³, X⁴, | R⁵, R⁶, R⁷, and | —CH₂—CH₂— | CH₃ or C₂H₅ | hydrogen, C1-C2 |
| | | | X⁵ and X⁶ are | R⁹ are H | | | alkyl, or C1-C2 alkyl |
| | | | carbon | | | | substituted with one or |
| | | | | | | | more halogen atoms |
| 214 | O | Q | X¹, X², X³, X⁴, | R⁵, R⁶, R⁷, and | —CH₂—CH₂— | CH₃ or C₂H₅ | CH₃ or C₂H₅ |
| | | | X⁵ and X⁶ are | R⁹ are H | | | |
| | | | carbon | | | | |
| 215 | O | Q | X¹, X², X³, X⁴, | R⁵, R⁶, R⁷, and | —CH₂—CH₂— | CH₃ or C₂H₅ | H |
| | | | X⁵ and X⁶ are | R⁹ are H | | | |
| | | | carbon | | | | |
| 216 | O | Q | X¹, X², X³, X⁴, | R⁵, R⁶, R⁷, and | —CH₂—CH₂— | CH₃ | hydrogen, C1-C2 |
| | | | X⁵ and X⁶ are | R⁹ are H | | | alkyl, or C1-C2 alkyl |
| | | | carbon | | | | substituted with one or |
| | | | | | | | more halogen atoms |
| 217 | O | Q | X¹, X², X³, X⁴, | R⁵, R⁶, R⁷, and | —CH₂—CH₂— | CH₃ | CH₃ or C₂H₅ |
| | | | X⁵ and X⁶ are | R⁹ are H | | | |
| | | | carbon | | | | |
| 218 | O | Q | X¹, X², X³, X⁴, | R⁵, R⁶, R⁷, and | —CH₂—CH₂— | CH₃ | H |
| | | | X⁵ and X⁶ are | R⁹ are H | | | |
| | | | carbon | | | | |
| 219 | O | Q | X¹, X², X³, X⁴, | R⁵, R⁷, and | lower alkylene, alkenylene, | hydrogen, C1-C2 alkyl, or | hydrogen, C1-C2 |
| | | | X⁵ and X⁶ are | R⁹ are H, R⁶ is | alkynylene, or acyl or aryl, | C1-C2 alkyl substituted | alkyl, or C1-C2 alkyl |
| | | | carbon | methyl | which is optionally substituted | with one or more | substituted with one or |
| | | | | | with hydroxyl, alkoxyl, amino, | halogen atoms | more halogen atoms |
| | | | | | alkylamino, thiol, thioalkyl, or | | |
| | | | | | halogen | | |
| 220 | O | Q | X¹, X², X³, X⁴, | R⁵, R⁷, and | lower alkylene, alkenylene, | hydrogen, C1-C2 alkyl, or | CH₃ or C₂H₅ |
| | | | X⁵ and X⁶ are | R⁹ are H, R⁶ is | alkynylene, or acyl or aryl, | C1-C2 alkyl substituted | |
| | | | carbon | methyl | which is optionally substituted | with one or more | |
| | | | | | with hydroxyl, alkoxyl, amino, | halogen atoms | |
| | | | | | alkylamino, thiol, thioalkyl, or | | |
| | | | | | halogen | | |
| 221 | O | Q | X¹, X², X³, X⁴, | R⁵, R⁷, and | lower alkylene, alkenylene, | hydrogen, C1-C2 alkyl, or | H |
| | | | X⁵ and X⁶ are | R⁹ are H, R⁶ is | alkynylene, or acyl or aryl, | C1-C2 alkyl substituted | |
| | | | carbon | methyl | which is optionally substituted | with one or more | |
| | | | | | with hydroxyl, alkoxyl, amino, | halogen atoms | |
| | | | | | alkylamino, thiol, thioalkyl, or | | |
| | | | | | halogen | | |
| 222 | O | Q | X¹, X², X³, X⁴, | R⁵, R⁷, and | lower alkylene, alkenylene, | CH₃ or C₂H₅ | hydrogen, C1-C2 |
| | | | X⁵ and X⁶ are | R⁹ are H, R⁶ is | alkynylene, or acyl or aryl, | | alkyl, or C1-C2 alkyl |
| | | | carbon | methyl | which is optionally substituted | | substituted with one or |
| | | | | | with hydroxyl, alkoxyl, amino, | | more halogen atoms |
| | | | | | alkylamino, thiol, thioalkyl, or | | |
| | | | | | halogen | | |
| 223 | O | Q | X¹, X², X³, X⁴, | R⁵, R⁷, and | lower alkylene, alkenylene, | CH₃ or C₂H₅ | CH₃ or C₂H₅ |
| | | | X⁵ and X⁶ are | R⁹ are H, R⁶ is | alkynylene, or acyl or aryl, | | |
| | | | carbon | methyl | which is optionally substituted | | |
| | | | | | with hydroxyl, alkoxyl, amino, | | |
| | | | | | alkylamino, thiol, thioalkyl, or | | |
| | | | | | halogen | | |
| 224 | O | Q | X¹, X², X³, X⁴, | R⁵, R⁷, and | lower alkylene, alkenylene, | CH₃ or C₂H₅ | H |
| | | | X⁵ and X⁶ are | R⁹ are H, R⁶ is | alkynylene, or acyl or aryl, | | |
| | | | carbon | methyl | which is optionally substituted | | |
| | | | | | with hydroxyl, alkoxyl, amino, | | |
| | | | | | alkylamino, thiol, thioalkyl, or | | |
| | | | | | halogen | | |
| 225 | O | Q | X¹, X², X³, X⁴, | R⁵, R⁷, and | lower alkylene, alkenylene, | CH₃ | hydrogen, C1-C2 |
| | | | X⁵ and X⁶ are | R⁹ are H, R⁶ is | alkynylene, or acyl or aryl, | | alkyl, or C1-C2 alkyl |
| | | | carbon | methyl | which is optionally substituted | | substituted with one or |
| | | | | | with hydroxyl, alkoxyl, amino, | | more halogen atoms |
| | | | | | alkylamino, thiol, thioalkyl, or | | |
| | | | | | halogen | | |
| 226 | O | Q | X¹, X², X³, X⁴, | R⁵, R⁷, and | lower alkylene, alkenylene, | CH₃ | CH₃ or C₂H₅ |
| | | | X⁵ and X⁶ are | R⁹ are H, R⁶ is | alkynylene, or acyl or aryl, | | |
| | | | carbon | methyl | which is optionally substituted | | |
| | | | | | with hydroxyl, alkoxyl, amino, | | |
| | | | | | alkylamino, thiol, thioalkyl, or | | |
| | | | | | halogen | | |
| 227 | O | Q | X¹, X², X³, X⁴, | R⁵, R⁷, and | lower alkylene, alkenylene, | CH₃ | H |
| | | | X⁵ and X⁶ are | R⁹ are H, R⁶ is | alkynylene, or acyl or aryl, | | |
| | | | carbon | methyl | which is optionally substituted | | |
| | | | | | with hydroxyl, alkoxyl, amino, | | |
| | | | | | alkylamino, thiol, thioalkyl, or | | |
| | | | | | halogen | | |
| 228 | O | Q | X¹, X², X³, X⁴, | R⁵, R⁷, and | —CH₂—CH₂— | hydrogen, C1-C2 alkyl, or | hydrogen, C1-C2 |
| | | | X⁵ and X⁶ are | R⁹ are H, R⁶ is | | C1-C2 alkyl substituted | alkyl, or C1-C2 alkyl |
| | | | carbon | methyl | | with one or more | substituted with one or |
| | | | | | | halogen atoms | more halogen atoms |
| 229 | O | Q | X¹, X², X³, X⁴, | R⁵, R⁷, and | —CH₂—CH₂— | hydrogen, C1-C2 alkyl, or | CH₃ or C₂H₅ |
| | | | X⁵ and X⁶ are | R⁹ are H, R⁶ is | | C1-C2 alkyl substituted | |
| | | | carbon | methyl | | with one or more | |
| | | | | | | halogen atoms | |
| 230 | O | Q | X¹, X², X³, X⁴, | R⁵, R⁷, and | —CH₂—CH₂— | hydrogen, C1-C2 alkyl, or | H |
| | | | X⁵ and X⁶ are | R⁹ are H, R⁶ is | | C1-C2 alkyl substituted | |
| | | | carbon | methyl | | with one or more | |
| | | | | | | halogen atoms | |
| 231 | O | Q | X¹, X², X³, X⁴, | R⁵, R⁷, and | —CH₂—CH₂— | CH₃ or C₂H₅ | hydrogen, C1-C2 |
| | | | X⁵ and X⁶ are | R⁹ are H, R⁶ is | | | alkyl, or C1-C2 alkyl |
| | | | carbon | methyl | | | substituted with one or |
| | | | | | | | more halogen atoms |
| 232 | O | Q | X¹, X², X³, X⁴, | R⁵, R⁷, and | —CH₂—CH₂— | CH₃ or C₂H₅ | CH₃ or C₂H₅ |
| | | | X⁵ and X⁶ are | R⁹ are H, R⁶ is | | | |
| | | | carbon | methyl | | | |
| 233 | O | Q | X¹, X², X³, X⁴, | R⁵, R⁷, and | —CH₂—CH₂— | CH₃ or C₂H₅ | H |
| | | | X⁵ and X⁶ are | R⁹ are H, R⁶ is | | | |
| | | | carbon | methyl | | | |
| 234 | O | Q | X¹, X², X³, X⁴, | R⁵, R⁷, and | —CH₂—CH₂— | CH₃ | hydrogen, C1-C2 |
| | | | X⁵ and X⁶ are | R⁹ are H, R⁶ is | | | alkyl, or C1-C2 alkyl |
| | | | carbon | methyl | | | substituted with one or |
| | | | | | | | more halogen atoms |
| 235 | O | Q | X¹, X², X³, X⁴, | R⁵, R⁷, and | —CH₂—CH₂— | CH₃ | CH₃ or C₂H₅ |
| | | | X⁵ and X⁶ are | R⁹ are H, R⁶ is | | | |
| | | | carbon | methyl | | | |
| 236 | O | Q | X¹, X², X³, X⁴, | R⁵, R⁷, and | —CH₂—CH₂— | CH₃ | H |
| | | | X⁵ and X⁶ are | R⁹ are H, R⁶ is | | | |
| | | | carbon | methyl | | | |
| 237 | O | Q | X¹, X², X⁴, X⁵ and | R⁵, R⁶, R⁷, and | lower alkylene, alkenylene, | hydrogen, C1-C2 alkyl, or | hydrogen, C1-C2 |
| | | | X⁶ are carbon; | R⁹ are H | alkynylene, or acyl or aryl, | C1-C2 alkyl substituted | alkyl, or C1-C2 alkyl |
| | | | X³ is nitrogen; and | | which is optionally substituted | with one or more | substituted with one or |
| | | | R⁵, R⁶, R⁷ and R⁹ are | | with hydroxyl, alkoxyl, amino, | halogen atoms | more halogen atoms |
| | | | hydrogen | | alkylamino, thiol, thioalkyl, or | | CH₃ or C₂H₅ |
| | | | | | halogen | | |
| 238 | O | Q | X¹, X², X⁴, X⁵ and | R⁵, R⁶, R⁷, and | lower alkylene, alkenylene, | hydrogen, C1-C2 alkyl, or | CH₃ or C₂H₅ |
| | | | X⁶ are carbon; | R⁹ are H | alkynylene, or acyl or aryl, | C1-C2 alkyl substituted | |
| | | | X³ is nitrogen; and | | which is optionally substituted | with one or more | |
| | | | R⁵, R⁶, R⁷ and R⁹ are | | with hydroxyl, alkoxyl, amino, | halogen atoms | |
| | | | hydrogen | | alkylamino, thiol, thioalkyl, or | | |
| | | | | | halogen | | |
| 239 | O | Q | X¹, X², X⁴, X⁵ and | R⁵, R⁶, R⁷, and | lower alkylene, alkenylene, | hydrogen, C1-C2 alkyl, or | H |
| | | | X⁶ are carbon; | R⁹ are H | alkynylene, or acyl or aryl, | C1-C2 alkyl substituted | |
| | | | X³ is nitrogen; and | | which is optionally substituted | with one or more | |
| | | | R⁵, R⁶, R⁷ and R⁹ are | | with hydroxyl, alkoxyl, amino, | halogen atoms | |
| | | | hydrogen | | alkylamino, thiol, thioalkyl, or | | |
| | | | | | halogen | | |
| 240 | O | Q | X¹, X², X⁴, X⁵ and | R⁵, R⁶, R⁷, and | lower alkylene, alkenylene, | CH₃ or C₂H₅ | hydrogen, C1-C2 |
| | | | X⁶ are carbon; | R⁹ are H | alkynylene, or acyl or aryl, | | alkyl, or C1-C2 alkyl |
| | | | X³ is nitrogen; and | | which is optionally substituted | | substituted with one or |
| | | | R⁵, R⁶, R⁷ and R⁹ are | | with hydroxyl, alkoxyl, amino, | | more halogen atoms |
| | | | hydrogen | | alkylamino, thiol, thioalkyl, or | | |
| | | | | | halogen | | |
| 241 | O | Q | X¹, X², X⁴, X⁵ and | R⁵, R⁶, R⁷, and | lower alkylene, alkenylene, | CH₃ or C₂H₅ | CH₃ or C₂H₅ |
| | | | X⁶ are carbon; | R⁹ are H | alkynylene, or acyl or aryl, | | |
| | | | X³ is nitrogen; and | | which is optionally substituted | | |
| | | | R⁵, R⁶, R⁷ and R⁹ are | | with hydroxyl, alkoxyl, amino, | | |
| | | | hydrogen | | alkylamino, thiol, thioalkyl, or | | |
| | | | | | halogen | | |
| 242 | O | Q | X¹, X², X⁴, X⁵ and | R⁵, R⁶, R⁷, and | lower alkylene, alkenylene, | CH₃ or C₂H₅ | H |
| | | | X⁶ are carbon; | R⁹ are H | alkynylene, or acyl or aryl, | | |
| | | | X³ is nitrogen; and | | which is optionally substituted | | |
| | | | R⁵, R⁶, R⁷ and R⁹ are | | with hydroxyl, alkoxyl, amino, | | |
| | | | hydrogen | | alkylamino, thiol, thioalkyl, or | | |
| | | | | | halogen | | |
| 243 | O | Q | X¹, X², X⁴, X⁵ and | R⁵, R⁶, R⁷, and | lower alkylene, alkenylene, | CH₃ | hydrogen, C1-C2 |
| | | | X⁶ are carbon; | R⁹ are H | alkynylene, or acyl or aryl, | | alkyl, or C1-C2 alkyl |
| | | | X³ is nitrogen; and | | which is optionally substituted | | substituted with one or |
| | | | R⁵, R⁶, R⁷ and R⁹ are | | with hydroxyl, alkoxyl, amino, | | more halogen atoms |
| | | | hydrogen | | alkylamino, thiol, thioalkyl, or | | |
| | | | | | halogen | | |
| 244 | O | Q | X¹, X², X⁴, X⁵ and | R⁵, R⁶, R⁷, and | lower alkylene, alkenylene, | CH₃ | CH₃ or C₂H₅ |
| | | | X⁶ are carbon; | R⁹ are H | alkynylene, or acyl or aryl, | | |
| | | | X³ is nitrogen; and | | which is optionally substituted | | |
| | | | R⁵, R⁶, R⁷ and R⁹ are | | with hydroxyl, alkoxyl, amino, | | |
| | | | hydrogen | | alkylamino, thiol, thioalkyl, or | | |
| | | | | | halogen | | |
| 245 | O | Q | X¹, X², X⁴, X⁵ and | R⁵, R⁶, R⁷, and | lower alkylene, alkenylene, | CH₃ | H |
| | | | X⁶ are carbon; | R⁹ are H | alkynylene, or acyl or aryl, | | |
| | | | X³ is nitrogen; and | | which is optionally substituted | | |
| | | | R⁵, R⁶, R⁷ and R⁹ are | | with hydroxyl, alkoxyl, amino, | | |
| | | | hydrogen | | alkylamino, thiol, thioalkyl, or | | |
| | | | | | halogen | | |
| 246 | O | Q | X¹, X², X⁴, X⁵ and | R⁵, R⁶, R⁷, and | —CH₂—CH₂— | hydrogen, C1-C2 alkyl, or | hydrogen, C1-C2 |
| | | | X⁶ are carbon; | R⁹ are H | | C1-C2 alkyl substituted | alkyl, or C1-C2 alkyl |
| | | | X³ is nitrogen; and | | | with one or more | substituted with one or |
| | | | R⁵, R⁶, R⁷ and R⁹ are | | | halogen atoms | more halogen atoms |
| | | | hydrogen | | | | |
| 247 | O | Q | X¹, X², X⁴, X⁵ and | R⁵, R⁶, R⁷, and | —CH₂—CH₂— | hydrogen, C1-C2 alkyl, or | CH₃ or C₂H₅ |
| | | | X⁶ are carbon; | R⁹ are H | | C1-C2 alkyl substituted | |
| | | | X³ is nitrogen; and | | | with one or more | |
| | | | R⁵, R⁶, R⁷ and R⁹ are | | | halogen atoms | |
| | | | hydrogen | | | | |
| 248 | O | Q | X¹, X², X⁴, X⁵ and | R⁵, R⁶, R⁷, and | —CH₂—CH₂— | hydrogen, C1-C2 alkyl, or | H |
| | | | X⁶ are carbon; | R⁹ are H | | C1-C2 alkyl substituted | |
| | | | X³ is nitrogen; and | | | with one or more | |
| | | | R⁵, R⁶, R⁷ and R⁹ are | | | halogen atoms | |
| | | | hydrogen | | | | |
| 249 | O | Q | X¹, X², X⁴, X⁵ and | R⁵, R⁶, R⁷, and | —CH₂—CH₂— | CH₃ or C₂H₅ | hydrogen, C1-C2 |
| | | | X⁶ are carbon; | R⁹ are H | | | alkyl, or C1-C2 alkyl |
| | | | X³ is nitrogen; and | | | | substituted with one or |
| | | | R⁵, R⁶, R⁷ and R⁹ are | | | | more halogen atoms |
| | | | hydrogen | | | | |
| 250 | O | Q | X¹, X², X⁴, X⁵ and | R⁵, R⁶, R⁷, and | —CH₂—CH₂— | CH₃ or C₂H₅ | CH₃ or C₂H₅ |
| | | | X⁶ are carbon; | R⁹ are H | | | |
| | | | X³ is nitrogen; and | | | | |
| | | | R⁵, R⁶, R⁷ and R⁹ are | | | | |
| | | | hydrogen | | | | |
| 251 | O | Q | X¹, X², X⁴, X⁵ and | R⁵, R⁶, R⁷, and | —CH₂—CH₂— | CH₃ or C₂H₅ | H |
| | | | X⁶ are carbon; | R⁹ are H | | | |
| | | | X³ is nitrogen; and | | | | |
| | | | R⁵, R⁶, R⁷ and R⁹ are | | | | |
| | | | hydrogen | | | | |
| 252 | O | Q | X¹, X², X⁴, X⁵ and | R⁵, R⁶, R⁷, and | —CH₂—CH₂— | CH₃ | hydrogen, C1-C2 |
| | | | X⁶ are carbon; | R⁹ are H | | | alkyl, or C1-C2 alkyl |
| | | | X³ is nitrogen; and | | | | substituted with one or |
| | | | R⁵, R⁶, R⁷ and R⁹ are | | | | more halogen atoms |
| | | | hydrogen | | | | |
| 253 | O | Q | X¹, X², X⁴, X⁵ and | R⁵, R⁶, R⁷, and | —CH₂—CH₂— | CH₃ | CH₃ or C₂H₅ |
| | | | X⁶ are carbon; | R⁹ are H | | | |
| | | | X³ is nitrogen; and | | | | |
| | | | R⁵, R⁶, R⁷ and R⁹ are | | | | |
| | | | hydrogen | | | | |
| 254 | O | Q | X¹, X², X⁴, X⁵ and | R⁵, R⁶, R⁷, and | —CH₂—CH₂— | CH₃ | H |
| | | | X⁶ are carbon; | R⁹ are H | | | |
| | | | X³ is nitrogen; and | | | | |
| | | | R⁵, R⁶, R⁷ and R⁹ are | | | | |
| | | | hydrogen | | | | |
| 255 | O | Q | X¹, X², X⁴, X⁵ and | R⁵, R⁷, and | lower alkylene, alkenylene, | hydrogen, C1-C2 alkyl, or | hydrogen, C1-C2 |
| | | | X⁶ are carbon; | R⁹ are H, R⁶ is | alkynylene, or acyl or aryl, | C1-C2 alkyl substituted | alkyl, or C1-C2 alkyl |
| | | | X³ is nitrogen; and | methyl | which is optionally substituted | with one or more | substituted with one or |
| | | | R⁵, R⁶, R⁷ and R⁹ are | | with hydroxyl, alkoxyl, amino, | halogen atoms | more halogen atoms |
| | | | hydrogen | | alkylamino, thiol, thioalkyl, or | | |
| | | | | | halogen | | |
| 256 | O | Q | X¹, X², X⁴, X⁵ and | R⁵, R⁷, and | lower alkylene, alkenylene, | hydrogen, C1-C2 alkyl, or | CH₃ or C₂H₅ |
| | | | X⁶ are carbon; | R⁹ are H, R⁶ is | alkynylene, or acyl or aryl, | C1-C2 alkyl substituted | |
| | | | X³ is nitrogen; and | methyl | which is optionally substituted | with one or more | |
| | | | R⁵, R⁶, R⁷ and R⁹ are | | with hydroxyl, alkoxyl, amino, | halogen atoms | |
| | | | hydrogen | | alkylamino, thiol, thioalkyl, or | | |
| | | | | | halogen | | |
| 257 | O | Q | X¹, X², X⁴, X⁵ and | R⁵, R⁷, and | lower alkylene, alkenylene, | hydrogen, C1-C2 alkyl, or | H |
| | | | X⁶ are carbon; | R⁹ are H, R⁶ is | alkynylene, or acyl or aryl, | C1-C2 alkyl substituted | |
| | | | X³ is nitrogen; and | methyl | which is optionally substituted | with one or more | |
| | | | R⁵, R⁶, R⁷ and R⁹ are | | with hydroxyl, alkoxyl, amino, | halogen atoms | |
| | | | hydrogen | | alkylamino, thiol, thioalkyl, or | | |
| | | | | | halogen | | |
| 258 | O | Q | X¹, X², X⁴, X⁵ and | R⁵, R⁷, and | lower alkylene, alkenylene, | CH₃ or C₂H₅ | hydrogen, C1-C2 |
| | | | X⁶ are carbon; | R⁹ are H, R⁶ is | alkynylene, or acyl or aryl, | | alkyl, or C1-C2 alkyl |
| | | | X³ is nitrogen; and | methyl | which is optionally substituted | | substituted with one or |
| | | | R⁵, R⁶, R⁷ and R⁹ are | | with hydroxyl, alkoxyl, amino, | | more halogen atoms |
| | | | hydrogen | | alkylamino, thiol, thioalkyl, or | | |
| | | | | | halogen | | |
| 259 | O | Q | X¹, X², X⁴, X⁵ and | R⁵, R⁷, and | lower alkylene, alkenylene, | CH₃ or C₂H₅ | CH₃ or C₂H₅ |
| | | | X⁶ are carbon; | R⁹ are H, R⁶ is | alkynylene, or acyl or aryl, | | |
| | | | X³ is nitrogen; and | methyl | which is optionally substituted | | |
| | | | R⁵, R⁶, R⁷ and R⁹ are | | with hydroxyl, alkoxyl, amino, | | |
| | | | hydrogen | | alkylamino, thiol, thioalkyl, or | | |
| | | | | | halogen | | |
| 260 | O | Q | X¹, X², X⁴, X⁵ and | R⁵, R⁷, and | lower alkylene, alkenylene, | CH₃ or C₂H₅ | H |
| | | | X⁶ are carbon; | R⁹ are H, R⁶ is | alkynylene, or acyl or aryl, | | |
| | | | X³ is nitrogen; and | methyl | which is optionally substituted | | |
| | | | R⁵, R⁶, R⁷ and R⁹ are | | with hydroxyl, alkoxyl, amino, | | |
| | | | hydrogen | | alkylamino, thiol, thioalkyl, or | | |
| | | | | | halogen | | |
| 261 | O | Q | X¹, X², X⁴, X⁵ and | R⁵, R⁷, and | lower alkylene, alkenylene, | CH₃ | hydrogen, C1-C2 |
| | | | X⁶ are carbon; | R⁹ are H, R⁶ is | alkynylene, or acyl or aryl, | | alkyl, or C1-C2 alkyl |
| | | | X³ is nitrogen; and | methyl | which is optionally substituted | | substituted with one or |
| | | | R⁵, R⁶, R⁷ and R⁹ are | | with hydroxyl, alkoxyl, amino, | | more halogen atoms |
| | | | hydrogen | | alkylamino, thiol, thioalkyl, or | | |
| | | | | | halogen | | |
| 262 | O | Q | X¹, X², X⁴, X⁵ and | R⁵, R⁷, and | lower alkylene, alkenylene, | CH₃ | CH₃ or C₂H₅ |
| | | | X⁶ are carbon; | R⁹ are H, R⁶ is | alkynylene, or acyl or aryl, | | |
| | | | X³ is nitrogen; and | methyl | which is optionally substituted | | |
| | | | R⁵, R⁶, R⁷ and R⁹ are | | with hydroxyl, alkoxyl, amino, | | |
| | | | hydrogen | | alkylamino, thiol, thioalkyl, or | | |
| | | | | | halogen | | |
| 263 | O | Q | X¹, X², X⁴, X⁵ and | R⁵, R⁷, and | lower alkylene, alkenylene, | CH₃ | H |
| | | | X⁶ are carbon; | R⁹ are H, R⁶ is | alkynylene, or acyl or aryl, | | |
| | | | X³ is nitrogen; and | methyl | which is optionally substituted | | |
| | | | R⁵, R⁶, R⁷ and R⁹ are | | with hydroxyl, alkoxyl, amino, | | |
| | | | hydrogen | | alkylamino, thiol, thioalkyl, or | | |
| | | | | | halogen | | |
| 264 | O | Q | X¹, X², X⁴, X⁵ and | R⁵, R⁷, and | —CH₂—CH₂— | hydrogen, C1-C2 alkyl, or | hydrogen, C1-C2 |
| | | | X⁶ are carbon; | R⁹ are H, R⁶ is | | C1-C2 alkyl substituted | alkyl, or C1-C2 alkyl |
| | | | X³ is nitrogen; and | methyl | | with one or more | substituted with one or |
| | | | R⁵, R⁶, R⁷ and R⁹ are | | | halogen atoms | more halogen atoms |
| | | | hydrogen | | | | |
| 265 | O | Q | X¹, X², X⁴, X⁵ and | R⁵, R⁷, and | —CH₂—CH₂— | hydrogen, C1-C2 alkyl, or | CH₃ or C₂H₅ |
| | | | X⁶ are carbon; | R⁹ are H, R⁶ is | | C1-C2 alkyl substituted | |
| | | | X³ is nitrogen; and | methyl | | with one or more | |
| | | | R⁵, R⁶, R⁷ and R⁹ are | | | halogen atoms | |
| | | | hydrogen | | | | |
| 266 | O | Q | X¹, X², X⁴, X⁵ and | R⁵, R⁷, and | —CH₂—CH₂— | hydrogen, C1-C2 alkyl, or | H |
| | | | X⁶ are carbon; | R⁹ are H, R⁶ is | | C1-C2 alkyl substituted | |
| | | | X³ is nitrogen; and | methyl | | with one or more | |
| | | | R⁵, R⁶, R⁷ and R⁹ are | | | halogen atoms | |
| | | | hydrogen | | | | |
| 267 | O | Q | X¹, X², X⁴, X⁵ and | R⁵, R⁷, and | —CH₂—CH₂— | CH₃ or C₂H₅ | hydrogen, C1-C2 |
| | | | X⁶ are carbon; | R⁹ are H, R⁶ is | | | alkyl, or C1-C2 alkyl |
| | | | X³ is nitrogen; and | methyl | | | substituted with one or |
| | | | R⁵, R⁶, R⁷ and R⁹ are | | | | more halogen atoms |
| | | | hydrogen | | | | |
| 268 | O | Q | X¹, X², X⁴, X⁵ and | R⁵, R⁷, and | —CH₂—CH₂— | CH₃ or C₂H₅ | CH₃ or C₂H₅ |
| | | | X⁶ are carbon; | R⁹ are H, R⁶ is | | | |
| | | | X³ is nitrogen; and | methyl | | | |
| | | | R⁵, R⁶, R⁷ and R⁹ are | | | | |
| | | | hydrogen | | | | |
| 269 | O | Q | X¹, X², X⁴, X⁵ and | R⁵, R⁷, and | —CH₂—CH₂— | CH₃ or C₂H₅ | H |
| | | | X⁶ are carbon; | R⁹ are H, R⁶ is | | | |
| | | | X³ is nitrogen; and | methyl | | | |
| | | | R⁵, R⁶, R⁷ and R⁹ are | | | | |
| | | | hydrogen | | | | |
| 270 | O | Q | X¹, X², X⁴, X⁵ and | R⁵, R⁷, and | —CH₂—CH₂— | CH₃ | hydrogen, C1-C2 |
| | | | X⁶ are carbon; | R⁹ are H, R⁶ is | | | alkyl, or C1-C2 alkyl |
| | | | X³ is nitrogen; and | methyl | | | substituted with one or |
| | | | R⁵, R⁶, R⁷ and R⁹ are | | | | more halogen atoms |
| | | | hydrogen | | | | |
| 271 | O | Q | X¹, X², X⁴, X⁵ and | R⁵, R⁷, and | —CH₂—CH₂— | CH₃ | CH₃ or C₂H₅ |
| | | | X⁶ are carbon; | R⁹ are H, R⁶ is | | | |
| | | | X³ is nitrogen; and | methyl | | | |
| | | | R⁵, R⁶, R⁷ and R⁹ are | | | | |
| | | | hydrogen | | | | |
| 272 | O | Q | X¹, X², X⁴, X⁵ and | R⁵, R⁷, and | —CH₂—CH₂— | CH₃ | H |
| | | | X⁶ are carbon; | R⁹ are H, R⁶ is | | | |
| | | | X³ is nitrogen; and | methyl | | | |
| | | | R⁵, R⁶, R⁷ and R⁹ are | | | | |
| | | | hydrogen | | | | |
| 273 | O | Q | one, two or | R⁵, R⁶, R⁷, and | lower alkylene, alkenylene, | hydrogen, C1-C2 alkyl, or | hydrogen, C1-C2 |
| | | | three of X¹, | R⁹ are H | alkynylene, or acyl or aryl, | C1-C2 alkyl substituted | alkyl, or C1-C2 alkyl |
| | | | X³ and X⁵, are | | which is optionally substituted | with one or more | substituted with one or |
| | | | nitrogen, and | | with hydroxyl, alkoxyl, amino, | halogen atoms | more halogen atoms |
| | | | two, one or | | alkylamino, thiol, thioalkyl, or | | |
| | | | none of X¹, | | halogen | | |
| | | | X³ and X⁵, are | | | | |
| | | | carbon | | | | |
| 274 | O | Q | one, two or | R⁵, R⁶, R⁷, and | lower alkylene, alkenylene, | hydrogen, C1-C2 alkyl, or | CH₃ or C₂H₅ |
| | | | three of X¹, | R⁹ are H | alkynylene, or acyl or aryl, | C1-C2 alkyl substituted | |
| | | | X³ and X⁵, are | | which is optionally substituted | with one or more | |
| | | | nitrogen, and | | with hydroxyl, alkoxyl, amino, | halogen atoms | |
| | | | two, one or | | alkylamino, thiol, thioalkyl, or | | |
| | | | none of X¹, | | halogen | | |
| | | | X³ and X⁵, are | | | | |
| | | | carbon | | | | |
| 275 | O | Q | one, two or | R⁵, R⁶, R⁷, and | lower alkylene, alkenylene, | hydrogen, C1-C2 alkyl, or | H |
| | | | three of X¹, | R⁹ are H | alkynylene, or acyl or aryl, | C1-C2 alkyl substituted | |
| | | | X³ and X⁵, are | | which is optionally substituted | with one or more | |
| | | | nitrogen, and | | with hydroxyl, alkoxyl, amino, | halogen atoms | |
| | | | two, one or | | alkylamino, thiol, thioalkyl, or | | |
| | | | none of X¹, | | halogen | | |
| | | | X³ and X⁵, are | | | | |
| | | | carbon | | | | |
| 276 | O | Q | one, two or | R⁵, R⁶, R⁷, and | lower alkylene, alkenylene, | CH₃ or C₂H₅ | hydrogen, C1-C2 |
| | | | three of X¹, | R⁹ are H | alkynylene, or acyl or aryl, | | alkyl, or C1-C2 alkyl |
| | | | X³ and X⁵, are | | which is optionally substituted | | substituted with one or |
| | | | nitrogen, and | | with hydroxyl, alkoxyl, amino, | | more halogen atoms |
| | | | two, one or | | alkylamino, thiol, thioalkyl, or | | |
| | | | none of X¹, | | halogen | | |
| | | | X³ and X⁵, are | | | | |
| | | | carbon | | | | |
| 277 | O | Q | one, two or | R⁵, R⁶, R⁷, and | lower alkylene, alkenylene, | CH₃ or C₂H₅ | CH₃ or C₂H₅ |
| | | | three of X¹, | R⁹ are H | alkynylene, or acyl or aryl, | | |
| | | | X³ and X⁵, are | | which is optionally substituted | | |
| | | | nitrogen, and | | with hydroxyl, alkoxyl, amino, | | |
| | | | two, one or | | alkylamino, thiol, thioalkyl, or | | |
| | | | none of X¹, | | halogen | | |
| | | | X³ and X⁵, are | | | | |
| | | | carbon | | | | |
| 278 | O | Q | one, two or | R⁵, R⁶, R⁷, and | lower alkylene, alkenylene, | CH₃ or C₂H₅ | H |
| | | | three of X¹, | R⁹ are H | alkynylene, or acyl or aryl, | | |
| | | | X³ and X⁵, are | | which is optionally substituted | | |
| | | | nitrogen, and | | with hydroxyl, alkoxyl, amino, | | |
| | | | two, one or | | alkylamino, thiol, thioalkyl, or | | |
| | | | none of X¹, | | halogen | | |
| | | | X³ and X⁵, are | | | | |
| | | | carbon | | | | |
| 279 | O | Q | one, two or | R⁵, R⁶, R⁷, and | lower alkylene, alkenylene, | CH₃ | hydrogen, C1-C2 |
| | | | three of X¹, | R⁹ are H | alkynylene, or acyl or aryl, | | alkyl, or C1-C2 alkyl |
| | | | X³ and X⁵, are | | which is optionally substituted | | substituted with one or |
| | | | nitrogen, and | | with hydroxyl, alkoxyl, amino, | | more halogen atoms |
| | | | two, one or | | alkylamino, thiol, thioalkyl, or | | |
| | | | none of X¹, | | halogen | | |
| | | | X³ and X⁵, are | | | | |
| | | | carbon | | | | |
| 280 | O | Q | one, two or | R⁵, R⁶, R⁷, and | lower alkylene, alkenylene, | CH₃ | CH₃ or C₂H₅ |
| | | | three of X¹, | R⁹ are H | alkynylene, or acyl or aryl, | | |
| | | | X³ and X⁵, are | | which is optionally substituted | | |
| | | | nitrogen, and | | with hydroxyl, alkoxyl, amino, | | |
| | | | two, one or | | alkylamino, thiol, thioalkyl, or | | |
| | | | none of X¹, | | halogen | | |
| | | | X³ and X⁵, are | | | | |
| | | | carbon | | | | |
| 281 | O | Q | one, two or | R⁵, R⁶, R⁷, and | lower alkylene, alkenylene, | CH₃ | H |
| | | | three of X¹, | R⁹ are H | alkynylene, or acyl or aryl, | | |
| | | | X³ and X⁵, are | | which is optionally substituted | | |
| | | | nitrogen, and | | with hydroxyl, alkoxyl, amino, | | |
| | | | two, one or | | alkylamino, thiol, thioalkyl, or | | |
| | | | none of X¹, | | halogen | | |
| | | | X³ and X⁵, are | | | | |
| | | | carbon | | | | |
| 282 | O | Q | one, two or | R⁵, R⁶, R⁷, and | —CH₂—CH₂— | hydrogen, C1-C2 alkyl, or | hydrogen, C1-C2 |
| | | | three of X¹, | R⁹ are H | | C1-C2 alkyl substituted | alkyl, or C1-C2 alkyl |
| | | | X³ and X⁵, are | | | with one or more | substituted with one or |
| | | | nitrogen, and | | | halogen atoms | more halogen atoms |
| | | | two, one or | | | | |
| | | | none of X¹, | | | | |
| | | | X³ and X⁵, are | | | | |
| | | | carbon | | | | |
| 283 | O | Q | one, two or | R⁵, R⁶, R⁷, and | —CH₂—CH₂— | hydrogen, C1-C2 alkyl, or | CH₃ or C₂H₅ |
| | | | three of X¹, | R⁹ are H | | C1-C2 alkyl substituted | |
| | | | X³ and X⁵, are | | | with one or more | |
| | | | nitrogen, and | | | halogen atoms | |
| | | | two, one or | | | | |
| | | | none of X¹, | | | | |
| | | | X³ and X⁵, are | | | | |
| | | | carbon | | | | |
| 284 | O | Q | one, two or | R⁵, R⁶, R⁷, and | —CH₂—CH₂— | hydrogen, C1-C2 alkyl, or | H |
| | | | three of X¹, | R⁹ are H | | C1-C2 alkyl substituted | |
| | | | X³ and X⁵, are | | | with one or more | |
| | | | nitrogen, and | | | halogen atoms | |
| | | | two, one or | | | | |
| | | | none of X¹, | | | | |
| | | | X³ and X⁵, are | | | | |
| | | | carbon | | | | |
| 285 | O | Q | one, two or | R⁵, R⁶, R⁷, and | —CH₂—CH₂— | CH₃ or C₂H₅ | hydrogen, C1-C2 |
| | | | three of X¹, | R⁹ are H | | | alkyl, or C1-C2 alkyl |
| | | | X³ and X⁵, are | | | | substituted with one or |
| | | | nitrogen, and | | | | more halogen atoms |
| | | | two, one or | | | | |
| | | | none of X¹, | | | | |
| | | | X³ and X⁵, are | | | | |
| | | | carbon | | | | |
| 286 | O | Q | one, two or | R⁵, R⁶, R⁷, and | —CH₂—CH₂— | CH₃ or C₂H₅ | CH₃ or C₂H₅ |
| | | | three of X¹, | R⁹ are H | | | |
| | | | X³ and X⁵, are | | | | |
| | | | nitrogen, and | | | | |
| | | | two, one or | | | | |
| | | | none of X¹, | | | | |
| | | | X³ and X⁵, are | | | | |
| | | | carbon | | | | |
| 287 | O | Q | one, two or | R⁵, R⁶, R⁷, and | —CH₂—CH₂— | CH₃ or C₂H₅ | H |
| | | | three of X¹, | R⁹ are H | | | |
| | | | X³ and X⁵, are | | | | |
| | | | nitrogen, and | | | | |
| | | | two, one or | | | | |
| | | | none of X¹, | | | | |
| | | | X³ and X⁵, are | | | | |
| | | | carbon | | | | |
| 288 | O | Q | one, two or | R⁵, R⁶, R⁷, and | —CH₂—CH₂— | CH₃ | hydrogen, C1-C2 |
| | | | three of X¹, | R⁹ are H | | | alkyl, or C1-C2 alkyl |
| | | | X³ and X⁵, are | | | | substituted with one or |
| | | | nitrogen, and | | | | more halogen atoms |
| | | | two, one or | | | | |
| | | | none of X¹, | | | | |
| | | | X³ and X⁵, are | | | | |
| | | | carbon | | | | |
| 289 | O | Q | one, two or | R⁵, R⁶, R⁷, and | —CH₂—CH₂— | CH₃ | CH₃ or C₂H₅ |
| | | | three of X¹, | R⁹ are H | | | |
| | | | X³ and X⁵, are | | | | |
| | | | nitrogen, and | | | | |
| | | | two, one or | | | | |
| | | | none of X¹, | | | | |
| | | | X³ and X⁵, are | | | | |
| | | | carbon | | | | |
| 290 | O | Q | one, two or | R⁵, R⁶, R⁷, and | —CH₂—CH₂— | CH₃ | H |
| | | | three of X¹, | R⁹ are H | | | |
| | | | X³ and X⁵, are | | | | |
| | | | nitrogen, and | | | | |
| | | | two, one or | | | | |
| | | | none of X¹, | | | | |
| | | | X³ and X⁵, are | | | | |
| | | | carbon | | | | |
| 291 | O | Q | one, two or | R⁵, R⁷, and | lower alkylene, alkenylene, | hydrogen, C1-C2 alkyl, or | hydrogen, C1-C2 |
| | | | three of X¹, | R⁹ are H, R⁶ is | alkynylene, or acyl or aryl, | C1-C2 alkyl substituted | alkyl, or C1-C2 alkyl |
| | | | X³ and X⁵, are | methyl | which is optionally substituted | with one or more | substituted with one or |
| | | | nitrogen, and | | with hydroxyl, alkoxyl, amino, | halogen atoms | more halogen atoms |
| | | | two, one or | | alkylamino, thiol, thioalkyl, or | | |
| | | | none of X¹, | | halogen | | |
| | | | X³ and X⁵, are | | | | |
| | | | carbon | | | | |
| 292 | O | Q | one, two or | R⁵, R⁷, and | lower alkylene, alkenylene, | hydrogen, C1-C2 alkyl, or | CH₃ or C₂H₅ |
| | | | three of X¹, | R⁹ are H, R⁶ is | alkynylene, or acyl or aryl, | C1-C2 alkyl substituted | |
| | | | X³ and X⁵, are | methyl | which is optionally substituted | with one or more | |
| | | | nitrogen, and | | with hydroxyl, alkoxyl, amino, | halogen atoms | |
| | | | two, one or | | alkylamino, thiol, thioalkyl, or | | |
| | | | none of X¹, | | halogen | | |
| | | | X³ and X⁵, are | | | | |
| | | | carbon | | | | |
| 293 | O | Q | one, two or | R⁵, R⁷, and | lower alkylene, alkenylene, | hydrogen, C1-C2 alkyl, or | H |
| | | | three of X¹, | R⁹ are H, R⁶ is | alkynylene, or acyl or aryl, | C1-C2 alkyl substituted | |
| | | | X³ and X⁵, are | methyl | which is optionally substituted | with one or more | |
| | | | nitrogen, and | | with hydroxyl, alkoxyl, amino, | halogen atoms | |
| | | | two, one or | | alkylamino, thiol, thioalkyl, or | | |
| | | | none of X¹, | | halogen | | |
| | | | X³ and X⁵, are | | | | |
| | | | carbon | | | | |
| 294 | O | Q | one, two or | R⁵, R⁷, and | lower alkylene, alkenylene, | CH₃ or C₂H₅ | hydrogen, C1-C2 |
| | | | three of X¹, | R⁹ are H, R⁶ is | alkynylene, or acyl or aryl, | | alkyl, or C1-C2 alkyl |
| | | | X³ and X⁵, are | methyl | which is optionally substituted | | substituted with one or |
| | | | nitrogen, and | | with hydroxyl, alkoxyl, amino, | | more halogen atoms |
| | | | two, one or | | alkylamino, thiol, thioalkyl, or | | |
| | | | none of X¹, | | halogen | | |
| | | | X³ and X⁵, are | | | | |
| | | | carbon | | | | |
| 295 | O | Q | one, two or | R⁵, R⁷, and | lower alkylene, alkenylene, | CH₃ or C₂H₅ | CH₃ or C₂H₅ |
| | | | three of X¹, | R⁹ are H, R⁶ is | alkynylene, or acyl or aryl, | | |
| | | | X³ and X⁵, are | methyl | which is optionally substituted | | |
| | | | nitrogen, and | | with hydroxyl, alkoxyl, amino, | | |
| | | | two, one or | | alkylamino, thiol, thioalkyl, or | | |
| | | | none of X¹, | | halogen | | |
| | | | X³ and X⁵, are | | | | |
| | | | carbon | | | | |
| 296 | O | Q | one, two or | R⁵, R⁷, and | lower alkylene, alkenylene, | CH₃ or C₂H₅ | H |
| | | | three of X¹, | R⁹ are H, R⁶ is | alkynylene, or acyl or aryl, | | |
| | | | X³ and X⁵, are | methyl | which is optionally substituted | | |
| | | | nitrogen, and | | with hydroxyl, alkoxyl, amino, | | |
| | | | two, one or | | alkylamino, thiol, thioalkyl, or | | |
| | | | none of X¹, | | halogen | | |
| | | | X³ and X⁵, are | | | | |
| | | | carbon | | | | |
| 297 | O | Q | one, two or | R⁵, R⁷, and | lower alkylene, alkenylene, | CH₃ | hydrogen, C1-C2 |
| | | | three of X¹, | R⁹ are H, R⁶ is | alkynylene, or acyl or aryl, | | alkyl, or C1-C2 alkyl |
| | | | X³ and X⁵, are | methyl | which is optionally substituted | | substituted with one or |
| | | | nitrogen, and | | with hydroxyl, alkoxyl, amino, | | more halogen atoms |
| | | | two, one or | | alkylamino, thiol, thioalkyl, or | | |
| | | | none of X¹, | | halogen | | |
| | | | X³ and X⁵, are | | | | |
| | | | carbon | | | | |
| 298 | O | Q | one, two or | R⁵, R⁷, and | lower alkylene, alkenylene, | CH₃ | CH₃ or C₂H₅ |
| | | | three of X¹, | R⁹ are H, R⁶ is | alkynylene, or acyl or aryl, | | |
| | | | X³ and X⁵, are | methyl | which is optionally substituted | | |
| | | | nitrogen, and | | with hydroxyl, alkoxyl, amino, | | |
| | | | two, one or | | alkylamino, thiol, thioalkyl, or | | |
| | | | none of X¹, | | halogen | | |
| | | | X³ and X⁵, are | | | | |
| | | | carbon | | | | |
| 299 | O | Q | one, two or | R⁵, R⁷, and | lower alkylene, alkenylene, | CH₃ | H |
| | | | three of X¹, | R⁹ are H, R⁶ is | alkynylene, or acyl or aryl, | | |
| | | | X³ and X⁵, are | methyl | which is optionally substituted | | |
| | | | nitrogen, and | | with hydroxyl, alkoxyl, amino, | | |
| | | | two, one or | | alkylamino, thiol, thioalkyl, or | | |
| | | | none of X¹, | | halogen | | |
| | | | X³ and X⁵, are | | | | |
| | | | carbon | | | | |
| 300 | O | Q | one, two or | R⁵, R⁷, and | —CH₂—CH₂— | hydrogen, C1-C2 alkyl, or | hydrogen, C1-C2 |
| | | | three of X¹, | R⁹ are H, R⁶ is | | C1-C2 alkyl substituted | alkyl, or C1-C2 alkyl |
| | | | X³ and X⁵, are | methyl | | with one or more | substituted with one or |
| | | | nitrogen, and | | | halogen atoms | more halogen atoms |
| | | | two, one or | | | | |
| | | | none of X¹, | | | | |
| | | | X³ and X⁵, are | | | | |
| | | | carbon | | | | |
| 301 | O | Q | one, two or | R⁵, R⁷, and | —CH₂—CH₂— | hydrogen, C1-C2 alkyl, or | CH₃ or C₂H₅ |
| | | | three of X¹, | R⁹ are H, R⁶ is | | C1-C2 alkyl substituted | |
| | | | X³ and X⁵, are | methyl | | with one or more | |
| | | | nitrogen, and | | | halogen atoms | |
| | | | two, one or | | | | |
| | | | none of X¹, | | | | |
| | | | X³ and X⁵, are | | | | |
| | | | carbon | | | | |
| 302 | O | Q | one, two or | R⁵, R⁷, and | —CH₂—CH₂— | hydrogen, C1-C2 alkyl, or | H |
| | | | three of X¹, | R⁹ are H, R⁶ is | | C1-C2 alkyl substituted | |
| | | | X³ and X⁵, are | methyl | | with one or more | |
| | | | nitrogen, and | | | halogen atoms | |
| | | | two, one or | | | | |
| | | | none of X¹, | | | | |
| | | | X³ and X⁵, are | | | | |
| | | | carbon | | | | |
| 303 | O | Q | one, two or | R⁵, R⁷, and | —CH₂—CH₂— | CH₃ or C₂H₅ | hydrogen, C1-C2 |
| | | | three of X¹, | R⁹ are H, R⁶ is | | | alkyl, or C1-C2 alkyl |
| | | | X³ and X⁵, are | methyl | | | substituted with one or |
| | | | nitrogen, and | | | | more halogen atoms |
| | | | two, one or | | | | |
| | | | none of X¹, | | | | |
| | | | X³ and X⁵, are | | | | |
| | | | carbon | | | | |
| 304 | O | Q | one, two or | R⁵, R⁷, and | —CH₂—CH₂— | CH₃ or C₂H₅ | CH₃ or C₂H₅ |
| | | | three of X¹, | R⁹ are H, R⁶ is | | | |
| | | | X³ and X⁵, are | methyl | | | |
| | | | nitrogen, and | | | | |
| | | | two, one or | | | | |
| | | | none of X¹, | | | | |
| | | | X³ and X⁵, are | | | | |
| | | | carbon | | | | |
| 305 | O | Q | one, two or | R⁵, R⁷, and | —CH₂—CH₂— | CH₃ or C₂H₅ | H |
| | | | three of X¹, | R⁹ are H, R⁶ is | | | |
| | | | X³ and X⁵, are | methyl | | | |
| | | | nitrogen, and | | | | |
| | | | two, one or | | | | |
| | | | none of X¹, | | | | |
| | | | X³ and X⁵, are | | | | |
| | | | carbon | | | | |
| 306 | O | Q | one, two or | R⁵, R⁷, and | —CH₂—CH₂— | CH₃ | hydrogen, C1-C2 |
| | | | three of X¹, | R⁹ are H, R⁶ is | | | alkyl, or C1-C2 alkyl |
| | | | X³ and X⁵, are | methyl | | | substituted with one or |
| | | | nitrogen, and | | | | more halogen atoms |
| | | | two, one or | | | | |
| | | | none of X¹, | | | | |
| | | | X³ and X⁵, are | | | | |
| | | | carbon | | | | |
| 307 | O | Q | one, two or | R⁵, R⁷, and | —CH₂—CH₂— | CH₃ | CH₃ or C₂H₅ |
| | | | three of X¹, | R⁹ are H, R⁶ is | | | |
| | | | X³ and X⁵, are | methyl | | | |
| | | | nitrogen, and | | | | |
| | | | two, one or | | | | |
| | | | none of X¹, | | | | |
| | | | X³ and X⁵, are | | | | |
| | | | carbon | | | | |
| 308 | O | Q | one, two or | R⁵, R⁷, and | —CH₂—CH₂— | CH₃ | H |
| | | | three of X¹, | R⁹ are H, R⁶ is | | | |
| | | | X³ and X⁵, are | methyl | | | |
| | | | nitrogen, and | | | | |
| | | | two, one or | | | | |
| | | | none of X¹, | | | | |
| | | | X³ and X⁵, are | | | | |
| | | | carbon | | | | |

In some embodiments, the compound is a pharmaceutically acceptable salt comprising at least one counter ion chosen from phosphate, hydrochloride, besylate, benzoate, carbonate, chloride, citrate, dihydrochloride, dimaleate, diphosphate, estolate, fumarate, gluconate, malate, maleate, pamoate, stearate, succinate, sulfate, sulfonate, tartrate, tosylate, and valerate. In some embodiments, the counter ion is phosphate. In some embodiments, the counter ion is hydrochloride.

In the context of a chemical structure, a reference to a chemical structure, or a structure provided as part of a synthetic scheme, a number or letter normally designated as a superscript, for example, the “1” in R¹, or the “L” in R_{L}, may be referred to as a subscript, for example, R₁ or R_{L}, or without any modification of script, such as, for example, R1 or R_{L}.

Any definition herein may be used in combination with any other definition to describe a composite structural group. By convention, the trailing element of any such definition is that which attaches to a parent moiety. The composite group alkylamido, for example, would represent an alkyl group attached to a parent molecule through an amido group, and the term alkoxyalkyl would represent an alkoxy group attached to a parent molecule through an alkyl group, for example.

When a group is defined to be “null,” the group is absent. The term “optionally substituted” means the anteceding group may be substituted or unsubstituted. The term “substituted,” as used herein, refers, without limitation, to one or more substituents which can include, for example, substituents independently selected from the following groups or a particular designated set of groups, alone or in combination: lower alkyl, lower alkenyl, lower alkynyl, lower alkanoyl, lower heteroalkyl, lower aryl, lower cycloalkyl, lower heteroaryl, lower heterocycloalkyl, aryl, cycloalkyl, heteroaryl, heterocycloalkyl, lower haloalkyl, lower haloalkenyl, lower haloalkynyl, lower perhaloalkyl, lower perhaloalkoxy, phenyl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic, heterocyclic aryl, or heteroaryl ring system having zero to three heteroatoms, for example, forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed. An optional substitution often is as defined, sometimes immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Certain groups may be attached to a parent molecule or may occupy a position in a chain of elements from either end as written. Thus, by way of example only, an asymmetrical group such as —C(O)N(R)— may be attached to a parent moiety at either the carbon or the nitrogen.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. Non-limiting examples of acyl groups include formyl, alkanoyl and aroyl.

An “acetyl” group refers to a —C(O)CH₃ group.

The term “aliphatic,” as used herein, refers to saturated and partially unsaturated, nonaromatic, straight chain (i.e., unbranched), branched and cyclic (including bicyclic and polycyclic) hydrocarbons which may be optionally substituted with one or more functional groups. In certain embodiments, an aliphatic group contains 1 to 20 carbon atoms, 1 to 10 carbon atoms, 1 to 8 carbon atoms, 1 to 6 carbon atoms or 1 to 3 carbon atoms.

An “alkylcarbonyl” or “alkanoyl” group refers to an alkyl group attached to a parent molecular moiety through a carbonyl group. Non-limiting examples of such groups include methylcarbonyl and ethylcarbonyl.

The term “alkenyl,” as used herein, alone or in combination, refers to a straight-chain or branched-chain hydrocarbon radical having one or more double bonds and containing from 2 to 20 carbon atoms. In certain embodiments, an alkenyl includes 2 to 6 carbon atoms. The term “alkenylene” refers to a carbon-carbon double bond system attached at two or more positions such as ethenylene [(—CH═CH—),(—C::C—)]. Non-limiting examples of alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, where the term alkyl is as defined below. Non-limiting examples of alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a saturated straight-chain or branched-chain hydrocarbon radical containing from 1 to 20 carbon atoms. The term “straight-chain alkyl” refers to a saturated straight-chain hydrocarbon radical. The term “branched-chain alkyl” refers to a saturated branched-chain hydrocarbon radical. In certain embodiments, an alkyl includes 1 to 10 carbon atoms, 1 to 8 carbon atoms, 1 to 6 carbon atoms or 1 to 3 carbon atoms. Alkyl groups may be optionally substituted as defined herein. Non-limiting examples of alkyl radicals include methyl, ethyl, n-propyl, isopropyl, n-butyl, isobutyl, sec-butyl, tert-butyl, pentyl, iso-amyl, hexyl, octyl, nonyl and the like.

The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to a parent molecular moiety through an amino group. Alkylamino groups include mono- or dialkylated groups, non-limiting examples of which include N-methylamino, N-ethylamino, N,N— dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical where the term alkyl is as defined above and where the sulfur may be singly or doubly oxidized. Non-limiting examples of alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, an alkynyl includes 2 to 6 carbon atoms. In some embodiments, an alkynyl includes 2 to 4 carbon atoms. The term “alkynylene” refers to a carbon-carbon triple bond attached at two positions such as ethynylene (—C:::C—, —C≡C—). Non-limiting examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to a parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, includes an acyl group attached to a parent moiety through an amino group. A non-limiting example of an “acylamino” group is acetylamino (CH₃C(O)NH—).

The term “amino,” as used herein, alone or in combination, refers to —NRR′, where R and R′ are independently chosen from hydrogen, alkyl, alkenyl, alkynyl, acyl, heteroalkyl, aryl, cycloalkyl, heteroaryl, and heterocycloalkyl, any of which may themselves be optionally substituted. Additionally, R and R′ may combine to form heterocycloalkyl or heteroaryl, either of which may be optionally substituted.

The term “aryl,” as used herein, alone or in combination, refers to an aromatic cyclic ring system, or aromatic hydrocarbon ring system, in which all of the atoms that form the covalent structure of the one or more aromatic rings are carbon (referred to herein as an “aryl ring”). The aryl ring may be optionally substituted as defined herein. The ring system may be monocyclic or fused polycyclic, for example, bicyclic or tricylic (containing two or three rings fused together). In certain embodiments, the monocyclic aryl ring is C4-C10, or C5-C9, or C5-C8, or C5-C7, or, in certain embodiments, C5-C6, where these carbon numbers refer to the number of carbon ring member atoms that form the ring system. In some embodiments, the polycyclic ring system is a bicyclic aryl group, where the bicyclic aryl group in some embodiments is C8-C12, or, for example, C9-C10. In some embodiments, the polycyclic ring system is a tricyclic aryl group, where the tricyclic aryl group is C11-C18, or, for example, C12-C16. Non-limiting examples of aryl ring systems include phenyl (monocyclic, C6), naphthyl (bicyclic, 010), anthracenyl (tricyclic, C14) and phenanthryl (tricyclic, C14).

The term “arylalkenyl” or “aralkenyl,” as used herein, alone or in combination, refers to an aryl group attached to a parent molecular moiety through an alkenyl group.

The term “arylalkoxy” or “aralkoxy,” as used herein, alone or in combination, refers to an aryl group attached to a parent molecular moiety through an alkoxy group.

The term “arylalkyl” or “aralkyl,” as used herein, alone or in combination, refers to an aryl group attached to a parent molecular moiety through an alkyl group.

The term “arylalkynyl” or “aralkynyl,” as used herein, alone or in combination, refers to an aryl group attached to a parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid, non-limiting examples of which include benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term “aryloxy” as used herein, alone or in combination, refers to an aryl group attached to a parent molecular moiety through an oxy.

The terms “benzo” and “benz,” as used herein, alone or in combination, refer to the divalent radical C₆H₄═ derived from benzene. Non-limiting examples include benzothiophene and benzimidazole.

The term “carbamate,” as used herein, alone or in combination, refers to an ester of carbamic acid (—NHCOO—) which may be attached to a parent molecular moiety from either the nitrogen or acid end, and which may be optionally substituted as defined herein.

The term “O-carbamyl” as used herein, alone or in combination, refers to a —OC(O)NRR′ group where R and R′ are as defined herein.

The term “N-carbamyl” as used herein, alone or in combination, refers to a ROC(O)NR′— group, where R and R′ are defined herein.

The term “carbonyl,” as used herein, when alone includes formyl [—C(O)H] and in combination includes a —C(O)— group.

The term “carboxyl” or “carboxy,” as used herein, refers to —C(O)OH or the corresponding “carboxylate” anion (e.g., in a carboxylic acid salt). An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The terms “cycloalkyl,” and, interchangeably, “carbocycle,” as used herein, alone or in combination, refers to a ring system in which all of the ring member atoms are carbon and at least one of the rings is a saturated or partially unsaturated aliphatic cyclic ring moiety (referred to herein as a “cycloalkyl ring” or “carbocycle ring”). In some embodiments, each cyclic moiety contains from 3 to 12 carbon ring member atoms which may be optionally substituted as defined herein. In some embodiments, a cycloalkyl group contains 3 to 10 carbon ring member atoms. In certain embodiments, a cycloalkyl includes 5 to 7 carbon atoms. In certain embodiments, a cycloalkyl includes 5 to 6 carbon atoms. A cycloalkyl can be a monocyclic or polycyclic, e.g., bicyclic or tricyclic, ring system in which at least one cyclic ring is a cycloalkyl ring. In certain embodiments, the monocyclic cycloalkyl ring is C3-C10, or C5-C9, or C5-C8, or C5-C7, or, in certain embodiments, C5-C6, where these carbon numbers refer to the number of carbon ring member atoms that form the ring system. Polycyclic cycloalkyl ring systems include fused, bridged and spiro-fused rings. Polycyclic cycloalkyl ring systems as defined herein, include ring systems in which one or more cycloalkyl rings is/are fused to one or more aryl rings (benzo-fused cycloalkyl ring systems) and/or other cycloalkyl rings. In some embodiments, all of the rings in a polycyclic cycloalkyl ring system are cycloalkyl rings. In some embodiments, the polycyclic ring system is a bicyclic cycloalkyl group, where the bicyclic cycloalkyl group in some embodiments is C8-C12, or, for example, C9-C10. In some embodiments, the polycyclic ring system is a tricyclic cycloalkyl group, where the tricyclic cycloalkyl group is C11-C18, or, for example, C12-C16. Non-limiting examples of such cycloalkyl groups include cyclopropyl, cyclobutyl, cyclopentyl, cyclohexyl, cycloheptyl, adamantyl, octahydronaphthalene, decahydronaphthalene, bicyclo[1,1,1]pentane and the like. Examples of aryl-fused cyclolalkyl ring systems include a benzene ring fused to hydrogenated or partially hydrogenated ring systems, non-limiting examples of which include dihydronaphthalene, tetrahydronaphthalene and indanyl. In polycyclic systems in which a cycloalkyl is fused to an aryl, attachment of the polycycle to the indicated point of attachment on the parent molecule may be through any ring atom of the polycycle rings. In some embodiments of polycyclic cycloalkyls, the polycycle is attached to the indicated point of attachment through a ring member atom of a cycloalkyl ring. In some embodiments of polycyclic cycloalkyls, the polycycle is attached to the indicated point of attachment through a ring member atom of a ring that is not a cycloalkyl ring, e.g., an aryl ring.

The term “carbocycle-alkyl” or “cycloalkylalkyl” as used herein, alone or in combination, refers to a carbocycle group attached to a parent molecular moiety through an alkyl group.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to a parent molecular moiety through an oxygen atom.

The term “haloalkyl,” as used herein, alone or in combination, refers to an alkyl radical having the meaning as defined above where one or more hydrogens are replaced with a halogen. Specifically included are monohaloalkyl, dihaloalkyl and polyhaloalkyl radicals. A monohaloalkyl radical, for example, sometimes include an iodo, bromo, chloro or fluoro atom within the radical. Dihalo and polyhaloalkyl radicals sometimes include two or more of the same halo atoms or a combination of different halo radicals. Non-limiting examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Non-limiting examples include fluoromethylene (—CFH—), difluoromethylene (—CF₂—), chloromethylene (—CHCl—) and the like.

The term “heteroaliphatic,” as used herein, refers to an aliphatic moiety, as defined herein, that contains one or more heteroatoms, such as, for example, oxygen, nitrogen, sulfur, phosphorous and/or silicon, e.g., in place of a carbon atom or between carbon atoms. In some embodiments, a heteroaliphatic group contains from one to three heteroatoms chosen from O, N, and S, and where the nitrogen and sulfur atoms may optionally be oxidized and the nitrogen heteroatom may optionally be quaternized. In certain embodiments, the heteroatom(s) may be placed at any interior position of the heteroaliphatic group. In some embodiments, up to two heteroatoms may be consecutive. In certain embodiments, a heteroaliphatic group includes 2 to 20 carbon atoms, 2 to 10 carbon atoms, 2 to 8 carbon atoms or 2 to 6 carbon atoms.

The term “heteroalkyl,” as used herein, alone or in combination, refers to a saturated or unsaturated, stable straight or branched hydrocarbon chain having the stated number of carbon atoms and one or more heteroatoms, such as, for example, oxygen, nitrogen, sulfur, phosphorous and/or silicon, e.g., in place of a carbon atom. In some embodiments, a heteroalkyl contains from one to three heteroatoms chosen from O, N, and S, and where the nitrogen and sulfur atoms may optionally be oxidized and the nitrogen heteroatom may optionally be quaternized. In certain embodiments, the heteroatom(s) may be placed at any interior position of the heteroalkyl group. In some embodiments, up to two heteroatoms may be consecutive, such as, for example, —CH₂—NH—OCH₃. In certain embodiments, a heteroalkyl includes 2 to 20 carbon atoms, 2 to 10 carbon atoms, 2 to 8 carbon atoms or 2 to 6 carbon atoms. In some instances, a heteroalkyl contains from 1 to 3 degrees of unsaturation. Heteroalkyl groups may be optionally substituted as defined herein.

The term “heteroalkenyl,” as used herein, alone or in combination, refers to an alkenyl moiety, as defined herein, that contains one or more heteroatoms, such as, for example, oxygen, nitrogen, sulfur, phosphorous and/or silicon, e.g., in place of a carbon atom or between carbon atoms. In some embodiments, a heteroalkenyl contains from one to three heteroatoms chosen from O, N, and S, and where the nitrogen and sulfur atoms may optionally be oxidized and the nitrogen heteroatom may optionally be quaternized. In certain embodiments, the heteroatom(s) may be placed at any interior position of the heteroalkenyl group. In some embodiments, up to two heteroatoms may be consecutive. In certain embodiments, a heteroalkenyl includes 2 to 20 carbon atoms, 2 to 10 carbon atoms, 2 to 8 carbon atoms or 2 to 6 carbon atoms.

The term “heteroalkynyl,” as used herein, alone or in combination, refers to an alkynyl moiety, as defined herein, that contains one or more heteroatoms, such as, for example, oxygen, nitrogen, sulfur, phosphorous and/or silicon, e.g., in place of a carbon atom or between carbon atoms. In some embodiments, a heteroalkynyl contains from one to three heteroatoms chosen from O, N, and S, and where the nitrogen and sulfur atoms may optionally be oxidized and the nitrogen heteroatom may optionally be quaternized. In certain embodiments, the heteroatom(s) may be placed at any interior position of the heteroalkynyl group. In some embodiments, up to two heteroatoms may be consecutive. In certain embodiments, a heteroalkynyl includes 2 to 20 carbon atoms, 2 to 10 carbon atoms, 2 to 8 carbon atoms or 2 to 6 carbon atoms.

The term “heteroaryl,” as used herein, alone or in combination, refers to a cyclic ring system in which at least one of the rings is an aromatic ring in which all ring member atoms are carbon, except for at least one heteroatom (referred to herein as a “heteroaryl ring”), such as, for example, nitrogen, oxygen and sulfur. The heteroaryl ring may be optionally substituted as defined herein. A heteroaryl can be a monocyclic or a fused polycyclic, e.g., bicyclic or tricyclic, ring system in which at least one cyclic ring is an aromatic heteroaryl ring. Polycyclic, e.g., bicyclic and tricyclic, fused heteroaryl ring systems as defined herein include heteroaryl ring systems in which one or more heteroaryl rings is/are fused to one or more aryl rings (which are referred to herein as aryl-fused heteroaryl rings), one or more cycloalkyl rings and/or one or more other heteroaryl rings. In some embodiments, all of the rings in a polycyclic heteroaryl ring system are heteroaryl rings. In certain embodiments, a heteroaryl ring contains at least one atom chosen from O, S, and N. In certain embodiments, a heteroaryl ring is a 3 to 15 membered monocyclic ring. In certain embodiments, a monocyclic heteroaryl group may contain from 4 to 10 ring member atoms, and may have, for example, 1 to 4 heteroatoms in the ring, where the remaining ring member atoms are carbon. In some embodiments, a bicyclic heteroaryl ring may contain from 8 to 15 ring member atoms, and have from 1 to 8 heteroatoms, where the remaining ring member atoms are carbon. In some embodiments, a tricyclic heteroaryl ring may contain from 11 to 18 ring member atoms, and have from 1 to 10 heteroatoms, where the remaining ring member atoms are carbon. Non-limiting examples of heteroaryls include pyrrolyl, pyrrolinyl, imidazolyl, pyrazolyl, pyridyl, pyrimidinyl, pyrazinyl, pyridazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, thienopyridinyl, furopyridinyl, pyrrolopyridinyl and the like. Exemplary bicyclic and tricyclic heteroaryl groups include phenanthrolinyl, dibenzofuranyl, acridinyl, phenanthridinyl, dihydro[1,3]oxazolo[4,5-b]pyridinyl, benzothiazolyl, and the like. In polycyclic systems in which a heteroaryl is fused to one or more rings that are not heteroaryl, attachment of the polycycle to the indicated point of attachment on the parent molecule may be through any ring member atom of the polycycle rings. In some embodiments of polycyclic heteroaryls, the polycycle is attached to the indicated point of attachment through a ring member atom of a heteroaryl ring.

In some embodiments of monocyclic or polycyclic heteroaryls, the monocyle or polycycle is attached to the indicated point of attachment through a ring member heteroatom of a heteroaryl ring. In some embodiments of polycyclic heteroaryls, the polycycle is attached to the indicated point of attachment through a ring member atom of a ring that is not a heteroaryl ring, e.g., an aryl ring or a cycloalkyl ring. “Heteroaryl” includes sulfones, sulfoxides, N-oxides of tertiary nitrogen ring member atoms, and carbocyclic fused and benzo-fused ring systems. Non-limiting examples of a heteroaryl group may be referred to as an aryl group having one or more carbon atoms substituted with O, NRⁿ, S, SO, SO₂, where “n” denotes any positive integer.

The term “heteroarylalkyl” as used herein, alone or in combination, refers to an unsubstituted or substituted heteroaryl group attached to a parent molecular moiety through an alkyl group.

The term “heterocycle-alkyl” as used herein, alone or in combination, refers to a substituted or unsubstituted heterocycle group attached to a parent molecular moiety through an alkyl group.

The terms “heterocycloalkyl” and, interchangeably, “heterocycle,” or “heterocyclic” as used herein, alone or in combination, each refer to a ring system in which at least one of the rings is a saturated or partially unsaturated, heteroaliphatic, nonaromatic cyclic ring moiety in which all of the ring member atoms are carbon, except for at least one heteroatom (referred to herein as a “heterocycloalkyl ring,” “heterocycle ring” or “heterocyclic ring”). The one or more heteroatoms that can be in the ring include, for example, nitrogen, oxygen, sulfur, phosphorous and/or silicon. In some embodiments, the ring heteroatom or heteroatoms is selected from nitrogen, oxygen and sulfur. The heterocycloalkyl ring may be optionally substituted as defined herein. A heterocycloalkyl is a monocyclic or polycyclic, e.g., bicyclic or tricyclic, ring system in which at least one cyclic ring is a heterocycloalkyl ring. Polycyclic heterocycloalkyl ring systems include fused, bridged and spiro-fused rings. Polycyclic heterocycloalkyl ring systems as defined herein, include ring systems in which one or more heterocycloalkyl rings is/are fused to one or more cycloalkyl, aryl, heteroaryl and/or heterocycloalkyl rings. In some embodiments, all of the rings in a polycyclic heterocycloalkyl ring system are heterocycloalkyl rings. In certain embodiments, a hetercycloalkyl includes 1 to 4 heteroatoms as ring member atoms. In some embodiments, a hetercycloalkyl moiety includes 1 to 2 heteroatoms as ring member atoms. In certain embodiments, a hetercycloalkyl moiety includes 3 to 8 ring member atoms in each ring. In some embodiments, a hetercycloalkyl moiety includes 3 to 7 ring member atoms in each ring. In yet some embodiments, a hetercycloalkyl moiety includes 5 to 6 ring member atoms in each ring. In some embodiments, a heterocycloalkyl can be a 3 to 15 membered nonaromatic ring, or a fused bicyclic, or tricyclic non-aromatic ring, which contains at least one atom chosen from O, S, and N. In certain embodiments, a monocyclic heterocycloalkyl or heterocycle group may contain from 4 to 10 ring member atoms, and may have, for example, 1 to 4 heteroatoms in the ring, where the remaining ring member atoms are carbon. In some embodiments, a bicyclic heterocycloalkyl or heterocycle group may contain from 8 to 15 ring member atoms, and have from 1 to 8 heteroatoms, where the remaining ring member atoms are carbon. In some embodiments, a tricyclic heterocycloalkyl or heterocycle group may contain from 11 to 18 ring member atoms, and have from 1 to 10 heteroatoms, where the remaining ring member atoms are carbon. The term also includes fused polycyclic groups where one or more heterocyclic rings are fused with one or more cycloalkyl rings, aryl, heteroaryl and/or other heterocyclic groups. In polycyclic systems in which a heterocycloalkyl ring is fused to one or more rings that are not heterocycloalkyl, attachment of the polycycle to the indicated point of attachment on the parent molecule may be through any ring member atom of the polycycle rings. In some embodiments of polycyclic heterocycloalkyls, the polycycle is attached to the indicated point of attachment through a ring member atom of a heterocycloalkyl ring. In some embodiments of monocyclic or polycyclic heterocycloalkyls, the monocyle or polycycle is attached to the indicated point of attachment through a ring member heteroatom of a heterocycloalkyl ring. In some embodiments of polycyclic heterocycloalkyls, the polycycle is attached to the indicated point of attachment through a ring member atom of a ring that is not a heterocycloalkyl ring, e.g., an aryl ring, heteroaryl ring or a cycloalkyl ring. “Heterocycloalkyl” and “heterocycle” include sulfones, sulfoxides and N-oxides of tertiary nitrogen ring member atoms. Non-limiting examples of heterocycle groups include aziridinyl, azetidinyl, 1,3-dioxanyl, 1,4-dioxanyl, 1,3-dioxolanyl, morpholinyl, piperazinyl, pyrrolidinyl, piperidinyl, thiomorpholinyl, pyranyl, dihydropyridinyl, tetrahydropyridinyl, carabazolyl, xanthenyl, 1,3-benzodioxolyl, dihydroisoquinolinyl, dihydrocinnolinyl, dihydrobenzodioxinyl, isoindolinyl, dihydroisoindolyl and dihydroindolyl, and the like. The heterocycle groups may be optionally substituted unless specifically prohibited. Non-limiting examples of heterocycloalkyl groups may be referred to as cycloalkyl group having one or more carbon atoms substituted with O, NRⁿ, S, SO, SO₂, where n denotes any positive integer.

The term “hydrazinyl” as used herein, alone or in combination, refers to two amino groups joined by a single bond, i.e., —HN—NH—.

The term “hydroxy,” as used herein, alone or in combination, refers to —OH.

The term “hydroxyalkyl,” as used herein, alone or in combination, refers to a hydroxy group attached to a parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means a moiety containing from 1 to and including 6 carbon atoms. A “lower alkyl,” for example, refers to an alkyl containing 1 to 6 carbon atoms, 1 to 5 carbon atoms, 1 to 4 carbon atoms, 1 to 3 carbon atoms, or 1 to 2 carbon atoms (e.g., an alkyl containing 1, 2, 3, 4, 5 or 6 carbon atoms).

The term “lower aryl,” as used herein, alone or in combination, means a C4-C6 aryl group, for example, a C5-C6 aryl group. A lower aryl group sometimes is a C4-C6 aryl ring group, or C5-C6 aryl ring group for example, including without limitation, phenyl. The term may also refer to a C8-C10 bicyclic ring aryl group, for example, including without limitation, napthyl. Lower aryl groups, including phenyl or napthyl, may be optionally substituted as provided.

The term “lower heteroaryl,” as used herein, alone or in combination, means a four-membered, five-membered, or six-membered heteroaryl group. A lower heteroaryl group sometimes is (1) a monocyclic heteroaryl ring comprising five or six ring member atoms, of which between one and four of the ring member atoms may be heteroatoms chosen from O, S, and N, or (2) a bicyclic heteroaryl ring, where each of the fused rings comprises five or six ring member atoms, comprising between them one to four heteroatoms chosen from O, S, and N. Lower heteroaryl groups may be optionally substituted as provided.

The term “lower cycloalkyl,” as used herein, alone or in combination, means a monocyclic cycloalkyl having between three and six ring member atoms. Non-limiting examples of lower cycloalkyl include cyclopropyl, cyclobutyl, cyclopentyl, and cyclohexyl. Lower cycloalkyl groups may be optionally substituted as provided.

The term “lower heterocycloalkyl,” as used herein, alone or in combination, means a monocyclic heterocycloalkyl having between three and six ring member atoms, of which between one and four may be heteroatoms chosen from O, S, and N. Non-limiting examples of lower heterocycloalkyls include pyrrolidinyl, imidazolidinyl, pyrazolidinyl, piperidinyl, piperazinyl, and morpholinyl. Lower heterocycloalkyl groups may be optionally substituted as provided.

The term “lower amino,” as used herein, alone or in combination, refers to —NRR′, where R and R′ are independently chosen from hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The terms “mercaptyl” or “mercaptan” as used herein, alone or in combination, refers to an RS— group, where R is as defined herein.

The term “menthol,” as used herein, refers to 2-isopropyl-5-methylcyclohexanol. Menthol contains 3 chiral carbons and the term “menthol” encompasses all stereoisomers of the molecule unless specifically stated otherwise herein. For example, isomers of menthol include the (−)-menthol isomer ((1R, 2S, 5R)-2-isopropyl-5-methylcyclohexanol), (+)-menthol isomer ((1S, 2R, 5S)-2-isopropyl-5-methylcyclohexanol), (−)-isomenthol isomer ((1R, 2S, 5S)-2-isopropyl-5-methylcyclohexanol), (+)-isomenthol isomer ((1S, 2R, 5R)-2-isopropyl-5-methylcyclohexanol), (−)-neomenthol isomer ((1R, 2R, 5S)-2-isopropyl-5-methylcyclohexanol), (+)-neomenthol isomer ((1S, 2S, 5R)-2-isopropyl-5-methylcyclohexanol), (−)-neoisomenthol isomer ((1S, 2S, 5S)-2-isopropyl-5-methylcyclohexanol) and (+)-neoisomenthol isomer ((1R, 2R, 5R)-2-isopropyl-5-methylcyclohexanol).

The term “menthyl,” as used herein, refers to a radical derived from menthol. Typically, a menthyl radical can be linked to another chemical group through the oxygen atom of the menthyl group.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “partially unsaturated,” as used herein, alone or in combination, refers to a straight-chain, branched-chain or ring moiety that includes at least one double or triple bond and that is not fully saturated. The term “partially unsaturated” when used in reference to a ring moiety means a ring having one or multiple sites of unsaturation but does not include aryl rings or heteroaryl rings as defined herein.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “piperitol,” as used herein, refers to p-menth-1-en-3-ol. Piperitol contains 2 chiral carbons and the term “piperitol” encompasses all stereoisomers of the molecule unless specifically stated otherwise herein. For example, isomers of piperitol include (3R, 4R)-piperitol (also referred to as trans-piperitol) and (3S, 4R)-piperitol (also referred to as cis-piperitol).

The term “ring member atoms,” as used herein, refers to all of the atoms that form the covalent structure of a cyclic ring structure.

By “saturated” is meant that the carbon-containing group contains no carbon-carbon double or triple bonds.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether where the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Non-limiting examples include trimethylsilyl, tert-butyldimethylsilyl, triphenylsilyl and the like.

The term “ureido,” as used herein, alone or in combination, refers to the univalent radical NH₂CONH— derived from urea. Non-limiting examples include ureidoproprionate and ureidosuccinate.

Asymmetric centers exist in the compounds disclosed herein. These centers are designated by the symbols “R” or “S,” depending on the configuration of substituents around the chiral carbon atom. Included for compounds herein are all stereochemical isomeric forms, including diastereomeric, enantiomeric, and epimeric forms, as well as d-isomers and 1-isomers, and mixtures thereof, unless otherwise specified. Individual stereoisomers of compounds can be prepared synthetically from commercially available starting materials that contain chiral centers. Individual stereoisomers of compounds can be generated by preparing mixtures of enantiomeric products followed by separation, non-limiting examples of which include conversion to a mixture of diastereomers followed by separation or recrystallization, chromatographic techniques, direct separation of enantiomers on chiral chromatographic columns, or any other appropriate method known in the art. Starting compounds of particular stereochemistry are commercially available or can be made and resolved by techniques known in the art. Additionally, compounds disclosed herein may exist as geometric isomers. Included for compounds herein are all cis, trans, syn, anti, entgegen (E), and zusammen (Z) isomers as well as the appropriate mixtures thereof. Additionally, compounds may exist as tautomers; all tautomeric isomers are included herein unless otherwise specified. Additionally, the compounds disclosed herein can exist in unsolvated as well as solvated forms with pharmaceutically acceptable solvents, non-limiting examples of which include water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

### Synthesis of Multimeric Compounds

Compounds of Formulas I and II may be synthesized following, for example, the following synthesis methods. Methods 1, 2, and 3 are examples of methods that may be used to synthesize compounds of Formula I or II, where Y is L or M.

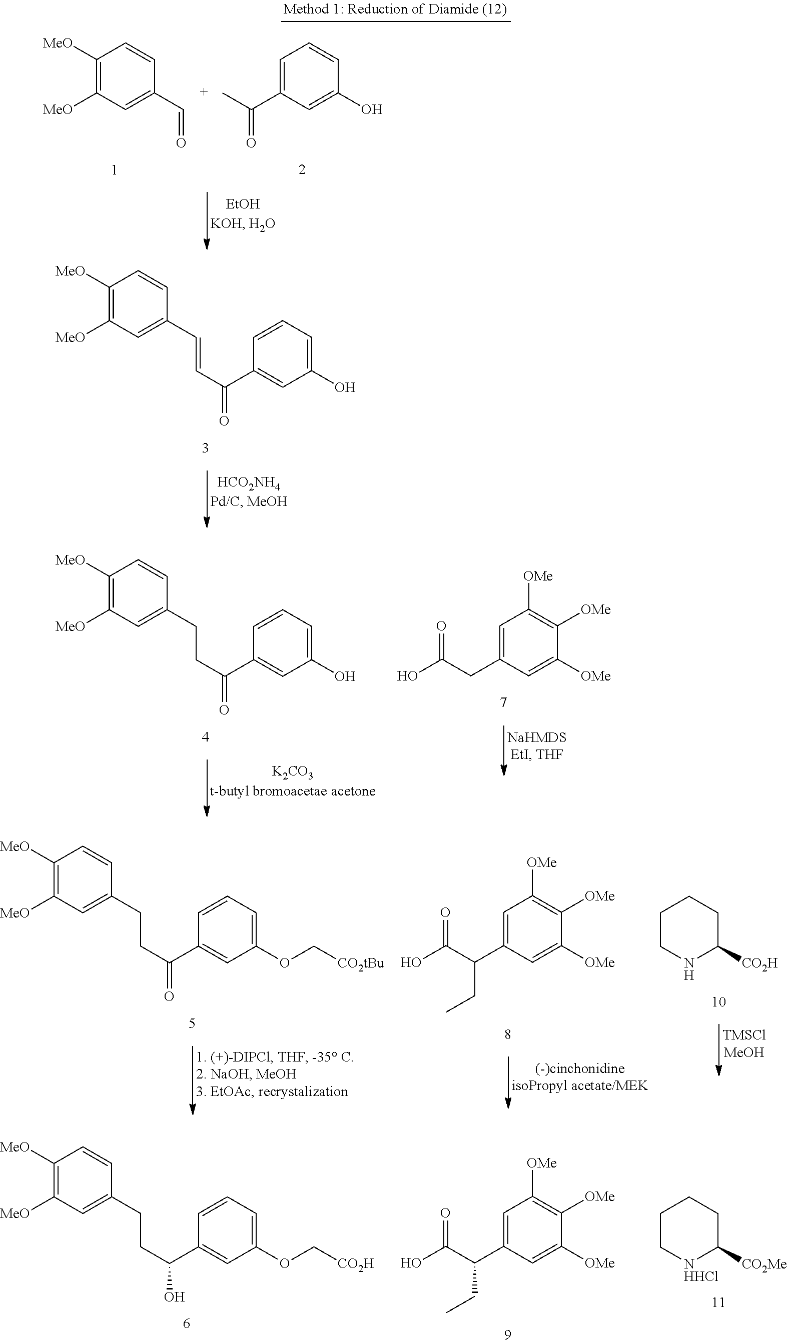

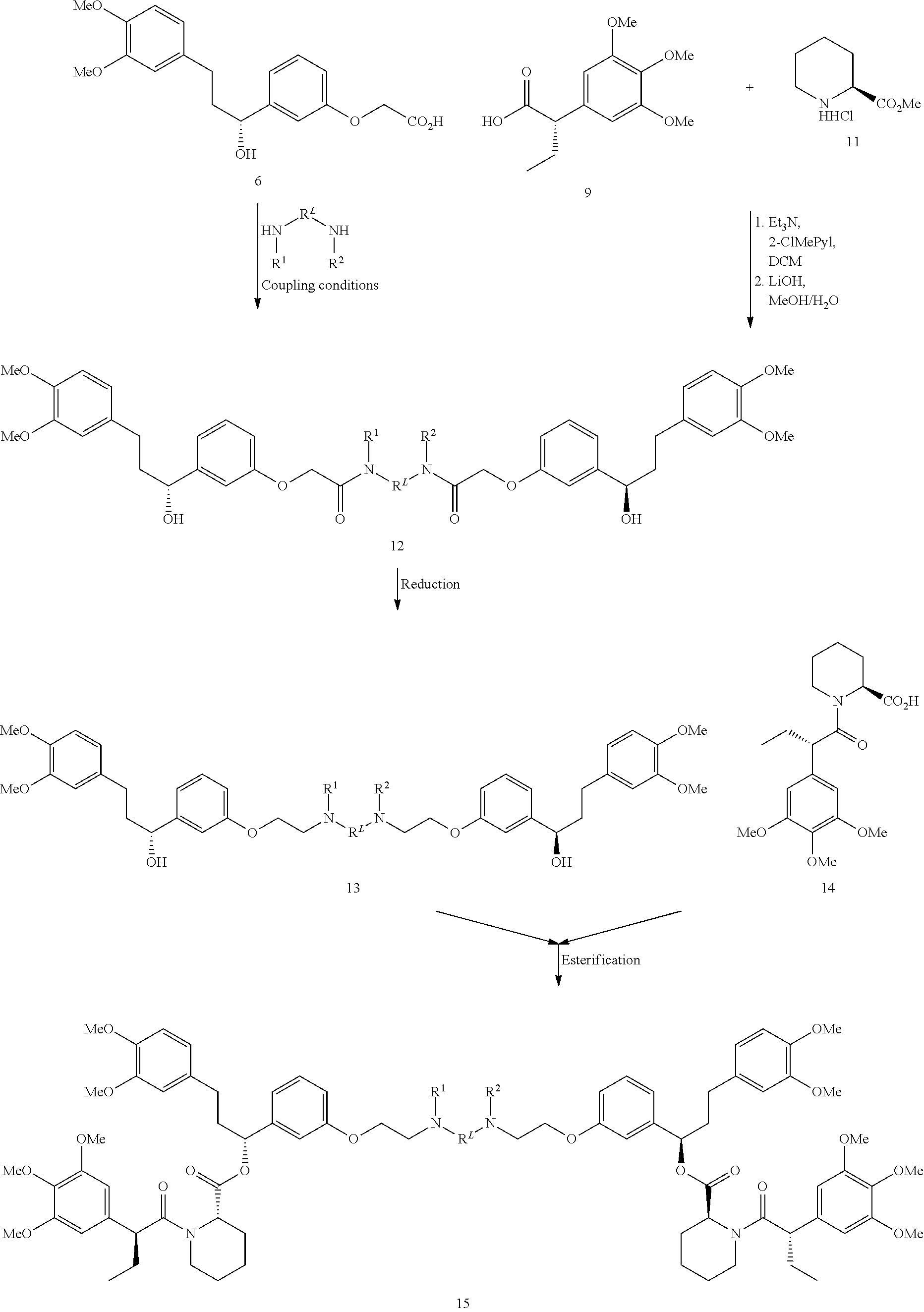

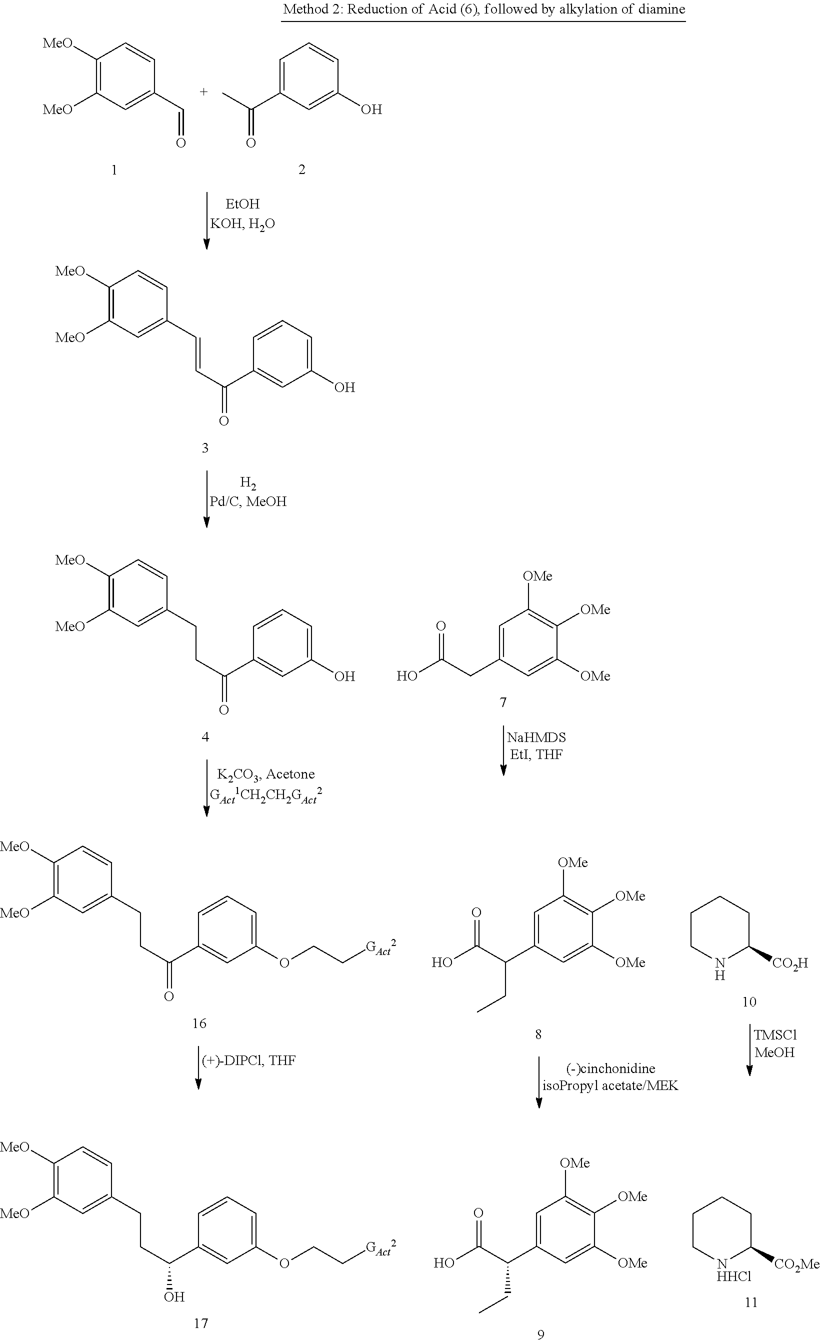

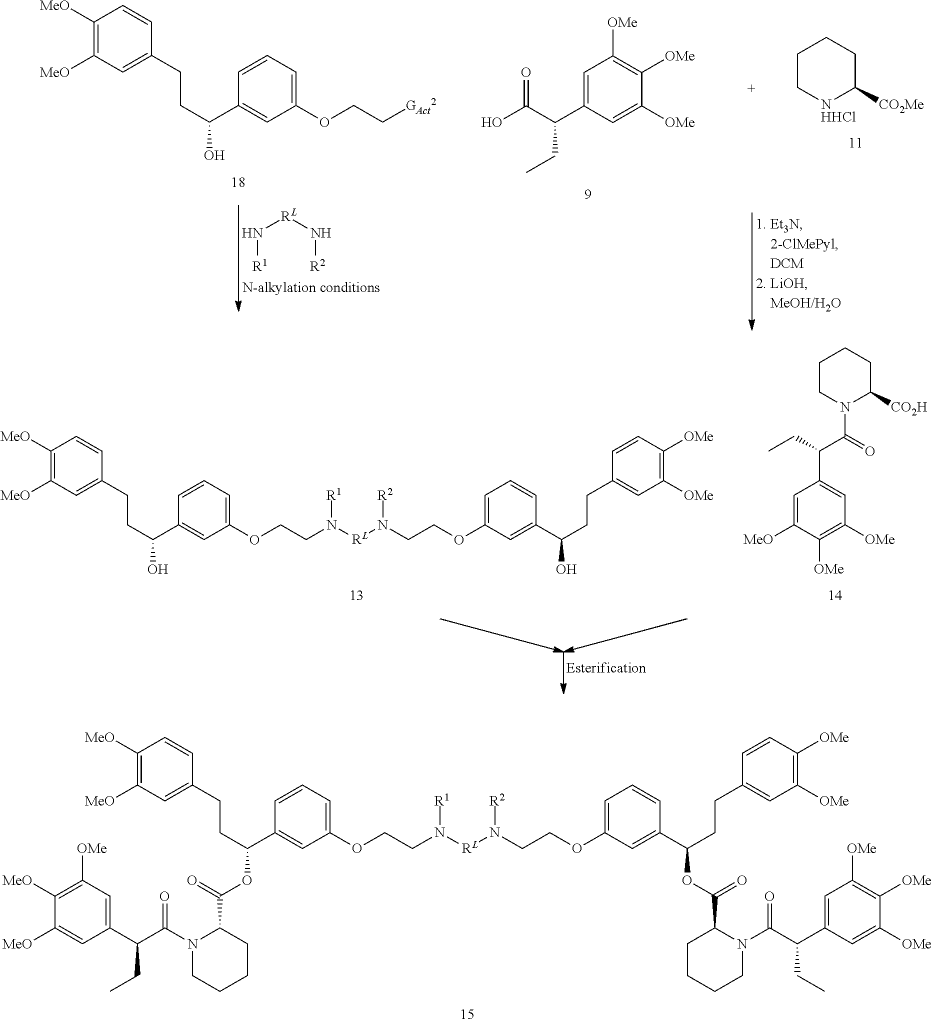

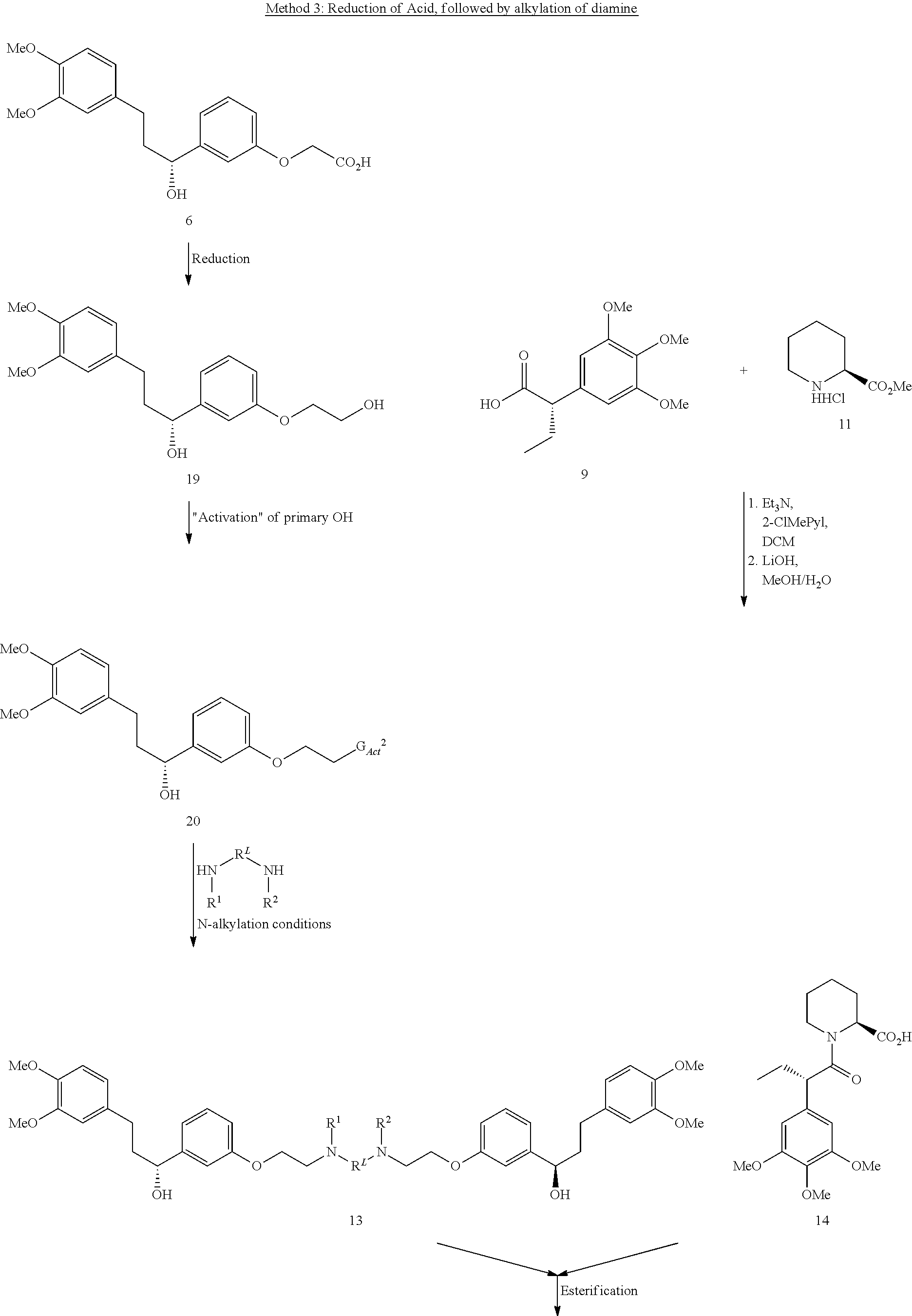

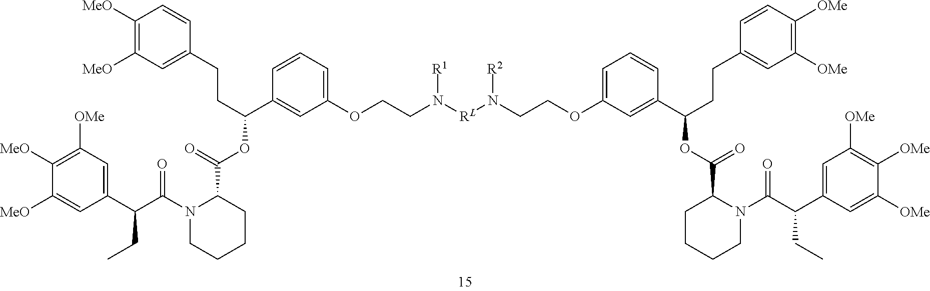

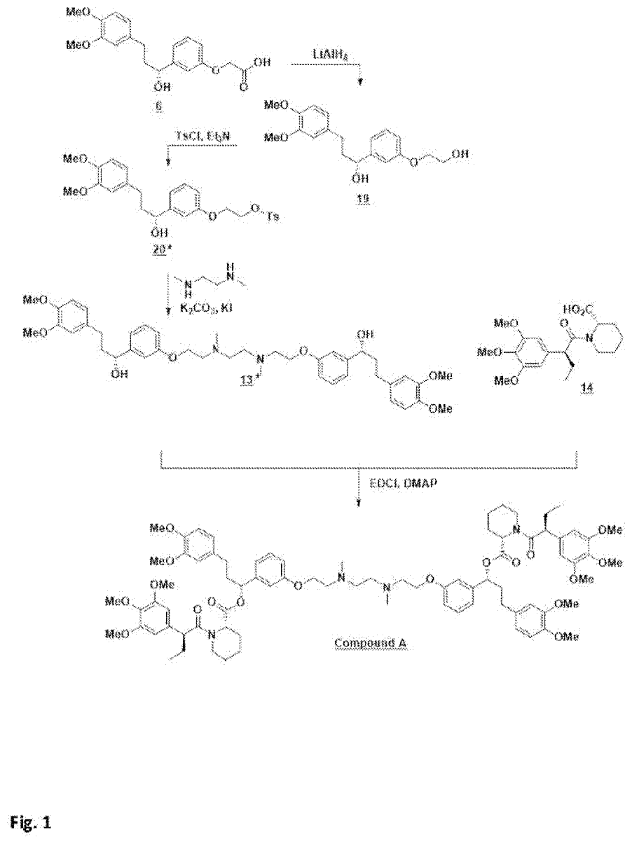

Method 3 is a modification of Method 2, and is the method similar to that used for the synthesis of Compound A according to FIG. 1.

GAct¹ and GAct² are groups that may be used in the process of alkylation to activate the carbon atom to which the group is attached. The group can be removed at certain conditions, as required in the process, and subsequently substituted by, for example, but not limited to, NHR¹R^{L}NHR² or, for example, but not limited to, NR¹R^{L}NR². Non-limiting examples of GAct include halogen, OTs (O-tosyl), OMs, or OTf.

A compound of Formula I or II, where Y is M, may be modified to obtain a compound of Formula I or II, where Y is Q, via an N-alkylation reaction or a reaction with an acid. The reaction can be done with one or two equivalents of the acid or alkylating agent, to lead to a mono- or di-quaternary amine.

The term “Coupling conditions” as used herein refers to chemical reaction conditions used to create a chemical bond between N and CO, as in a peptide bond.

The term “reduction” as used herein refers to a chemical redox reaction in which the oxygen atom of a carboxilic group is substituted by two hydrogen atoms, reducing CO₂H to CH₂OH.

The term “Esterification” as used herein refers to chemical reaction conditions used to create an ester.

The term “Activation of primary OH” as used herein refers to a chemical process in which the primary alcohol is modified or substituted to create a labile group as used in an alkylation reaction.

A non-limiting example of modification is esterification, where a non-limiting example of an ester group is tosyl. A non-limiting example of a substitution reaction is a substitution of OH with a halogene.

The term “N-alkylation conditions’ as used herein refers to chemical reaction conditions used to form an N—R bond.

Example of a scheme for modifying a compound of Formula I or II, where Y is M, to obtain a compound of Formula I or II, where Y is Q:

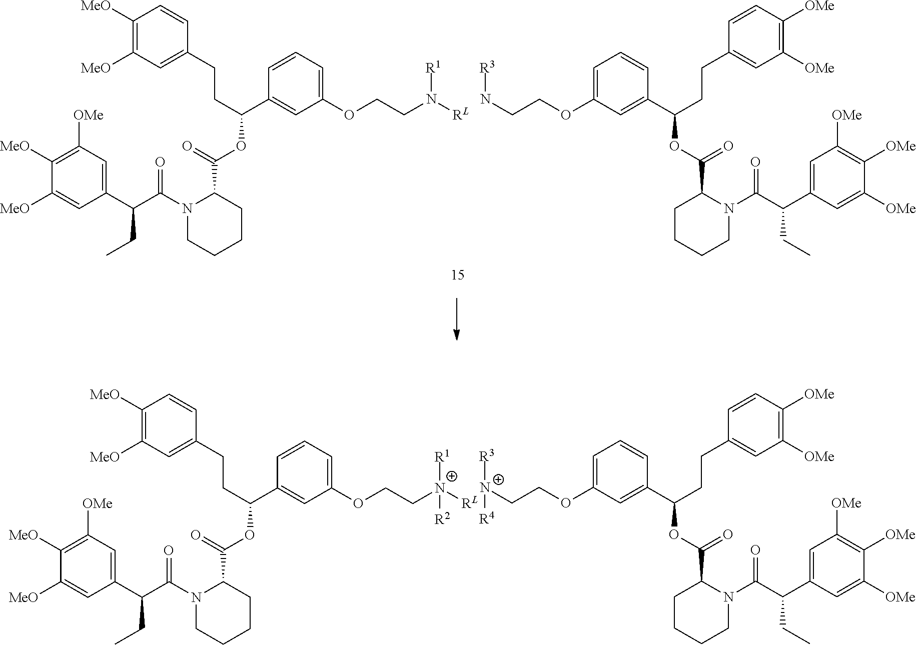

### Characterization of Multimeric Compounds

Multimeric compounds described herein, or pharmaceutically acceptable salts thereof, can be characterized for a particular property using a suitable method. Multimeric compounds described herein, or pharmaceutically acceptable salts thereof, can be characterized in a number of ways, including, for example, for binding characteristics to a protein and for solubility in water.

Binding Characteristics

Provided in some embodiments is a multimeric compound having a structure of Formula I or II, where the compound selectively binds to a FKBP polypeptide, or in certain embodiments, binds to a FKBP12 polypeptide or FKBP12 polypeptide variant. A FKBP12 polypeptide variant sometimes comprises an amino acid substitution at a position corresponding to position 36 in the wild type FKBP12 polypeptide. In some embodiments, the amino acid substitution is to an amino acid chosen from valine, leucine, isoleucine and alanine. In some embodiments, the amino acid substitution is to valine. In some embodiments, the FKBP12 polypeptide variant is FKBP12v36.

In some embodiments, a multimeric compound described herein, or a pharmaceutically acceptable salt thereof, binds to the FKBP12 polypeptide variant with an IC₅₀ at least 10 times lower than the IC₅₀ of the compound binding to the wild type FKBP12 polypeptide. In some embodiments, a multimeric compound described herein, or a pharmaceutically acceptable salt thereof, binds to the FKBP12 polypeptide variant with an IC₅₀ at least 100 times lower than the IC₅₀ of the compound binding to the wild type FKBP12 polypeptide. In some embodiments, a multimeric compound described herein, or a pharmaceutically acceptable salt thereof, binds to the FKBP12 polypeptide variant with an IC₅₀ at least 1000 times lower than the IC₅₀ of the compound binding to the wild type FKBP12 polypeptide. In some embodiments a multimeric compound described herein, or a pharmaceutically acceptable salt thereof, has a binding affinity (IC₅₀) to FKBP12v36 of 100 nM or less. In some embodiments, a multimeric compound provided herein, or a pharmaceutically acceptable salt thereof, binds to the FKBP12 polypeptide variant with an IC₅₀ at least 5, 10, 15, 20, 30, 40, 50, 60, 70, 80, 90, 100, 125, 150, 175, 200, 250, 300, 350, 400, 450, 500, 600, 700, 800, 900, 1000, 1250, 1500, 1750, 2000, 2250, 2500, 2750, 3000, 3500, 4000, 4500, or 5000, times lower than the IC₅₀ of the compound binding to the wild type FKBP12 polypeptide. In some embodiments, the FKBP12 polypeptide variant used to measure binding affinity has an amino acid substitution at amino acid residue or position 36. In some embodiments, the FKBP12 polypeptide variant used to measure binding affinity has a substitution at position 36 to an amino acid chosen from valine, leucine, isoleucine and alanine. In some embodiments, the FKBP12 polypeptide variant used to measure binding affinity has an amino acid substitution at position 36 to valine. In some embodiments, the FKBP12 polypeptide variant used to measure binding affinity is FKBP12v36.

In some embodiments methods are provided for multimerizing polypeptides expressed in a cell, comprising contacting the cell with a compound or a pharmaceutical composition provided herein, wherein the polypeptides comprise at least one FKBP12 polypeptide variant. In some embodiments, the at least one FKBP12 polypeptide variant comprises an amino acid substitution at a position corresponding to position 36 in the wild type FKBP12 polypeptide. In some embodiments, the amino acid substitution is to an amino acid chosen from valine, leucine, isoleucine and alanine. In some embodiments, the amino acid substitution is to valine. In some embodiments, the FKBP12 polypeptide variant is FKBP12v36. In some embodiments, the at least one FKBP12 polypeptide variant is Fv′Fvls.

Binding Affinity Assays

Compounds provided herein can be evaluated for the ability to bind proteins using methods described herein or known in the art. For example, compounds provided herein can be evaluated for binding to one or more FKBP proteins (e.g., FKBP12) and/or variants of FKBP proteins. By being “capable of binding”, as in the example of a multimeric or heterodimeric ligand, or multimeric compound described herein, binding to a multimerizing region or ligand binding region is meant that the ligand binds to the ligand binding region, for example, a portion, or portions, of the ligand or multimeric compound bind to the multimerizing region, and that this binding may be detected by an assay method including, but not limited to, a biological assay, a chemical assay, or physical means of detection such as, for example, x-ray crystallography. In addition, where a ligand or multimeric compound is considered to “not significantly bind” is meant that there may be minor detection of binding of a ligand or multimeric compound to the ligand binding region, but that this amount of binding, or the stability of binding is not significantly detectable, and, when occurring in the cells of the present embodiment, does not activate the modified cell or cause apoptosis. In certain examples, where the ligand or multimeric compound does not “significantly bind,” upon administration of the ligand or multimeric compound, the amount of cells undergoing apoptosis is less than 10, 5, 4, 3, 2, or 1%.

The binding affinity of multimeric compounds described herein, or pharmaceutically acceptable salts thereof, may be determined by assaying binding to polypeptides, such as, for example, rimiducid-binding polypeptides. In some examples, the rimiducid-binding polypeptide is a multimerizing region polypeptide (multimeric ligand binding region), such as, for example, a FKBP12 polypeptide, or a FKBP12 polypeptide variant, such as, for example, FKBP12v36. Methods for measuring binding affinity may also include functional binding assays, such as, for example, measuring an activity associated with the multimerization of chimeric polypeptides expressed in cells, following treatment of the cells with the multimeric compounds described herein, or pharmaceutically acceptable salts thereof.

Some functional assays incorporate secreted alkaline phosphatase (SEAP) as a readily detectable reporter molecule. For example, cells may be transfected or transduced with nucleic acid encoding two separate inducible pro-apoptotic fusion proteins (each fusion containing one of two multimerizing region polypeptides being tested for binding), the cells are contacted with a compound described herein, or a pharmaceutically acceptable salt thereof, and multimerization-induced apoptosis is then measured using a SEAP assay. An example of a pro-apoptotic protein is caspase. Dose-response studies using such an assay can be used to determine binding affinity of a compound for a protein based on IC₅₀ values determined from the assay results. An example of a SEAP apoptosis-based assay that may be used to determine binding characteristics of a compound to a multimerization region is provided in Example 5.

### Solubility Characteristics

In some embodiments, multimeric compounds described herein, or pharmaceutically acceptable salts thereof, are soluble in water. In some embodiments, multimeric compounds described herein, or pharmaceutically acceptable salts thereof, are soluble in an acetate buffer having a pH of 6 or less. In some embodiments, multimeric compounds described herein, or pharmaceutically acceptable salts thereof, are soluble in an acetate buffer having a pH of 4 or less. In some embodiments, multimeric compounds described herein, or pharmaceutically acceptable salts thereof, are soluble in an acetate buffer having a pH of 7 or less, 6 or less, 5 or less, 4 or less, 3 or less, or 2 or less.

In some embodiments, multimeric compounds described herein, or pharmaceutically acceptable salts thereof, have a solubility in water greater than the solubility of rimiducid in water. In some embodiments, multimeric compounds described herein, or pharmaceutically acceptable salts thereof, are soluble at a concentration of 1 mg·mL⁻¹ or greater in water. In some embodiments, multimeric compounds described herein, or pharmaceutically acceptable salts thereof, are soluble at a concentration of 2.5 mg·mL⁻¹ or greater in water. In some embodiments, multimeric compounds described herein, or pharmaceutically acceptable salts thereof, are soluble at a concentration greater than 0.5, 1, 1.5. 2, 2.5, 3, 3.5, 4, 4.5, or 5 mg·mL⁻¹ in water.

In some embodiments, multimeric compounds described herein, or pharmaceutically acceptable salts thereof, have a solubility in an acetate buffer having a pH of 6 or less that is greater than the solubility of rimiducid in an acetate buffer having a pH of 6 or less. In some embodiments, multimeric compounds described herein, or pharmaceutically acceptable salts thereof, have a solubility in an acetate buffer having a pH of 5 or less that is greater than the solubility of rimiducid in an acetate buffer having a pH of 5 or less. In some embodiments, multimeric compounds described herein, or pharmaceutically acceptable salts thereof, have a solubility in an acetate buffer having a pH of 4 or less that is greater than the solubility of rimiducid in an acetate buffer having a pH of 4 or less. In some embodiments, multimeric compounds described herein, or pharmaceutically acceptable salts thereof, are soluble at a concentration greater than 4 mg·mL⁻¹ in an acetate buffer having a pH of 6 or less. In some embodiments, multimeric compounds described herein, or pharmaceutically acceptable salts thereof, are soluble at a concentration greater than 2, 2.5, 3, 3.5, 4, 4.5, 5, 5.5, 6, 6.5. 7, or 8 mg·mL⁻¹ in an acetate buffer having a pH of 6 or less.

In some embodiments, multimeric compounds described herein, or pharmaceutically acceptable salts thereof, are soluble at a concentration greater than 4 mg·mL⁻¹ in an acetate buffer having a pH of 5 or less. In some embodiments, multimeric compounds described herein, or pharmaceutically acceptable salts thereof, are soluble at a concentration greater than 2, 2.5, 3, 3.5, 4, 4.5, 5, 5.5, 6, 6.5. 7, or 8 mg·mL⁻¹ in an acetate buffer having a pH of 5 or less.

In some embodiments, multimeric compounds described herein, or pharmaceutically acceptable salts thereof, are soluble at a concentration greater than 0.2 mg·mL⁻¹ in an acetate buffer having a pH of 4 or less. In some embodiments, multimeric compounds described herein, or pharmaceutically acceptable salts thereof, are soluble at a concentration greater than 4 mg·mL⁻¹ in an acetate buffer having a pH of 4 or less. In some embodiments, multimeric compounds described herein, or pharmaceutically acceptable salts thereof, are soluble at a concentration greater than 0.2, 0.5, 0.75, 1, 1.25, 1.5, 1.75, 2, 2.5, 3, 3.5, 4, 4.5, 5, 5.5, 6, 6.5. 7, or 8 mg·mL⁻¹ in an acetate buffer having a pH of 4 or less.

In some embodiments, multimeric compounds described herein, or pharmaceutically acceptable salts thereof, are soluble in water. In some embodiments, multimeric compounds described herein, or pharmaceutically acceptable salts thereof, are soluble in a phosphate buffer having a pH of 6 or less. In some embodiments, multimeric compounds described herein, or pharmaceutically acceptable salts thereof, are soluble in a phosphate buffer having a pH of 4 or less. In some embodiments, multimeric compounds described herein, or pharmaceutically acceptable salts thereof, are soluble in a phosphate buffer having a pH of 7 or less, 6 or less, 5 or less, 4 or less, 3 or less, or 2 or less.

In some embodiments, multimeric compounds described herein, or pharmaceutically acceptable salts thereof, have a solubility in water greater than the solubility of rimiducid in water. In some embodiments, multimeric compounds described herein, or pharmaceutically acceptable salts thereof, are soluble at a concentration of 1 mg·mL⁻¹ or greater in water. In some embodiments, multimeric compounds described herein, or pharmaceutically acceptable salts thereof, are soluble at a concentration of 2.5 mg·mL⁻¹ or greater in water. In some embodiments, multimeric compounds described herein, or pharmaceutically acceptable salts thereof, are soluble at a concentration greater than 0.5, 1, 1.5. 2, 2.5, 3, 3.5, 4, 4.5, or 5 mg·mL⁻¹ in water.

In some embodiments, multimeric compounds described herein, or pharmaceutically acceptable salts thereof, have a solubility in a phosphate buffer having a pH of 6 or less that is greater than the solubility of rimiducid in n phosphate buffer having a pH of 6 or less. In some embodiments, multimeric compounds described herein, or pharmaceutically acceptable salts thereof, have a solubility in a phosphate buffer having a pH of 5 or less that is greater than the solubility of rimiducid in a phosphate buffer having a pH of 5 or less. In some embodiments, multimeric compounds described herein, or pharmaceutically acceptable salts thereof, have a solubility in a phosphate buffer having a pH of 4 or less that is greater than the solubility of rimiducid in a phosphate buffer having a pH of 4 or less. In some embodiments, multimeric compounds described herein, or pharmaceutically acceptable salts thereof, are soluble at a concentration greater than 4 mg·mL⁻¹ in a phosphate buffer having a pH of 6 or less. In some embodiments, multimeric compounds described herein, or pharmaceutically acceptable salts thereof, are soluble at a concentration greater than 2, 2.5, 3, 3.5, 4, 4.5, 5, 5.5, 6, 6.5. 7, or 8 mg·mL⁻¹ in a phosphate buffer having a pH of 6 or less.

In some embodiments, multimeric compounds described herein, or pharmaceutically acceptable salts thereof, are soluble at a concentration greater than 4 mg·mL⁻¹ in a phosphate buffer having a pH of 5 or less. In some embodiments, multimeric compounds described herein, or pharmaceutically acceptable salts thereof, are soluble at a concentration greater than 2, 2.5, 3, 3.5, 4, 4.5, 5, 5.5, 6, 6.5. 7, or 8 mg·mL⁻¹ in a phosphate buffer having a pH of 5 or less.

In some embodiments, multimeric compounds described herein, or pharmaceutically acceptable salts thereof, are soluble at a concentration greater than 0.2 mg·mL⁻¹ in a phosphate buffer having a pH of 4 or less. In some embodiments, multimeric compounds described herein, or pharmaceutically acceptable salts thereof, are soluble at a concentration greater than 4 mg·mL⁻¹ in a phosphate buffer having a pH of 4 or less. In some embodiments, multimeric compounds described herein, or pharmaceutically acceptable salts thereof, are soluble at a concentration greater than 0.2, 0.5, 0.75, 1, 1.25, 1.5, 1.75, 2, 2.5, 3, 3.5, 4, 4.5, 5, 5.5, 6, 6.5. 7, or 8 mg·mL⁻¹ in a phosphate buffer having a pH of 4 or less.

By “soluble” or “solubility” is meant the property of a multimeric compound to dissolve in water, buffer, or other liquid, and may be measured in terms of mg·mL⁻¹. Solubility may be assessed with reference to water, or a buffered solution such as, for example, a solution buffered by acetate, phosphate, citrate or other buffering agent suitable for a buffer solution having a pH of 7 or less, 6 or less, 5 or less, or 4 or less. Examples of pharmaceutically acceptable buffers include those provided in Remington: The Science and Practice of Pharmacy, 22 ed., 2013, Pharmaceutical Press, London (Allen, L. V., Jr., ed.), which is hereby incorporated by reference herein in its entirety for all purposes. Where the buffer or pH of the liquid is not provided herein, such as when, for example, the solubility of a multimeric compound is discussed alone, or by comparison to a control compound such as rimiducid, the reference liquid is water.

Evaluating Solubility of Compounds

By “soluble” or “solubility” is meant the property of a compound to dissolve in water, buffer, or other liquid, which may be measured in terms of mg·mL⁻¹. Solubility may be assessed with reference to water, or a buffered solution such as, for example, a solution buffered by acetate, phosphate, citrate or other buffering agent suitable for a buffer solution having a pH of 7 or less, 6 or less, 5 or less, or 4 or less. Examples of pharmaceutically acceptable buffers include those provided in Remington: The Science and Practice of Pharmacy, 22 ed., 2013, Pharmaceutical Press, London (Allen, L. V., Jr., ed.). Where the buffer or pH of the liquid is not provided herein, such as when, for example, the solubility of a compound is discussed alone, or by comparison to a control compound such as rimiducid, the reference liquid is, for example, water. Methods for evaluating the solubility of a compound include, for example, a 96-well plate-based assay in which aqueous suspensions of the compound are vacuum-filtered and the concentration of compound is measured by UV spectrophotometry (Roy et al (2001) Drug Dev Ind Pharm 27(1):107-109).

### Evaluating Immunosuppressive Activity of Compounds

Compounds provided herein can be evaluated for immunosuppressive effects using methods known in the art. For example, in vitro methods for measuring immunosuppressive activity include activated lymphocyte or splenocyte proliferation assays (see, e.g., Collinge et al. (2010) J Immunotoxicol 7:357-366; Luengo et al. (1995) Chem Biol 2:471-481). An example of an in vivo method for measuring immunosuppressive activity is the animal model contact hypersensitivity assay (see, e.g., Olson et al. (2007) Int Immunopharmacol 7(6):734-743).

### Evaluating Antifungal Activity of Compounds

There are a number of in vitro assays for antifungal activity of compounds. For example, some assays are based on the inhibition of growth of Candida albicans (see, e.g., Clerya Alvino Leite et al. (2014) Evidence Based Complementary and Alternative Medicine doi.org/10.1155/2014/378280). Another example of a method for evaluating antifungal activity is an assay for inhibition of germ tube formation of Candida albicans (see, e.g., Brayman and Wlks (2003) Antimicrob Agents Chemother 47(10):3305-3310).

### Evaluating Antiproliferative Activity of Compounds

The antiproliferative activities of compounds can be evaluated using in vitro methods, for example in assays of tumor cell growth, and in vivo methods using animal xenograph models. In one method of determining the proliferation of tumor cells (e.g., human osteosarcoma cells) over time in the presence of a compound, the mitochondrial metabolic rate of cells is evaluated through detection of the absorbance of cells that have been seeded into 96-well plates, exposed to the compound and treated with MTT and MTS (tetrazolium compounds that are reduced by viable cells to generate a detectable formazan product) (see, e.g., Riss et al. 2013 Cell Viability Assays. In Assay Guidance Manual; Sittampalam et al., eds.; www.ncbi.nlm.nih.gov/books/NBK144065/). Antiproliferative activity of compounds in vivo can be evaluated, for example, using mouse xenograph models that can be generated through subcutaneous injection of tumor cells into mice. Tumor volumes of control and compound-treated mice can be compared to determine anti-tumor effects of a compound (see, e.g., Zhao et al (2015) JBUON 20(2):588-594).

### Evaluating Stability of Compounds

Some multimeric compounds described herein (e.g., solubility, binding characteristics) that are preferable for some uses of the analogs but may possess other properties, e.g., in vivo stability, that are diminished relative to rimiducid. For pharmaceutical uses, a compound should have suitable pharmacokinetic properties (e.g., good absorption, metabolic clearance rate and bioavailability).

Methods for evaluating the metabolic stability of a compound are described herein and/or known in the art. Assays using liver microsomes can be used as a rapid in vitro method to evaluate metabolic stability as a reasonably accurate prediction of in vivo, intrinsic hepatic clearance in a live whole organism (e.g., a mammal, such as a human). Because the liver is a major site of drug processing in the body, with a majority of drugs being metabolized through hepatic CYP-mediated mechanisms, liver microsomes contain membrane-bound metabolizing enzymes which makes them useful for in vitro assessment of metabolic stability of compounds. Examples of liver microsome-based assays for compound stability are described by Hill ((2003) Curr Protocols Pharmacol 7(8):1-7.8.11) and Knights et al. ((2016) Curr Protocols Pharmacol 74(1):7.8.1-7.8.24). In vivo metabolic stability assays of compounds can also be conducted in animal models using methods known in the art (see, e.g., Paoloni et al. [(2010) Rapamycin Pharmacokinetic and Pharmacodynamic Relationships in Osteosarcoma: A Comparative Oncology Study in Dogs. PLoS ONE 5(6): e11013. doi:10.1371/journal.pone.0011013] and Bouzas et al. [(2010) Upsala J Med Sci 115:125-130]).

In one example of the metabolic stability assay, liver microsomes can be prepared according to known methods and are commercially available (e.g., from XenoTech). Liver microsomes (0.5 mg/ml) in a reaction mixture containing potassium phosphate (pH 7.4; 100 mM), magnesium chloride (5 mM) and compound (1 pM) are equilibrated in a shaking water bath at 37° C. for 3 minutes. A control compound, such as, for example, testosterone, is run simultaneously with the test compound in a separate reaction. The reaction is initiated by the addition of NADPH cofactor (1 mM), and the mixture is incubated in a shaking water bath at 37° C. Aliquots (100 μl) are withdrawn at 0, 10, 20, 30 and 60 minutes. At each time point, withdrawn samples are immediately combined with ice-cold 50/50 acetonitrile (ACN)/H2O (400 μl) containing 0.1% formic acid and internal standard to terminate the reaction. The samples are then mixed and centrifuged to precipitate proteins. Samples are assayed by LC-MS/MS using electrospray ionization. The peak area response ratio (PARR) to internal standard is compared to the PARR at time 0 to determine the percent remaining at each time point. Half-lives are calculated using GraphPad software, fitting to a single-phase exponential decay equation.

### Methods of Using the Compounds

Methods of Multimerizing Chimeric Polypeptides

Certain embodiments of some of the methods provided herein incorporate chemically induced dimerization (CID) for conditional control of one or more proteins. In addition to this technique being inducible, it also is reversible, due to degradation of a labile dimerizing agent or administration of a monomeric competitive inhibitor. In some embodiments, a CID system-based method provided herein uses a compound provided herein. Included in such embodiments, are compounds provided herein that have increased solubility in water, and/or buffers such as acetate and phosphate, that retain some or all of the bioactivity of rimiducid dimerize polypeptides genetically fused to the FK506-binding protein, FKBP12, or variants thereof, such as, for example, FKBP12 polypeptides having an amino acid substitution at position 36, such as, for example, FKBP12v36. Multimeric compounds described herein, or pharmaceutically acceptable salts thereof, bind to and multimerize polypeptides that contain multimeric ligand binding regions or multimerizing regions as discussed herein and can be used as the chemical inducer of multimerization in methods provided herein. The methods discussed herein for multimerizing chimeric polypeptides may also be used to assay multimeric compounds described herein, alone, or as compared to rimiducid.

The terms “chimeric,” “fusion” and “chimeric fusion” are used interchangeably herein with reference to a polypeptide containing two or more proteins (or a portion(s) of one or more of the two or more proteins) that have been joined to create a chimeric polypeptide. The two or more proteins (or portions thereof) may be directly joined to each other, wherein a terminal amino acid residue of one protein (or portion thereof) is directly bonded to a terminal amino acid residue of another protein (or portion thereof), or may be joined through one or more intervening elements (e.g., one or more amino acids that are not part of either protein, such as a linker or adapter, or a non-amino acid polymer). For example, a polypeptide that is produced from nucleic acid encoding a fusion of a multimerizing protein (or portion thereof) and another protein (e.g., a DNA-binding protein, transcription activation protein, pro-apoptotic protein or protein component of an immune cell activation pathway), or portion thereof, may be referred to as a chimeric, fusion or chimeric fusion polypeptide.

### Ligand-Controlled Cell Switches

In some embodiments, the multimeric compounds described herein, or pharmaceutically acceptable salts thereof, may be used to dimerize or multimerize chimeric polypeptides that each comprise one or more multimerizing regions. This dimerization or multimerization of the chimeric polypeptides expressed in a cell may switch protein function and alter cell physiology. In some embodiments, the multimeric compounds described herein, or pharmaceutically acceptable salts thereof, may be used as small molecule ligands for ligand-controlled apoptosis, or ligand-controlled cell activation.

Multimerizing Chimeric Polypeptides

In certain embodiments, certain methods provided herein incorporate chemically induced dimerization (CID) and produce a conditionally controlled protein or polypeptide. In addition to this technique being inducible, it also is reversible, due to the degradation of the labile dimerizing agent or administration of a monomeric competitive inhibitor. Multimeric compounds described herein, or pharmaceutically acceptable salts thereof, bind to and multimerize polypeptides that comprise the multimeric ligand binding regions or multimerizing regions discussed herein. The term “multimerize” refers to the dimerization of two polypeptides, or the multimerization of more than two polypeptides. The term “ligand binding region” is interchangeable with the terms “dimerizing” region or domain, “dimerization” region or domain, “multimerizing” polypeptide, region or domain, “dimeric ligand binding” polypeptide, region or domain, “multimerization” polypeptide, region or domain, and “multimeric ligand binding” polypeptide, region or domain.

A CID system generally is based upon the notion that aggregation of surface receptors and other cell surface proteins, or non-surface cytosolic proteins effectively activates downstream signaling cascades. A CID system typically makes use of a synthetic bivalent ligand to rapidly crosslink signaling molecules that are fused to ligand binding domains. This system has been used to trigger the oligomerization and activation of cell surface proteins (Spencer, D. M., et al., Science, 1993. 262: p. 1019-1024; Spencer D. M. et al., Curr Biol 1996, 6:839-847; Blau, C. A. et al., Proc Natl Acad. Sci. USA 1997, 94:3076-3081), or cytosolic proteins (Luo, Z. et al., Nature 1996, 383:181-185; MacCorkle, R. A. et al., Proc Natl Acad Sci USA 1998, 95:3655-3660), the recruitment of transcription factors to DNA elements to modulate transcription (Ho, S. N. et al., Nature 1996, 382:822-826; Rivera, V. M. et al., Nat. Med. 1996, 2:1028-1032) or the recruitment of signaling molecules to the plasma membrane to stimulate signaling (Spencer D. M. et al., Proc. Natl. Acad. Sci. USA 1995, 92:9805-9809; Holsinger, L. J. et al., Proc. Natl. Acad. Sci. USA 1995, 95:9810-9814). In addition, the synthetic ligands are resistant to protease degradation, making them more efficient at activating receptors in vivo than most delivered protein agents.

In the simplest embodiment, a CID system uses a dimeric analog of the lipid permeable immunosuppressant drug, FK506, which loses its normal bioactivity while gaining the ability to crosslink molecules genetically fused to the FK506-binding protein, FKBP12. In some examples, by fusing one or more FKBP12 polypeptides or polypeptide variants to a Caspase-9 polypeptide, one can stimulate Caspase-9 activity in a dimerizer drug-dependent, but ligand and ectodomain-independent manner. This provides the system with temporal control, reversibility using monomeric drug analogs, and enhanced specificity. The high affinity of third-generation AP20187/AP1903 CIDs for their binding domain, FKBP12, permits specific activation of the recombinant receptor in vivo without the induction of non-specific side effects through endogenous FKBP12. The amino acid at position 36 of wild type FKBP12 polypeptide is phenylalanine. FKBP12 polypeptide variants include, but are not limited to, those having amino acid substitutions at position 36, chosen from valine, leucine, isoleucine, and alanine. In some embodiments, FKBP12 polypeptide variants include, but are not limited to, those having amino acid substitutions at position 36, selected from the group consisting of valine, leucine, isoleucine, and alanine. FKBP12 variants having amino acid substitutions and deletions, such as FKBP12v36, that bind to a dimerizer drug, may also be used (Jemal, A. et al., CA Cancer J. Clinic. 58, 71-96 (2008); Scher, H. I. and Kelly, W. K., Journal of Clinical Oncology 11, 1566-72 (1993)). Examples of FKBP12 polypeptide variants, and of methods of using the CID system, include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology: From Small Molecules to Systems Biology and Drug Design (Schreiber, s., et al., eds., Wiley, 2007)).

A multimerizing region of expression constructs described herein often comprises a FKBP12 polypeptide, for example, a FKBP12 polypeptide variant. By FKBP12 polypeptide variant, or FKBP12 mutant, is meant a FKBP12 polypeptide that binds to a ligand, such as rimiducid or a multimeric compound described herein, or a pharmaceutically acceptable salt thereof, with at least 100 times more affinity than a wild type FKBP12 polypeptide, such as, for example, a wild type FKBP12 polypeptide comprising the amino acid sequence of SEQ ID NO: 2., or, for example, the wild type FKBP12 polypeptide consisting of the amino acid sequence of SEQ ID NO: 2.

In some embodiments, inducible chimeric polypeptides comprise an FᵥFᵥₗₛ sequence, which comprises two FKBP12v36 polypeptides F36V′-FKBP: F36V′-FKBP is a codon-wobbled version of F36V-FKBP. It encodes the identical polypeptide sequence as F36V-FKBP but has only 62% homology at the nucleotide level. F36V′-FKBP was designed to reduce recombination in retroviral vectors (Schellhammer, P. F. et al., J. Urol. 157, 1731-5 (1997)). F36V′-FKBP was constructed by a PCR assembly procedure. The transgene contains one copy of F36V′-FKBP linked directly to one copy of F36V-FKBP.

The transduced signal will normally result from ligand-mediated oligomerization of the chimeric protein molecules, i.e., as a result of oligomerization following ligand binding, although other binding events, for example allosteric activation, can be employed to initiate a signal. The construct of the chimeric protein will vary as to the order of the various domains and the number of repeats of an individual domain.

Ligand-Controlled Cell Switch Methods

Ligand-Controlled Apoptosis

In some embodiments, a chimeric polypeptide is provided, or a nucleic acid encoding such a polypeptide is provided, or a cell that contains such a polypeptide or nucleic acid, for the purpose of inducing cell death in response to a multimeric compound described herein, or a pharmaceutically acceptable salt thereof. In certain embodiments, a chimeric polypeptide comprises one or more ligand binding regions, or multimerizing regions and an apoptosis-inducing polypeptide, such as, for example, caspase polypeptide, for example, a modified Caspase-9 polypeptide that lacks the CARD domain. Contacting the multimerizing region, for example, by contacting a cell that expresses the chimeric polypeptide, with a multimeric compound described herein, or a pharmaceutically acceptable salt thereof leads to multimerization of two or more chimeric caspase polypeptides, which results in apoptosis. Ligand-controlled apoptosis may also be used as an assay to determine the binding of the multimeric compounds described herein, or pharmaceutically acceptable salts thereof.

As used herein, the term “iCaspase-9” molecule, polypeptide, or protein is defined as an inducible Caspase-9. The term “iCaspase-9” embraces iCaspase-9 nucleic acids, iCaspase-9 polypeptides and/or iCaspase-9 expression vectors. The term also encompasses either the natural iCaspase-9 nucleotide or amino acid sequence, or a truncated sequence that is lacking the CARD domain. By “wild type” Caspase-9 in the context of the experimental details provided herein, is meant the Caspase-9 molecule lacking the CARD domain.

As used herein, the term “iCaspase 1 molecule”, “iCaspase 3 molecule”, or “iCaspase 8 molecule” is defined as an inducible Caspase 1, 3, or 8, respectively. The term iCaspase 1, iCaspase 3, or iCaspase 8, embraces iCaspase 1, 3, or 8 nucleic acids, iCaspase 1, 3, or 8 polypeptides and/or iCaspase 1, 3, or 8 expression vectors, respectively. The term also encompasses either the natural CaspaseiCaspase-1, -3, or -8 nucleotide or amino acid sequence, respectively, or a truncated sequence that is lacking the CARD domain.

Modified Caspase-9 polypeptides comprise at least one amino acid substitution that affects basal activity or IC50, in a chimeric polypeptide comprising the modified Caspase-9 polypeptide. Methods for testing basal activity and IC50 are discussed herein. Non-modified Caspase-9 polypeptides do not comprise this type of amino acid substitution. Both modified and non-modified Caspase-9 polypeptides may be truncated, for example, to remove the CARD domain.

In some embodiments, where the expression construct encodes a truncated Caspase-9 polypeptide, the truncated Caspase-9 polypeptide is encoded by the nucleotide sequence of SEQ ID NO 5, or a functionally equivalent fragment thereof, with or without DNA linkers, or has the amino acid sequence of SEQ ID NO: 6, or a functionally equivalent fragment thereof. In some embodiments, the CD19 polypeptide is encoded by the nucleotide sequence of SEQ ID NO 9, or a functionally equivalent fragment thereof, with or without DNA linkers, or has the amino acid sequence of SEQ ID NO: 10, or a functionally equivalent fragment thereof. A functionally equivalent fragment of the Caspase-9 polypeptide has substantially the same ability to induce apoptosis as the polypeptide of SEQ ID NO: 6, with at least 50%, 60%, 70%, 80%, 90%, or 95% of the activity of the polypeptide of SEQ ID NO: 6. In some embodiments, the expression construct encodes a truncated Caspase-9 polypeptide encoded by the Caspase-9 nucleotide sequence of pM101-pSFG-iC9.T2A-ΔCD19. In some embodiments, the Caspase-9 polypeptide comprises the amino acid sequence of the Caspase-9 polypeptide encoded by pM101-pSFG-iC9.T2A-ΔCD19.

“Function-conservative variants” of Caspase-9, or other proteins discussed herein, are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

As used herein, the term “functionally equivalent,” as it relates to Caspase-9, or truncated Caspase-9, for example, refers to a Caspase-9 nucleic acid fragment, variant, or analog, refers to a nucleic acid that codes for a Caspase-9 polypeptide, or a Caspase-9 polypeptide, that stimulates an apoptotic response. “Functionally equivalent” refers, for example, to a Caspase-9 polypeptide that is lacking the CARD domain, but is capable of inducing an apoptotic cell response. When the term “functionally equivalent” is applied to other nucleic acids or polypeptides, such as, for example, CD19, the 5′LTR, the multimeric ligand binding region, multimerizing region, or CD3, it refers to fragments, variants, and the like that have the same or similar activity as the reference polypeptides of the methods herein.

Non-limiting examples of chimeric polypeptides useful for inducing cell death or apoptosis, and related methods for inducing cell death or apoptosis, including expression constructs, methods for constructing vectors, assays for activity or function of the multimeric compounds described herein, multimerization of the chimeric polypeptides by contacting cells that express inducible chimeric polypeptides with a multimeric compound described herein, or a pharmaceutically acceptable salt thereof, that binds to the multimerizing region of the chimeric polypeptides both ex vivo and in vivo, administration of expression vectors, cells, or multimeric compounds described herein, or pharmaceutically acceptable salts thereof, to subjects, and administration of multimeric compounds described herein, or pharmaceutically acceptable salts thereof, to subjects who have been administered cells that express the inducible chimeric polypeptides, may also be found in the following patents and patent applications, each of which is incorporated by reference herein in its entirety for all purposes. U.S. patent application Ser. No. 13/112,739, filed May 20, 2011, entitled METHODS FOR INDUCING SELECTIVE APOPTOSIS, published Nov. 24, 2011, as US2011-0286980-A1, issued Jul. 28, 2015 as U.S. Pat. No. 9,089,520; U.S. patent application Ser. No. 13/792,135, filed Mar. 10, 2013, entitled MODIFIED CASPASE POLYPEPTIDES AND USES THEREOF, published Sep. 11, 2014 as US2014-0255360-A1, issued Sep. 6, 2016 as U.S. Pat. No. 9,434,935, by Spencer et al.; International Patent Application No. PCT/US2014/022004, filed Mar. 7, 2014, published Oct. 9, 2014 as WO2014/16438; U.S. patent application Ser. No. 14/296,404, filed Jun. 4, 2014, entitled METHODS FOR INDUCING PARTIAL APOPTOSIS USING CASPASE POLYPEPTIDES, published Jun. 2, 2016 as US2016-0151465-A1, by Slawin et al; International Application No. PCT/US2014/040964 filed Jun. 4, 2014, published as WO2014/197638 on Feb. 5, 2015, by Slawin et al.; U.S. patent application Ser. No. 14/640,553, filed Mar. 6, 2015, entitled CASPASE POLYPEPTIDES HAVING MODIFIED ACTIVITY AND USES THEREOF, published Nov. 19, 2015 as US2015-0328292-A1; International Patent Application No. PCT/US2015/019186, filed Mar. 6, 2015, published Sep. 11, 2015 as WO2015/134877, by Spencer et al.; U.S. patent application Ser. No. 14/968,737, filed Dec. 14, 2015, entitled METHODS FOR CONTROLLED ELIMINATION OF THERAPEUTIC CELLS, published Jun. 16, 2016 as US2016-0166613-A1, by Spencer et al.; International Patent Application No. PCT/US2015/065629 filed Dec. 14, 2015, published Jun. 23, 2016 as WO2016/100236, by Spencer et al.; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; and International Patent Application No. PCT/US2016/066371, filed Dec. 13, 2016, published Jun. 22, 2017 as WO2017/106185, by Bayle et al., each of which is incorporated by reference herein in its entirety for all purposes. Multimeric compounds described herein, or pharmaceutically acceptable salts thereof, may be used essentially as discussed in examples provided in these publications, and other examples provided herein, to the extent that they refer to multimeric ligands, such as, for example, rimiducid (AP1903) or AP20187.

Ligand-Controlled Cell Activation

In some embodiments, a chimeric polypeptide is provided, or a nucleic acid encoding such a polypeptide is provided, or a cell that contains such a polypeptide or nucleic acid is provided, for the purpose of inducing cell activation in response to a multimeric compound described herein, or a pharmaceutically acceptable salt thereof. For example, costimulating polypeptides may be used to enhance the activation of T cells, and of CAR-expressing T cells against target antigens, which would increase the potency of adoptive immunotherapy.

In some embodiments, the chimeric polypeptide comprises one or more ligand binding regions, or multimerizing regions, and a costimulating polypeptide. Contacting the multimerizing region, for example, by contacting a cell that express the chimeric polypeptide, with a multimeric compound described herein, or a pharmaceutically acceptable salt thereof, leads to multimerization of two or more chimeric polypeptides, which leads to activation of the cell. In some embodiments, the cell is a T cell. In some embodiments, the cell co-expresses a chimeric antigen receptor or a recombinant T cell receptor that recognizes an antigen expressed by a target cell. Contacting cells that express the chimeric costimulating polypeptide with the multimeric compound described herein, or a pharmaceutically acceptable salt thereof results in multimerization of the chimeric polypeptides, and activation of the immune activity of the cell, which, in some embodiments, leads to increased elimination of target cells.

Costimulating polypeptides provided in the chimeric polypeptides herein are capable of amplifying the cell-mediated immune response through activation of signaling pathways involved in cell survival and proliferation. Costimulating polypeptides may include any molecule or polypeptide that activates the NF-kappaB pathway, Akt pathway, and/or p38 pathway. Non-limiting examples of costimulating polypeptides include, for example, but are not limited, to the members of tumor necrosis factor receptor (TNFR) family (i.e., CD40, RANK/TRANCE-R, OX40, 4-1BB) and CD28 family members (CD28, ICOS), and may also include pattern recognition receptor adapters such as, for example MyD88. Chimeric polypeptides may comprise one, two, three, or more costimulating polypeptides.

Non-limiting examples of chimeric polypeptides useful for inducing cell activation, and related methods for inducing cell activation using a multimeric compound described herein, including expression constructs, methods for constructing vectors, assays for activity or function of the multimeric compounds described herein, multimerization of the chimeric polypeptides by contacting cells that express inducible chimeric polypeptides with a multimeric compound described herein, or a pharmaceutically acceptable salt thereof, that binds to the multimerizing region of the chimeric polypeptides both ex vivo and in vivo, administration of expression vectors, cells, or multimeric compounds described herein, or pharmaceutically acceptable salts thereof, to subjects, and administration of multimeric compounds described herein, or pharmaceutically acceptable salts thereof, to subjects who have been administered cells that express the inducible chimeric polypeptides, may also be found in the following patents and patent applications, each of which is incorporated by reference herein in its entirety for all purposes. U.S. patent application Ser. No. 14/210,034, filed Mar. 13, 2014, entitled METHODS FOR CONTROLLING T CELL PROLIFERATION, published Sep. 25, 2014 as US2014-0286987-A1; International Patent Application No. PCT/US2014/026734, filed Mar. 13, 2014, published Sep. 25, 2014 as WO2014/151960, by Spencer et al.; U.S. patent application Ser. No. 14/622,018, filed Feb. 13, 2014, entitled METHODS FOR ACTIVATING T CELLS USING AN INDUCIBLE CHIMERIC POLYPEPTIDE, published Feb. 18, 2016 as US2016-0046700-A1; International Patent Application No. PCT/US2015/015829, filed Feb. 13, 2015, published Aug. 20, 2015 as WO2015/123527; U.S. patent application Ser. No. 10/781,384, filed Feb. 18, 2004, entitled INDUCED ACTIVATION OF DENDRITIC CELLS, published Oct. 21, 2004 as US2004-0209836-A1, issued Jun. 29, 2008 as U.S. Pat. No. 7,404,950, by Spencer et al.; International Patent Application No. PCT/US2004/004757, filed Feb. 18, 2004, published Mar. 24, 2005 as WO2004/073641A3; U.S. patent application Ser. No. 12/445,939, filed Oct. 26, 2010, entitled METHODS AND COMPOSITIONS FOR GENERATING AN IMMUNE RESPONSE BY INDUCING CD40 AND PATTERN RECOGNITION RECEPTORS AND ADAPTORS THEREOF, published Feb. 10, 2011 as US2011-0033388-A1, issued Apr. 8, 2014 as U.S. Pat. No. 8,691,210, by Spencer et al.; International Patent Application No. PCT/US2007/081963, filed Oct. 19, 2007, published Apr. 24, 2008 as WO2008/049113; U.S. patent application Ser. No. 13/763,591, filed Feb. 8, 2013, entitled METHODS AND COMPOSITIONS FOR GENERATING AN IMMUNE RESPONSE BY INDUCING CD40 AND PATTERN RECOGNITION RECEPTOR ADAPTERS, published Mar. 27, 2014 as US2014-0087468-A1, issued Apr. 19, 2016 as U.S. Pat. No. 9,315,559, by Spencer et al.; International Patent Application No. PCT/US2009/057738, filed Sep. 21, 2009, published Mar. 25, 2010 as WO201033949; U.S. patent application Ser. No. 13/087,329, filed Apr. 14, 2011, entitled METHODS FOR TREATING SOLID TUMORS, published Nov. 24, 2011 as US2011-0287038-A1, by Slawin et al.; International Patent Application No. PCT/US2011/032572, filed Apr. 14, 2011, published Oct. 20, 2011 as WO2011/130566, by Slawin et al; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/047957, published as WO2016/036746 on Mar. 10, 2016, entitled COSTIMULATION OF CHIMERIC ANTIGEN RECEPTORS BY MYD88 AND CD40 POLYPEPTIDES; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1, by Bayle et al.; International Patent Application No. PCT/US2016/066371, filed Dec. 13, 2016, published Jun. 22, 2017 as WO2017/106185, by Bayle et al.; International Patent Application No. PCT/US2018/031689, filed May 8, 2018, entitled METHODS TO AUGMENT OR ALTER SIGNAL TRANSDUCTION, published Nov. 15, 2018 as WO2018/208849, by Bayle et al., each of which is incorporated by reference herein in its entirety, including all text, tables and drawings, for all purposes. Multimeric compounds described herein, or pharmaceutically acceptable salts thereof, may be used essentially as discussed in examples provided in these publications, and other examples provided herein, to the extent that they refer to multimeric ligands, such as, for example, rimiducid (AP1903) or AP20187.

### Methods of Cell Surface Protein Multimerization

Provided herein are methods of multimerizing cell surface proteins, e.g., cell surface receptors. The methods include a step of contacting cells expressing fusion proteins containing cell surface proteins, or portions thereof, and a ligand-binding domain that binds to a compound provided herein, or pharmaceutically acceptable salts thereof. In one example of these methods, a cell is transfected or transduced with (1) nucleic acid encoding a fusion of one cell surface protein, or portion thereof, and a FKBP12 protein (or variant thereof) and (2) nucleic acid encoding a fusion of a second cell surface protein (e.g., the same as or different from the cell surface protein fused to a FKBP12 protein, or variant thereof), or portion thereof. The cell is then contacted with a compound provided herein, or pharmaceutically acceptable salts thereof, that binds to and multimerizes the FKBP12 or FKBP12 variant proteins contained in the fusion proteins (e.g., heterodimers) and may be monitored for particular activities, such as, for example, changes in cell structure, function, protein phosphorylation (e.g., cell surface protein phosphorylation), receptor internalization or cell signaling. Such methods are useful, for example, in dissecting cell signaling pathways and elucidating protein associations within cells. Methods of generating nucleic acid vectors for expression of fusion proteins, transfecting and transducing cells with the nucleic acids, and monitoring cells for multimerization of fusion proteins and effects thereof are described herein and/or known to those of skill in the art (see, e.g., Song and Hinkle (2005) Mol Endocrinol 19(11):2859-2870; Spencer et al. (1993) Science 262:1019-1024; Spencer et al. (1996) Curr Biol 6:839-847; Blau et al., (1997) Proc Natl Acad Sci USA 94:3076-3081; Muthuswamy et al. (1999) Mol Cell Biol 19(10):6845-6857; Otto et al. (2001) Blood 97:3662-3664).

### Methods of Protein Translocation and Recruitment to Cellular Membranes

Provided herein are methods of intracellular protein translocation and protein recruitment to cellular membranes through multimerization induced by compounds provided herein, or pharmaceutically acceptable salts thereof. In one example of these methods, a cell is transfected or transduced with (1) nucleic acid encoding a fusion of an intracellular protein, or portion thereof, and a FKBP12 protein (or variant thereof) and (2) nucleic acid encoding a fusion of a plasma membrane-targeting myristoylation signal protein and a FKBP12 protein (or variant thereof). The cell is then contacted with a compound provided herein, or pharmaceutically acceptable salts thereof, that binds to and multimerizes the FKBP12 (or variant) proteins contained in the fusion proteins and may be monitored for localization of the intracellular protein fusion to the plasma membrane and/or particular activities, such as, for example, cell signal transduction, associated with plasma membrane localization. Such methods are useful, for example, in dissecting cell signaling pathways and protein localization requirements thereof as well as in inducing a reaction at the plasma membrane or other membrane. Methods of generating nucleic acid vectors for expression of fusion proteins, transfecting and transducing cells with the nucleic acids, and monitoring cells for multimerization of fusion proteins and effects (e.g., protein translocation) thereof are described herein and/or known to those of skill in the art (see, e.g., Putyrski and Schultz (2012) FEBS Lett 586(15):2091-2105; van Unen et al (2016) Nature Scientific Reports vol. 6, article number 36825, https://doi.org/10.1038/srep36825).

In an example of another method provided herein, a cell is transfected or transduced with (1) nucleic acid encoding a fusion of a nuclear localization signal (NLS)/DNA-binding protein (e.g., GAL4), or portion thereof, and a FKBP12 protein (or variant thereof) and (2) nucleic acid encoding a fusion of a nuclear export signal (NES) protein and a FKBP12 protein (or variant thereof). The cell is then contacted with a compound provided herein, or pharmaceutically acceptable salts thereof, that binds to and multimerizes the FKBP12 proteins contained in the fusion proteins and may be monitored for localization of the nucleus-targeted protein fusion to the cytoplasm. Such methods are useful in, for example, the identification of nuclear export signals and inducibly shuttling proteins between the nucleus and the cytoplasm. Methods of generating nucleic acid vectors for expression of fusion proteins, transfecting and transducing cells with the nucleic acids, and monitoring cells for multimerization of fusion proteins and effects (e.g., protein translocation) thereof are described herein and/or known to those of skill in the art (see, e.g., Terrillon and Bouvier (2004) EMBO J 23:3950-3961; Heo et al. (2006) Science 314(5804):1458-1461; Klemm et al. (1997) Curr Biol 7:638-644; Bayle et al. (2006) Chem Biol 13:99-107).

### Methods of Recruiting Nucleic Acid-Binding Proteins to Nucleic Acids

Provided herein are methods of recruiting nucleic acid-binding proteins to nucleic acids through multimerization induced by compounds provided herein, or pharmaceutically acceptable salts thereof. Examples of nucleic acid-binding proteins include, but are not limited to, transcription factors and splicing regulator proteins (e.g., SR proteins or RS domains thereof; see, e.g., Graveley (2005) RNA 11:355-358). In one example of these methods, a cell is transfected or transduced with (1) nucleic acid encoding a fusion of a DNA binding-domain (including a nuclear localization signal) protein, or portion thereof, and a FKBP12 protein (or variant thereof) or portion thereof and (2) nucleic acid encoding a fusion of a transcription factor activation domain (including a nuclear localization signal) protein and a FKBP12 protein (or variant thereof) or portion thereof. If dimerization-induced transcription in the cell is intended to be from a heterologous DNA vector, the cell is also transfected or transduced with that vector which includes DNA to which the DNA-binding domain of one of the fusion proteins binds. The cell is then contacted with a compound provided herein, or pharmaceutically acceptable salts thereof, that binds to and multimerizes the FKBP12 (or variant thereof) proteins contained in the fusion proteins and may be monitored for transcription of an endogenous or heterologous gene. This method is useful, for example, in pharmacologic control of gene expression in gene therapy and in generating reporter gene transcription-based assays in cells to identify multimerizing ligand-binding proteins that bind to the compound being administered to the cells. Methods of generating nucleic acid vectors for expression of fusion proteins (e.g., containing transcriptional activator and/or repressor proteins), transfecting and transducing cells with the nucleic acids, and monitoring cells for multimerization of fusion proteins and effects thereof (e.g., activation and/or repression of transcription) are described herein and/or known to those of skill in the art (see, e.g., Biggar and Crabtree (2000) J Biol Chem 275(33):25381-25390).

In another example of these methods provided herein, a cell is transfected or transduced with (1) nucleic acid encoding a fusion of an RNA binding-domain protein, or portion thereof, and a FKBP12 protein (or variant thereof) and (2) nucleic acid encoding a fusion of an mRNA splicing regulator protein (e.g., an SR protein), or RS domain thereof, and a FKBP12 protein (or variant thereof). The cell is then contacted with a compound provided herein, or pharmaceutically acceptable salts thereof, that binds to and multimerizes the FKBP12 proteins contained in the fusion proteins and may be monitored for pre-mRNA splicing and expression. This method is useful, for example, for regulation of protein expression. Methods of generating nucleic acid vectors for expression of fusion proteins, transfecting and transducing cells with the nucleic acids, and monitoring cells for multimerization of fusion proteins and effects thereof (e.g., transcription and mRNA splicing) are described herein and/known to those of skill in the art (see, e.g., Rivera et al. (1996) Nat Med 2:1028-1032; Graveley (2005) RNA 11:355-358).

### Expression Constructs

Expression constructs encode chimeric polypeptides comprising one or more multimerizing regions and at least one additional polypeptide, such as, for example, a Caspase-9 polypeptide, or a costimulating polypeptide, such as, for example, MyD88, CD40, or both MyD88 and CD40 polypeptides. The chimeric polypeptides expressed from such expression constructs may be contacted by a multimeric compound described herein, or a pharmaceutically acceptable salt thereof.

In certain embodiments, a chimeric polypeptide may comprise more than one ligand binding domain or multimerizing region. In some embodiments, the chimeric polypeptide may comprise one, two, three, or more ligand binding domains or multimerizing regions. An expression construct may or may not encode a membrane-targeting sequence. In some embodiments, the chimeric polypeptide may comprise a membrane targeting region. In other embodiments, the chimeric polypeptide does not include a membrane targeting region. Appropriate expression constructs may include a costimulating polypeptide region on either side of the ligand binding domain or multimerizing region. In some embodiments, the costimulating polypeptide region is provided at a location on the polypeptide that is amino terminal to the ligand binding domain or multimerizing region. In some embodiments, the ligand binding domain or multimerizing region is provided at a location on the polypeptide that is amino terminal to the costimulating polypeptide region.

For purposes of the present application, the terms “multimerizing region” “multimerization region” “ligand binding region” and “multimeric ligand binding region” are interchangeable.

In some embodiments, a nucleic acid that encodes a chimeric polypeptide may encode a heterologous protein (e.g., heterologous to an apoptosis-inducing polypeptide or costimulating polypeptide), non-limiting examples of which include a marker polypeptide, a chimeric antigen receptor, or a recombinant T cell receptor.

In some embodiments, the polypeptides may be expressed separately from the same vector, where each polynucleotide coding for one of the polypeptides is operably linked to a separate promoter. In certain embodiments, a promoter may be operably linked to each of the two polynucleotides, directing the production of two separate RNA transcripts, and thus two polypeptides. Therefore, the expression constructs discussed herein may comprise at least one, or at least two promoters.

A heterologous polypeptide, for example, a chimeric antigen receptor, may be linked to apoptosis-inducing polypeptide or costimulating polypeptide via a polypeptide sequence, such as, for example, a cleavable 2A-like sequence. For example, a nucleic acid that encodes the chimeric polypeptide may comprise a polynucleotide that encodes the chimeric polypeptide, a polynucleotide that encodes the 2A-like sequence, and a polynucleotide that encodes the heterologous polypeptide, with one promoter operably linked to the three polynucleotides. In such embodiments, the polypeptides are separated during translation, resulting in two polypeptides, such as, for example, the chimeric polypeptide that comprises the multimerizing region and the additional polypeptide, such as, for example the apoptosis-inducing polypeptide or the costimulating polypeptide, and the heterologous polypeptide, such as, for example, a chimeric antigen receptor polypeptide.

2A-like sequences, or “cleavable” 2A sequences, are derived from, for example, many different viruses, including, from Thosea asigna. These sequences are sometimes also known as “peptide skipping sequences.” When this type of sequence is placed within a cistron, between two polypeptides that are intended to be separated, the ribosome appears to skip a peptide bond, In the case of Thosea asigna sequence, the bond between the Gly and Pro amino acids is omitted. This leaves two polypeptides, for example, a Caspase-9 polypeptide and a marker polypeptide, or a chimeric antigen receptor polypeptide. When this sequence is used, the polypeptide that is encoded 5′ of the 2A sequence may end up with additional amino acids at the carboxy terminus, including the Gly residue and any upstream in the 2A sequence. The polypeptide that is encoded 3′ of the 2A sequence may end up with additional amino acids at the amino terminus, including the Pro residue and any downstream in the 2A sequence. “2A” or “2A-like” sequences are part of a large family of peptides that can cause peptide bond-skipping. Various 2A sequences have been characterized (e.g., F2A, P2A, T2A), and are examples of 2A-like sequences that may be used in the polypeptides of the present application. In certain embodiments, the 2A linker comprises the amino acid sequence of SEQ ID NO: 8; in certain embodiments the 2A linker consists of the amino acid sequence of SEQ ID NO: 8. In certain embodiments, the 2A linker further comprises a GSG amino acid sequence at the amino terminus of the polypeptide, in other embodiments, the 2A linker comprises a GSGPR amino acid sequence at the amino terminus of the polypeptide. Thus, by a “2A” sequence, the term may refer to a 2A sequence as provided herein, or may also refer to a 2A sequence as listed herein further comprising a GSG or GSGPR sequence at the amino terminus of the linker.

In certain embodiments, the chimeric polypeptide and a heterologous polypeptide may be expressed in a cell using two separate vectors. The cells may be co-transfected or co-transduced with the vectors, or the vectors may be introduced to the cells at different times.

In some embodiments, a nucleic acid is contained within a viral vector. In certain embodiments, the viral vector is a retroviral vector. In certain embodiments, the viral vector is an adenoviral vector or a lentiviral vector. It is understood that in some embodiments, the cell is contacted with the viral vector ex vivo, and in some embodiments, the cell is contacted with the viral vector in vivo. Thus, an expression construct may be inserted into a vector, for example a viral vector or plasmid. The steps of the methods provided may be performed using any suitable method; these methods include, without limitation, methods of transducing, transforming, or otherwise providing nucleic acid to the cell, provided herein.

Gene expression vector: The terms “gene expression vector”, “nucleic acid expression vector”, or “expression vector” as used herein, which can be used interchangeably throughout the document, generally refers to a nucleic acid molecule (e.g., a plasmid, phage, autonomously replicating sequence (ARS), artificial chromosome, yeast artificial chromosome (e.g., YAC)) that can be replicated in a host cell and be utilized to introduce a gene or genes into a host cell. The genes introduced on the expression vector can be endogenous genes (e.g., a gene normally found in the host cell or organism) or heterologous genes (e.g., genes not normally found in the genome or on extra-chromosomal nucleic acids of the host cell or organism). The genes introduced into a cell by an expression vector can be native genes or genes that have been modified or engineered. The gene expression vector also can be engineered to contain 5′ and 3′ untranslated regulatory sequences that sometimes can function as enhancer sequences, promoter regions and/or terminator sequences that can facilitate or enhance efficient transcription of the gene or genes carried on the expression vector. A gene expression vector sometimes also is engineered for replication and/or expression functionality (e.g., transcription and translation) in a particular cell type, cell location, or tissue type. Expression vectors sometimes include a selectable marker for maintenance of the vector in the host or recipient cell.

By “region “or “domain” is meant a polypeptide, or fragment thereof, that maintains the function of the polypeptide as it relates to the chimeric polypeptides of the present application. That is, for example, a FKBP12 binding domain, FKBP12 domain, FKBP12 region, FKBP12 multimerizing region, and the like, refer to a FKBP12 polypeptide that binds to the CID ligand or multimeric compound, such as, for example, rimiducid, or a multimeric compound described herein, to cause, or allow for, dimerization or multimerization of the chimeric polypeptide. By “region” or “domain” of a pro-apoptotic polypeptide, for example, the Caspase-9 polypeptides or truncated Caspase-9 polypeptides of the present application, is meant that upon dimerization or multimerization of the Caspase-9 region as part of the chimeric polypeptide, or chimeric pro-apoptotic polypeptide, the dimerized or multimerized chimeric polypeptide can participate in the caspase cascade, allowing for, or causing, apoptosis.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct. As used herein, the terms “treatment”, “treat”, “treated”, or “treating” refer to prophylaxis and/or therapy.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra.

As used herein, the term “gene” is defined as a functional protein, polypeptide, or peptide-encoding unit. As will be understood, this functional term includes genomic sequences, cDNA sequences, and smaller engineered gene segments that express, or are adapted to express, proteins, polypeptides, domains, peptides, fusion proteins, and mutants.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

As used herein, the term “polynucleotide” is defined as a chain of nucleotides. Furthermore, nucleic acids are polymers of nucleotides. Thus, nucleic acids and polynucleotides as used herein are interchangeable. Nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

As used herein, the term “polypeptide” is defined as a chain of amino acid residues, usually having a defined sequence. As used herein the term polypeptide is interchangeable with the terms “peptides” and “proteins”.

Amino acids other than those indicated as conserved may differ in a protein or enzyme so that the percent protein or amino acid sequence similarity between any two proteins of similar function may vary and can be, for example, at least 70%, at least 80%, at least 90%, and at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the settings may be selected that result in the highest sequence similarity.

As used herein, the term “promoter” is defined as a DNA sequence recognized by the synthetic machinery of the cell, or introduced synthetic machinery, required to initiate the specific transcription of a gene.

In some embodiments, the promoter is a developmentally regulated promoter. The term “developmentally regulated promoter” as used herein refers to a promoter that acts as the initial binding site for RNA polymerase to transcribe a gene which is expressed under certain conditions that are controlled, initiated by or influenced by a developmental program or pathway. Developmentally regulated promoters often have additional control regions at or near the promoter region for binding activators or repressors of transcription that can influence transcription of a gene that is part of a development program or pathway. Developmentally regulated promoters sometimes are involved in transcribing genes whose gene products influence the developmental differentiation of cells. A developmentally regulated promoter may be used in the nucleic acids of the present application, where it is anticipated that the nucleic acid will be expressed in developmentally differentiated cells.

The term “developmentally differentiated cells”, as used herein refers to cells that have undergone a process, often involving expression of specific developmentally regulated genes, by which the cell evolves from a less specialized form to a more specialized form in order to perform a specific function. Non-limiting examples of developmentally differentiated cells are liver cells, lung cells, skin cells, nerve cells, blood cells, and the like. Changes in developmental differentiation generally involve changes in gene expression (e.g., changes in patterns of gene expression), genetic re-organization (e.g., remodeling or chromatin to hide or expose genes that will be silenced or expressed, respectively), and occasionally involve changes in DNA sequences (e.g., immune diversity differentiation). Cellular differentiation during development can be understood as the result of a gene regulatory network. A regulatory gene and its cis-regulatory modules are nodes in a gene regulatory network that receive input (e.g., protein expressed upstream in a development pathway or program) and create output elsewhere in the network (e.g., the expressed gene product acts on other genes downstream in the developmental pathway or program).

The term “transfection” and “transduction” are interchangeable and refer to the process by which an exogenous DNA sequence is introduced into a eukaryotic host cell. Transfection (or transduction) can be achieved by any one of a number of means including electroporation, microinjection, gene gun delivery, retroviral infection, lipofection, superfection and the like.

As used herein, the term “under transcriptional control,” “operably linked,” or “operatively linked” is defined as the promoter is in the correct location and orientation in relation to the nucleic acid to control RNA polymerase initiation and expression of the gene. In general, the term “operably linked” is meant to indicate that the promoter sequence is functionally linked to a second sequence, wherein, for example, the promoter sequence initiates and mediates transcription of the DNA corresponding to the second sequence.

### Pharmaceutical Compositions and Formulations

A multimeric compound described herein can be prepared as a pharmaceutically acceptable salt. As used herein, the term “pharmaceutically acceptable salt” refers to a derivative of the disclosed compounds where the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include conventional non-toxic salts or quaternary ammonium salts of the parent compound formed, for example, from non-toxic inorganic or organic acids. For example, conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. In certain examples, conventional non-toxic salts include those derived from bases, such as potassium hydroxide, sodium hydroxide, ammonium hydroxide, caffeine, various amines, and the like.

A pharmaceutically acceptable salt can be prepared from a parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, for example, nonaqueous media like ether, ethyl acetate, ethanol, isopropanol, or acetonitrile. Lists of suitable salts are found in Remington's Pharmaceutical Sciences, 17th ed., Mack Publishing Company, Easton, Pa., p. 1418 (1985), the disclosure of which is hereby incorporated by reference herein in its entirety for all purposes.

The term “pharmaceutically acceptable” or “pharmacologically acceptable” as used herein refers to compounds, materials, compositions, and/or dosage forms which are, within the scope of sound medical judgment, suitable for administration to humans or animals and suitable for use in contact with the tissues of human beings and animals without excessive toxicity, irritation, allergic response, or other problem or complication commensurate with a reasonable benefit/risk ratio. Moreover, for human administration, preparations may meet sterility, pyrogenicity, and general safety and purity standards as required by FDA Office of Biologics standards.

A multimeric compound described herein often is a stable compound and often has a stable structure in a composition provided. The terms “stable compound” and “stable structure” are meant to indicate a compound that is sufficiently robust to survive isolation to a useful degree of purity from a reaction mixture, and formulation into an efficacious therapeutic agent. Stable compounds are contemplated herein for use in treatment methods described.

In some embodiments, provided herein are pharmaceutical compositions comprising a multimeric compound described herein, or a pharmaceutically acceptable salt thereof. In addition to a multimeric compound or pharmaceutically acceptable salt thereof, a pharmaceutical composition can include one or more of a pharmaceutically acceptable excipient, carrier, solvent, diluent, isotonic agent, buffering agent, stabilizer, preservative, vaso-constrictive agent, antibacterial agent, antifungal agent, and the like, for example. Non-limiting examples of solvents, and diluents include water, saline, dextrose, ethanol, glycerol, oil, and the like. Examples of isotonic agents include sodium chloride, dextrose, mannitol, sorbitol, lactose, and the like. Useful stabilizers include gelatin, albumin, and the like. A pharmaceutically-acceptable carrier includes any and all solvents, dispersion media, coatings, stabilizing agents, diluents, preservatives, antibacterial and antifungal agents, isotonic agents, adsorption delaying agents, and the like. Carrier(s) generally are compatible with other components of the multimeric compound pharmaceutical composition and not deleterious to a subject when administered. A carrier often is sterile and pyrogen-free, and selected based on the mode of administration used, and a carrier utilized often is approved, or will be approved, by an appropriate government agency that oversees development and use of pharmaceuticals. A pharmaceutical composition can include, in certain embodiments, a compatible pharmaceutically acceptable (i.e., sterile or non-toxic) liquid, semisolid, or solid diluent that serves as a pharmaceutical vehicle, excipient, or medium. A diluent can include water, saline, dextrose, ethanol, glycerol, and the like, for example. An isotonic agent can include sodium chloride, dextrose, mannitol, sorbitol, and lactose, among others. A stabilizer can include albumin, among others. A pharmaceutical composition can include, in some embodiments, an antibiotic or preservative, including, for example, gentamicin, merthiolate, or chlorocresol. In some embodiments, an excipient or carrier is chosen from polyethylene glycol (PEG), polysorbate, ethanol, glycerol, glycerin, sorbitol, glucose, sucrose, dimethylacetamide, triacetin, dimethylsulfoxide (DMSO), and an oil, such as a vegetable oil, and combinations thereof. In some embodiments, an excipient or carrier is selected from the group consisting of polyethylene glycol (PEG), polysorbate, ethanol, glycerol, glycerin, sorbitol, glucose, sucrose, dimethylacetamide, triacetin, dimethylsulfoxide (DMSO), and an oil, such as a vegetable oil, and combinations thereof. Various sustained release systems for drugs have also been devised, and can be applied to a compound described herein. See, for example, U.S. Pat. No. 5,624,677, the methods of which are incorporated herein by reference in its entirety for all purposes.

In some embodiments, a pharmaceutical composition is a liquid composition. In some embodiments, a pharmaceutical composition is provided as a dry powder composition. In some embodiments, a pharmaceutical composition is in a liposomal composition, sometimes as a micro-emulsion. In some embodiments, a pharmaceutical composition is a spray dried composition. In some embodiments, a pharmaceutical composition comprises a pharmaceutically acceptable co-polymer. In some embodiments, the co-polymer is chosen from, or selected from the group consisting of, poly(vinyl alcohol), poly(vinyl pyrrolidone), hypromellose, acetate, and succinate, and combinations thereof.

In the case of sterile powders for the preparation of sterile injectable solutions, preparation methods sometimes utilized are vacuum drying and the freeze drying techniques, which yield a powder of a multimeric compound described herein or pharmaceutically acceptable salt thereof in addition to any additional desired ingredient present in the previously sterile-filtered solutions. In some embodiments, the multimeric compounds described herein, or pharmaceutically acceptable salts thereof, are provided in a spray dried form. A dry powder or spray dried powder may be provided for shipping of the multimeric compound described herein, or a pharmaceutically acceptable salt thereof. A dry powder or spray dried powder may be dissolved in water, buffered water, saline or buffered saline with or without a co-solvent, for use.

For spray drying, a feed solution comprising a multimeric compound described herein can include a co-polymer, non-limiting examples of which include poly(vinyl alcohol), poly(vinyl pyrrolidone), hypromellose acetate succinate, and combinations thereof. A dry powder formulation also can contain a co-polymer in some embodiments.

Water or saline used for preparing a pharmaceutical composition may be buffered or not buffered. Non-limiting examples of saline solutions that can be used to prepare a pharmaceutical composition include lactated Ringer's solution, acetated Ringer's solution, intravenous sugar solutions (e.g., 5% dextrose in normal saline (D5NS), 10% dextrose in normal saline (D10NS), 5% dextrose in half-normal saline (D5HNS) and 10% dextrose in half-normal saline (D10HNS)). Non-limiting example of buffered saline solutions and related solutions include phosphate buffered saline (PBS), TRIS-buffered saline (TBS), Hank's balanced salt solution (HBSS), Earle's balanced salt solution (EBSS), standard saline citrate (SSC), HEPES-buffered saline (HBS), and Gey's balanced salt solution (GBSS).

A multimeric compound described herein or pharmaceutically acceptable salt thereof can be provided in a pharmaceutical dosage form. A pharmaceutical dosage form can include a sterile aqueous solution or dispersion or sterile powder comprising a multimeric compound described herein or pharmaceutically acceptable salt thereof, which are adapted for the extemporaneous preparation of sterile solutions or dispersions, and optionally encapsulated in liposomes. The ultimate dosage form sometimes is a sterile fluid and stable under the conditions of manufacture and storage. A liquid carrier or vehicle can be a solvent or liquid dispersion medium comprising, for example, water, saline, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. An isotonic agent, for example, a sugar, buffer or sodium chloride is included in some embodiments. Prolonged absorption of an injectable composition can be brought about by the use in the compositions of agents delaying absorption, for example, aluminum monostearate and gelatin. Sterile solutions often are prepared by incorporating an active compound in a required amount in an appropriate solvent, sometimes with one or more of the other ingredients enumerated above, followed by filter sterilization.

In some embodiments, a liquid pharmaceutical composition has an 80% (weight/weight) or greater amount of a multimeric compound described herein or a pharmaceutically acceptable salt thereof in water. In some embodiments, the concentration of a compound described herein in a liquid composition is about 0.1-25% (weight/weight), and sometimes about 0.5-10% (weight/weight). The concentration in a semi-solid or solid composition such as a gel or a powder sometimes is about 0.1-5% (weight/weight), and sometimes about 0.5-2.5% (weight/weight).

In some embodiments, a multimeric compound is provided at 0.4 mg/kg per dose, for example at a concentration of 5 mg/mL. Vials or other containers may be provided containing the multimeric compound at, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

A multimeric compound described herein or pharmaceutically acceptable salt thereof can be formulated in combination with one or more other pharmaceutically active agents. The one or more other agents can include, without limitation, another compound described herein, an anti-cell proliferative agent (e.g., chemotherapeutic), an anti-inflammatory agent, or an antigen.

### Administration of Multimeric Compounds

Upon formulation, solutions and solid forms of multimeric compounds described herein, or pharmaceutically acceptable salts thereof, can be administered in a manner compatible with the dosage formulation and in such amount as is therapeutically effective. The formulations can be administered in a variety of dosage forms, dependent on the method of administration. Some variation in dosage will necessarily occur depending on the condition of the subject being treated. The person responsible for administration will, in any event, determine the appropriate dose for the individual subject.

A multimeric compound described herein can be formulated as a pharmaceutical composition and administered to a mammalian host, such as a human patient or nonhuman animal, in a variety of forms adapted to the chosen route of administration. The terms “patient” or “subject” are interchangeable, and, as used herein include, but are not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

The active compositions may include classic pharmaceutical preparations. Administration of these compositions can be by any common route so long as the target tissue is available via that route. Non-limiting examples of administration routes include oral, nasal, buccal, rectal, vaginal, topical, orthotopic, intradermal, instillation (e.g., bladder instillation, parenteral, subcutaneous, intravascular, intramuscular, intraperitoneal or intravenous injection or infusion. In certain embodiments, the multimeric compounds described herein, or pharmaceutically acceptable salts thereof, are administered by intravenous injection or infusion. Such compositions would normally be administered as pharmaceutically acceptable compositions, discussed herein.

In certain embodiments, a composition described herein is administered in conjunction with locally applied ultrasound, electromagnetic radiation or electroporation or other electrically based drug delivery technique, local chemical abrasion, or local physical abrasion.

Useful dosages of compounds can be determined by comparing their in vitro activity, and in vivo activity in animal models. It is understood that methods are available for the extrapolation of effective dosages in mice, and other animals, to humans.

The amount of the compound, or an active salt or derivative thereof, required for use in treatment varies not only with a particular salt selected but also with the route of administration, the nature of the condition being treated and the age and condition of the patient and will be ultimately at the discretion of the attendant physician or clinician. In general a suitable dose sometimes is in the range of from about 0.1 to about 100 mg/kg, e.g., from about 0.1 to about 50 mg/kg of subject body weight per day, from about 0.1 to about 10 mg per kilogram subject body weight of the recipient per day, such as from about 0.2 to 4 mg/kg of subject body weight per day, and often is in the range of 0.3 to 1 mg/kg of subject body weight per day, such as, for example, about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject body weight per day. A compound may be conveniently administered in unit dosage form, and for example, contain 5 to 1000 mg, or 10 to 750 mg, or 50 to 500 mg of multimeric compound described herein or pharmaceutically acceptable salt thereof per unit dosage form. An multimeric compound described herein or pharmaceutically acceptable salt thereof can be administered to achieve peak plasma concentrations of an active compound of from about 0.01 to about 100 pM, about 0.5 to about 75 pM, about 1 to 50 pM, or about 2 to about 30 pM. Such concentrations may be achieved, for example, by the intravenous injection of a 0.05 to 5% solution of a multimeric compound described herein or pharmaceutically acceptable salt thereof, optionally in saline, or orally administered as a bolus containing about 1-100 mg of a multimeric compound described herein or pharmaceutically acceptable salt thereof. Desirable blood levels may be maintained by continuous infusion to provide about 0.01-5.0 mg/kg/hr or by intermittent infusions containing about 0.4-15 mg/kg of the multimeric compound described herein or pharmaceutically acceptable salt thereof. A desired dose may conveniently be presented in a single dose or as divided doses administered at appropriate intervals, for example, as two, three, four or more sub-doses per day. A sub-dose itself may be further divided, e.g., into a number of discrete loosely spaced administrations; such as multiple inhalations from an insufflator or by application of a plurality of drops into the eye.

In some examples, a multimeric compound described herein or pharmaceutically acceptable salt thereof may be prepared and administered according to methods used to prepare and administer rimiducid, an example of which is described hereafter.

Administration of Compound A and Other Multimeric Compounds

As noted above, multimeric compounds described herein, including, for example, Compound A, or pharmaceutically acceptable salts thereof, can be formulated in the same or a similar formulation as for rimiducid. Formulations described hereafter can be used for injection or intravenous administration, and sometimes for another route of administration. An example of a formulation and administration of rimiducid is provided herein. Rimiducid (AP1903) has a low level of solubility in aqueous solution, and is therefore often formulated in a non-ionic solubilizer, such as Solutol. Rimiducid (AP1903) is manufactured by Alphora Research Inc. and AP1903 Drug Product for Injection is made by Alcami Corporation (Durham, N.C.) Inc. It is formulated as a 5 mg/mL solution of AP1903 in a 25% solution of the non-ionic solubilizer Solutol HS 15 (250 mg/mL, BASF). At room temperature, this formulation is a clear, slightly yellow solution. Upon refrigeration, this formulation undergoes a reversible phase transition, resulting in a milky solution. This phase transition is reversed upon re-warming to room temperature. The fill is 2.33 mL in a 3 mL glass vial (˜10 mg AP1903 for Injection total per vial).

AP1903 is removed from the refrigerator the night before the patient is dosed and stored at a temperature of approximately 21° C. overnight, so that the solution is clear prior to dilution. The solution is prepared within 30 minutes of the start of the infusion in glass or polyethylene bottles or non-DEHP bags and stored at approximately 21° C. prior to dosing.

All study medication is maintained at a temperature between 2° C. and 8° C., protected from excessive light and heat, and stored in a locked area with restricted access.

Upon determining a need to administer AP1903, patients may, for example, be administered a single fixed dose of AP1903 for Injection (0.4 mg/kg) via IV infusion over 2 hours, using a non-DEHP, non-ethylene oxide sterilized infusion set. The dose of AP1903 is calculated individually for all patients, and is not to be recalculated unless subject body weight fluctuates by ≥10%. The calculated dose is diluted in 100 mL in 0.9% normal saline before infusion.

In a previous Phase 1 study of AP1903, 24 healthy volunteers were treated with single doses of AP1903 for Injection at dose levels of 0.01, 0.05, 0.1, 0.5 and 1.0 mg/kg infused IV over 2 hours. AP1903 plasma levels were directly proportional to dose, with mean Cmax values ranging from approximately 10-1275 ng/mL over the 0.01-1.0 mg/kg dose range. Following the initial infusion period, blood concentrations demonstrated a rapid distribution phase, with plasma levels reduced to approximately 18, 7, and 1% of maximal concentration at 0.5, 2 and 10 hours post-dose, respectively. AP1903 for Injection was shown to be safe and well tolerated at all dose levels and demonstrated a favorable pharmacokinetic profile. Iuliucci J D, et al., J Clin Pharmacol. 41: 870-9, 2001.

The fixed dose of AP1903 for injection used, for example, may be 0.4 mg/kg intravenously infused over 2 hours. The amount of AP1903 needed in vitro for effective signaling of cells is 10-100 nM (1600 Da MVV). This equates to 16-160 μg/L or ˜0.016-1.6 mg/kg (1.6-160 μg/kg). Doses up to 1 mg/kg were well-tolerated in the Phase 1 study of AP1903 discussed above. Therefore, 0.4 mg/kg may be a safe and effective dose of AP1903 for this Phase I study in combination with the therapeutic cells.

Components of a pharmaceutical composition often depend on its intended administration route, and non-limiting examples of which are provided hereafter.

Intravenous Administration

A multimeric compound described herein such as, for example, COMPOUND A, or a pharmaceutically acceptable salt thereof, may be formulated in an aqueous solution, in water, saline, in the presence or absence of buffers, stabilizers, nontoxic surfactants, excipients, carriers, and the like, as discussed herein. Non-limiting examples of buffers, such as, for example weak buffers that may be provided in the solution include acetate, phosphate, citrate, and the like, such that the solution containing the multimeric compound is less than pH 4, pH5, pH6, or pH7. In some embodiments, multimeric compounds described herein, or pharmaceutically acceptable salts thereof, are formulated in a 0.1% glycerol solution. In some embodiments, a multimeric compound described herein is formulated in water, saline or related solution with 0.05, 0.1, 0.15, 0.2, 0.25, 0.3, 0.35, or 0.4% glycerol. In some embodiments, a multimeric compound is formulated in water, saline or a related solution with 0.5, 0.1, 0.15, 0.2, 0.25 0.3, 0.35, or 0.4% ethanol.

Administration by Injection

Pharmaceutical forms suitable for injectable use include forms suitable for intravenous administration discussed herein, modified as appropriate for injections, and also include pharmaceutical forms in sterile aqueous solutions or dispersions and sterile powders for the extemporaneous preparation of sterile injectable solutions or dispersions. An injectable formulation often is sterile and is fluid to the extent that easy syringeability exists. It is stable under the conditions of manufacture and storage and is preserved against the contaminating action of microorganisms, such as bacteria and fungi.

An injectable formulation sometimes includes a carrier, which can be a solvent, excipient, or dispersion medium. Fluidity of an injectable formulation can be maintained, for example, by the use of a coating, such as lecithin, by the maintenance of the required particle size in the case of dispersion and by the use of surfactants. Prevention of the action of microorganisms can be effected by various antibacterial and antifungal agents, for example, parabens, chlorobutanol, phenol, sorbic acid, thimerosal, and the like. In certain examples, isotonic agents, for example, sugars or sodium chloride may be included. Prolonged absorption of an injectable compositions can be effected by the use in the compositions of agents delaying absorption, for example, aluminum monostearate and gelatin. The pharmaceutical form may comprise a co-polymer such as, for example, a co-polymer chosen from group poly(vinyl alcohol), poly(vinyl pyrrolidone), and hypromellose acetate succinate. The pharmaceutical form may comprise a co-polymer such as, for example, a co-polymer selected from the group consisting of poly(vinyl alcohol), poly(vinyl pyrrolidone), and hypromellose acetate succinate.

Oral Administration

Multimeric compounds described herein, or pharmaceutically acceptable salts thereof, described herein may be enclosed in hard or soft shell gelatin capsules, may be compressed into tablets, or may be incorporated directly with the food of the patient's diet. For oral therapeutic administration, an active compound may be combined with one or more excipients and used in the form of ingestible tablets, buccal tablets, troches, capsules, elixirs, suspensions, syrups, wafers, and the like. Such compositions and preparations sometimes contain at least 0.1% of active compound. The percentage of the compositions and preparations may be varied and sometimes are about 2% to about 60% of the weight of a given unit dosage form. The amount of active compound in such therapeutically useful compositions is such that an effective dosage level will be obtained.

Tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Topical Administration

For embodiments pertaining to topical administration, a multimeric compound described herein or pharmaceutically acceptable salt thereof may be applied in pure form, e.g., when in liquid form. However, it is generally desirable to administer a compound as a composition or formulation, in combination with an acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, or phospholipids in propylene glycol/ethylene glycol, in which the present compounds can be dissolved or dispersed at effective levels, optionally with the aid of non-toxic surfactants. A composition sometimes includes a diluent and sometimes an adjuvant, carrier (e.g., assimilable, editable), buffer, preservative and the like. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

### Methods of Treatment

Provided in certain embodiments are methods of treatment or prevention of a disease where the methods comprise contacting a cell that expresses a chimeric polypeptide comprising a multimerizing region, with a multimeric compound described herein, or a pharmaceutically acceptable salt thereof that binds to the multimerizing region, resulting in multimerization of the chimeric polypeptide. The cells may be contacted with the multimeric compound described herein, or a pharmaceutically acceptable salt thereof ex vivo, or in vivo. As used herein, the term “ex vivo” refers to “outside” the body. The terms “ex vivo” and “in vitro” can be used interchangeably herein. In some embodiments, modified cells that express the chimeric polypeptide are administered to a subject before, or at the same time that the multimeric compound described herein, or a pharmaceutically acceptable salt thereof is administered to the subject. In some embodiments, the multimeric compound described herein, or a pharmaceutically acceptable salt thereof is administered to a subject, wherein modified cells that express the chimeric polypeptide have been administered to the subject. In such embodiments, the multimeric compound described herein, or a pharmaceutically acceptable salt thereof is administered to a subject who has received a transfusion or other administration of modified cells, where the modified cells express a chimeric protein comprising a multimerizing region that binds to the multimeric compound described herein, or a pharmaceutically acceptable salt thereof. The chimeric polypeptide may comprise, for example, an apoptotic polypeptide, such as Caspase-9, or a Caspase-9 polypeptide that lacks the CARD domain. In other embodiments, the chimeric polypeptide may comprise a polypeptide that activates cell activity, for example, immune activity, such as, for example, a costimulating polypeptide.

Cells, such as, for example, T cells, tumor infiltrating lymphocytes, natural killer cells, natural killer T cells, or progenitor cells, such as, for example, hematopoietic stem cells, mesenchymal stromal cells, stem cells, pluripotent stem cells, and embryonic stem cells may be used for cell therapy.

The cells may be from a donor, or may be cells obtained from the patient. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. Mesenchymal stromal cells have also, for example, been used to provide immunosuppressive activity, and may be used in the treatment of graft versus host disease and autoimmune disorders.

By “therapeutic cell” is meant a cell used for cell therapy, that is, a cell administered to a subject to treat or prevent a condition or disease. In some embodiments, there is a need to eliminate, or reduce the number of therapeutic cells in a subject. In certain embodiments, the therapeutic cells express a chimeric polypeptide comprising a multimerizing region and an apoptotic polypeptide, for example, a FKBP12v36 multimerizing region and a Caspase-9 polypeptide, and the number of therapeutic cells may be reduced by administering a multimeric compound described herein, or a pharmaceutically acceptable salt thereof to the subject.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

By “obtained or prepared” as, for example, in the case of cells, is meant that the cells or cell culture are isolated, purified, or partially purified from the source, where the source may be, for example, umbilical cord blood, bone marrow, or peripheral blood. The terms may also apply to the case where the original source, or a cell culture, has been cultured and the cells have replicated, and where the progeny cells are now derived from the original source.

In other examples, T cells are used to treat various diseases and conditions, and as a part of stem cell transplantation. An adverse event that may occur after haploidentical T cell transplantation is graft versus host disease (GvHD). The likelihood of GvHD occurring increases with the increased number of T cells that are transplanted. This limits the number of T cells that may be infused. By having the ability to selectively remove the infused T cells in the event of GvHD in the patient, a greater number of T cells may be infused, increasing the number to greater than 10⁶, greater than 10⁷, greater than 10⁸, or greater than 10⁹ cells. The number of T cells/kg subject body weight that may be administered may be, for example, from about 1×10⁴ T cells/kg subject body weight to about 9×10⁷ T cells/kg subject body weight, for example about 1, 2, 3, 4, 5, 6, 7, 8, or 9×10⁴; about 1, 2, 3, 4, 5, 6, 7, 8, or 9×10⁵; about 1, 2, 3, 4, 5, 6, 7, 8, or 9×10⁶; or about 1, 2, 3, 4, 5, 6, 7, 8, or 9×10⁷ T cells/kg subject body weight. In other examples, therapeutic cells other than T cells may be used. The number of therapeutic cells/kg body weight that may be administered may be, for example, from about 1×10⁴ T cells/kg body weight to about 9×10⁷ T cells/kg body weight, for example about 1, 2, 3, 4, 5, 6, 7, 8, or 9×10⁴; about 1, 2, 3, 4, 5, 6, 7, 8, or 9×10⁵; about 1, 2, 3, 4, 5, 6, 7, 8, or 9×10⁶; or about 1, 2, 3, 4, 5, 6, 7, 8, or 9×10⁷ therapeutic cells/kg body weight.

The term “unit dose” as it pertains to the inoculum refers to physically discrete units suitable as unitary dosages for mammals, each unit containing a predetermined quantity of pharmaceutical composition calculated to produce the desired immunogenic effect in association with the required diluent. The specifications for the unit dose of an inoculum are dictated by and are dependent upon the unique characteristics of the pharmaceutical composition and the particular immunologic effect to be achieved.

An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

For eliminating therapeutic cells, an effective amount of the pharmaceutical composition that comprises multimeric compounds described herein, or pharmaceutically acceptable salts thereof, would be the amount that achieves the selected result of selectively removing the cells that express an inducible chimeric apoptotic polypeptide, such as a chimeric polypeptide that comprises a multimerizing region and a Caspase-9 polypeptide lacking the CARD domain, such that over 60%, 70%, 80%, 85%, 90%, 95%, or 97% of the Caspase-9 expressing cells are killed. The term is also synonymous with “sufficient amount.”

For activating the immune activity of therapeutic cells, for example, where the therapeutic cells express an inducible chimeric costimulatory polypeptide and a chimeric antigen receptor that binds to a target cell, such as, for example, a tumor cell, an effective amount of the pharmaceutical composition that comprises a multimeric compound described herein, or a pharmaceutically acceptable salt thereof, would be the amount that achieves the selective result of reduces the number of target cells, by over 30%, 40%, 50%, 60%, 70%, 80%, 85%, 90%, 95%, or 97%. The term is also synonymous with “sufficient amount.”

For activating the immune activity of therapeutic cells, an effective amount of the pharmaceutical composition that comprises a multimeric compound described herein, or a pharmaceutically acceptable salt thereof may be, for example, the amount that increases or decreases biological activity as measured in a biological assay for immune cell activation, such as, for example, a SEAP assay, or increases or decreases the presence of a biological marker, where the increase or decrease in the biological activity, or the increase or decrease of the biological marker is associated with an activation of immune activity of the cell, by over 30%, 40%, 50%, 60%, 70%, 80%, 85%, 90%, 95%, or 97%. The term is also synonymous with “sufficient amount.”

A “number of target cells” may refer to an actual number of target cells, such as, for example, in a representative sample. In some examples, this number may be obtained from a sample taken before administration of the multimeric compound described herein, or a pharmaceutically acceptable salt thereof, and from a sample taken following administration of the compound. The sample may be of any appropriate tissue or bodily fluid that might provide a representative sampling of the number of target cells. In some examples, the term may refer to the size of a tumor, or the number of tumors present in an organ or tissue. In this example, the number of target cells is considered to be reduced where the size of the tumor, or the number of tumors is reduced following administration of the compound.

The effective amount for any particular application can vary depending on such factors as the disease or condition being treated, the particular composition being administered, the size of the subject, and/or the severity of the disease or condition, and the inducible chimeric polypeptide. One can empirically determine the effective amount of a particular composition provided herein without necessitating undue experimentation.

The terms “contacted” and “exposed,” when applied to a cell, tissue or organism, are used herein to discuss the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing. By “kill” or “killing” as in a percent of cells killed, is meant the death of a cell through apoptosis, as measured using any method known for measuring apoptosis, and, for example, using the assays discussed herein, such as, for example the SEAP assays or T cell assays discussed herein. The term may also refer to cell ablation.

The administration of the pharmaceutical composition may precede, be co-current with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within of from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition provided herein and one or more agents may be employed.

### Optimized and Personalized Therapeutic Treatment

The administration of the multimeric compounds described herein, or pharmaceutically acceptable salts thereof, may be optimized based on, for example, the disease or condition being treated or prevented, the patient's health, or other physical characteristics of the patient, or the desired outcome. Provided herein is an example of treatment of patients with rimiducid, following the induction of Graft vs Host Disease, where the therapeutic cells that express a chimeric polypeptide comprising an apoptosis-inducing polypeptide have been administered to the patient.

The induction of apoptosis after administration of rimiducid may be optimized by determining the stage of a negative side effect of the therapeutic cells, such as Graft vs Host Disease, or the number of undesired therapeutic cells that remain in the patient. Similarly, the activation of immune activity, such as activating cells that express a chimeric antigen receptor in addition to the inducible chimeric polypeptide, may be optimized by determining the number of target cells remaining in the subject, or by measuring a marker of immune activity, such as, for example, the secretion of certain cytokines or other markers.

For example, determining that a patient has GvHD, and the stage of the GvHD, provides an indication to a clinician that it may be necessary to induce Caspase-9 associated apoptosis by administering the multimeric compound described herein, or a pharmaceutically acceptable salt thereof. In another example, determining that a patient has a reduced level of GvHD after treatment with the multimeric compound described herein, or a pharmaceutically acceptable salt thereof may indicate to the clinician that no additional dose of the compound is needed. Similarly, after treatment with the multimeric compound described herein, or a pharmaceutically acceptable salt thereof, determining that the patient continues to exhibit GvHD symptoms, or suffers a relapse of GvHD may indicate to the clinician that it may be necessary to administer at least one additional dose of the compound. The term “dosage” is meant to include both the amount of the dose and the frequency of administration, such as, for example, the timing of the next dose.

In other embodiments, following administration of therapeutic cells, for example, therapeutic cells which express a chimeric antigen receptor in addition to the inducible Caspase-9 polypeptide, in the event of a need to reduce the number of modified cells or in vivo modified cells, the multimeric compound described herein, or a pharmaceutically acceptable salt thereof may be administered to the patient. In these embodiments, the methods comprise determining the presence or absence of a negative symptom or condition, such as Graft vs Host Disease, or off target toxicity, and administering a dose of a multimeric compound provided herein, such as, for example, Compound A. The methods may further comprise monitoring the symptom or condition and administering an additional dose of the multimeric compound described herein, or a pharmaceutically acceptable salt thereof in the event the symptom or condition persists. This monitoring and treatment schedule may continue while the therapeutic cells that express chimeric antigen receptors or chimeric signaling molecules remain in the patient.

In other embodiments, following administration of therapeutic cells, for example, therapeutic cells which express a chimeric antigen receptor in addition to the inducible costimulating polypeptide that activates immune cells, in order to induce the activity of the cell to reduce the number of target cells, such as tumor cells, the multimeric compound described herein, or a pharmaceutically acceptable salt thereof may be administered to the patient. In these embodiments, the methods comprise determining the number of target cells, and administering a dose of the multimeric compound described herein, or a pharmaceutically acceptable salt thereof to reduce the number of the target cells. The methods may further comprise monitoring a symptom or condition associated with the presence of the target cells and administering an additional dose of the multimeric compound described herein, or a pharmaceutically acceptable salt thereof in the event the symptom or condition persists. For example, the tumor load, tumor burden, amount of tumor cells, concentration of tumor cells, size of tumors, amount of cancerous or precancerous cells, concentration of cancerous or precancerous cells in the subject may be determined, and a dose of the multimeric compound described herein, or a pharmaceutically acceptable salt thereof is administered to reduce the number or concentration of tumor, cancerous, or precancerous cells.

An indication of adjusting or maintaining a subsequent drug dose, such as, for example, a subsequence dose of the multimeric compound described herein, or a pharmaceutically acceptable salt thereof, and/or the subsequent drug dosage, can be provided in any convenient manner. An indication may be provided in tabular form (e.g., in a physical or electronic medium) in some embodiments. For example, the graft versus host disease observed symptoms may be provided in a table, and a clinician may compare the symptoms with a list or table of stages of the disease. Or, for example, the tumor load, tumor burden, amount of tumor cells, concentration of tumor cells, size of tumors, amount of cancerous or precancerous cells, concentration of cancerous or precancerous cells in the subject may be provided in a table. The clinician then can identify from the table an indication for subsequent drug dose. For example, this information can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

In some examples, a dose, or multiple doses of the multimeric compound described herein, or a pharmaceutically acceptable salt thereof may be administered before clinical manifestations of GvHD, or other symptoms, such as CRS symptoms, are apparent. In this example, cell therapy is terminated before the appearance of negative symptoms. In other embodiments, such as, for example, hematopoietic cell transplant for the treatment of a genetic disease, the therapy may be terminated after the transplant has made progress toward engraftment, but before clinically observable GvHD, or other negative symptoms, can occur. In other examples, the multimeric compound described herein, or a pharmaceutically acceptable salt thereof may be administered to eliminate the modified cells in order to eliminate on target/off-tumor cells, such as, for example, healthy B cells co-expressing the B cell-associated target antigen.

### EXAMPLES

The examples set forth below illustrate certain embodiments and do not limit the technology.

### Example 1: Synthesis of Compound A

Compound A may be prepared following methods provided herein. The present Example provides a method of preparation according to Method 3 herein.

Synthetic example for obtaining a compound 15 where R_{L} is ethylene and R¹ and R² are methyl groups is shown below.

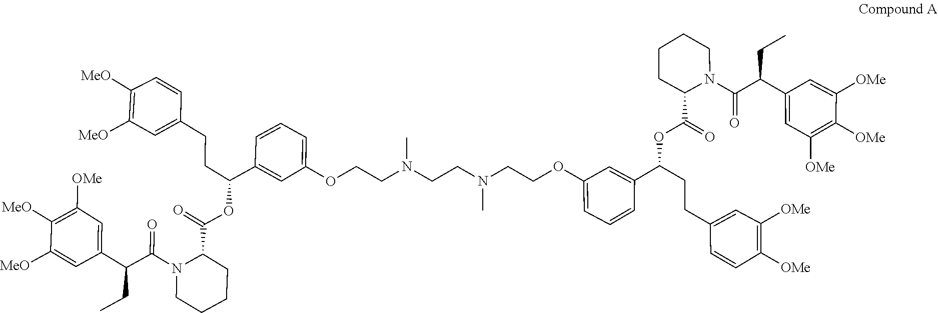 [SMILES: CC[C@H](C(=O)N1CCCC[C@H]1C(=O)O[C@H](CCc1ccc(OC)c(OC)c1)c1cccc(OCCN(C)CCN(C)CCOc2cccc([C@@H](CCc3ccc(OC)c(OC)c3)OC(=O)[C@@H]3CCCCN3C(=O)[C@@H](CC)c3cc(OC)c(OC)c(OC)c3)c2)c1)c1cc(OC)c(OC)c(OC)c1]

### Compound A—(1R,1′R)-((((ethane-1,2-diylbis(methylazanediyl))bis(ethane-2,1-diyl))bis(oxy))bis(3,1-phenylene))bis(3-(3,4-dimethoxyphenyl)propane-1,1-diyl) (2S,2′S)-bis(1-((S)-2-(3,4,5-trimethoxyphenyl)butanoyl)piperidine-2-carboxylate)

A suspension of LiAlH₄ (1.41 g, 37.18 mmol, 0.2 vol.) in THF (tetrahydrofuran) (74 mL, 9.0 vol.) was cooled to 0° C. To this suspension was added a solution of [3[(1R)-3-(3,4-dimethoxyphenyl)-1-hydroxyphenyl]phenoxy]-acetic acid (6, 8.05 g, 23.24 mmol; compounds 6 and 14 may be prepared as provided in the present application) in THF (60 mL, 7.5 vol.) while maintaining the internal temperature below 20° C. The reaction was stirred for a further 2 hours at room temperature. [IPC TLC (in process check: thin layer chromatography) (SiO₂: EtOAc/Hexane 50:50) 6: Rf 0.15, 19: Rf 0.26]. Upon completion, the reaction mixture was cooled to 0° C. and water (1.5 mL) was added dropwise followed by 15% NaOH aq. (1.5 mL) and water (3 mL) while maintaining the internal temperature below 30° C. The quenched reaction was then diluted with TBME (tert-Butyl methyl ether) (100 mL, 12.0 vol.) and stirring continued for 30 minutes. The reaction mixture was dried with MgSO₄, filtered and solvent was removed by rotary evaporation. The crude product 3-(3,4-dimethoxyphenyl)-1-[3-(2-hydroxyethoxy)phenyl]propan-1-one 19 (7.10 g, 21.38 mmol, 92% yield) was obtained as a colorless oil and was used directly in the next step without further purification.

3-(3,4-dimethoxyphenyl)-1-[3-(2-hydroxyethoxy)phenyl]propan-1-one (19, 68.6 g, 206.5 mmol) was dissolved in DCM (Dichloromethane) (590 mL, 8.5 vol.). Triethylamine (43.2 mL, 309.8 mmol, 1.5 eq.) was added followed by 4-toluenesulfonyl chloride (39.4 g, 206.5 mmol, 1.0 eq.). The reaction mixture was stirred at room temperature for 18 hours. [IPC TLC (SiO₂: EtOAc/Hexane 50:50) 19: Rf 0.26, 20-OTs: Rf 0.52, TsCl: Rf 0.76]. Upon completion, the DCM was removed by rotary evaporation and exchanged for EtOAc (500 mL, 7.1 vol.). The organic layer was washed with HCl aq. 1M (3×100 mL, 1.4 vol.), NaHCO₃ sat. aq. (2×10 mL, 1.4 vol.), brine (70 mL, 1.0 vol.), dried over MgSO₄ then filtered and solvent was removed by rotary evaporation. The crude residue was purified by BIOTAGE (340 g SNAP Ultra column; 30-90% EtOAc in Hexane) to provide 2-{3-[3-(3,4-dimethoxyphenyl)propanoyl]phenoxy}ethyl 4-methylbenzenesulfonate, 20-OTs (63.6 g, 130.7 mmol, 63% yield) as a colorless oil. This material was used directly in the following step.

2-{3-[3-(3,4-dimethoxyphenyl)propanoyl]phenoxy}ethyl 4-methylbenzenesulfonate 20-OTs (3.93 g, 8.08 mmol) and N,N′-dimethyl-ethylenediamine (0.36 g, 4.04 mmol, 0.5 eq.) were dissolved in MeCN (acetonitrile) (20 mL, 5.0 vol.). To this solution was added K₂CO₃ (2.23 g, 16.16 mmol, 2.0 eq.) and KI (0.67 g, 4.04 mmol, 0.5 eq.). The reaction mixture was heated to 70° C. and stirred for 16 hours. [IPC TLC (SiO₂: EtOAc/Hexane 50:50) 20-OTs: Rf 0.26, 13-(CH₂)₂—: Rf baseline]. Upon completion, MeCN was removed by rotary evaporation and exchanged for EtOAc (50 mL, 12.0 vol.). The organic layer was washed with water (2×20 mL, 5.0 vol.) and NaHCO₃ sat. aq. (2×20 mL, 5.0 vol.). The desired product was then extracted into the aqueous layer with HCl 1M aq. (2×30 mL, 7.5 vol.) and the aqueous phase was washed with EtOAc (2×20 mL, 5.0 vol.). The aqueous phase was then basified with NaHCO₃ sat. aq. (90 mL, 22.5 vol.) and extracted with EtOAc (3×30 mL, 7.5 vol.). The organic phase was then washed with brine (20 mL, 5.0 vol.), dried over MgSO₄, filtered and solvent was removed by rotary evaporation to provide 13-(CH₂)₂— (2.75 g, 3.83 mmol, 95% yield). The crude material was used directly in the next step without further purification.

13-(CH₂)₂— (2.47 g, 3.45 mmol) and 14 (2.77 g, 7.59 mmol, 2.2 eq.) were dissolved in DCM (35 mL, 14.0 vol.) and the solution cooled to −15° C. To the reaction mixture was added EDCl (1-Ethyl-3-(3-dimethylaminopropyl)carbodiimide) (1.59 g, 8.28 mmol, 2.4 eq.) followed by DMAP (4-Dimethylaminopyridine) (1.86 g, 15.18 mmol, 4.4 eq.). The reaction was stirred overnight at −15° C. After 24 hours the internal temperature of the reaction was increased to −4° C. and the reaction left to stir for a further 24 hours. [IPC TLC (SiO₂: MeOH/DCM 5:95) 15-(CH₂)₂—: Rf 0.15, 14: Rf 0.37, Compound A: Rf 0.66]. Upon completion, the reaction was quenched by addition of 5M HCl sol. (10 mL, 4.0 vol.) to the reaction mixture at −4° C. DCM was then removed by vacuum distillation while maintaining the internal temperature below 20° C. The residue was diluted with EtOAc (50 mL, 20.0 vol.) and the organic layer washed sequentially with 1M HCl sol. (2×10 mL, 4.0 vol.), NaHCO₃ sol. (10 mL, 4.0 vol.) and brine (10 mL, 4.0 vol.). The organic layer was dried with MgSO₄, filtered and solvent removed by rotary evaporation. The crude residue was purified by BIOTAGE (50 g SNAP Ultra, 0-10% MeOH in DCM) to provide Compound A (2.09 g, 1.48 mmol, 43% yield) as an amorphous white solid. MS: (+ESI): calculated for C₈₀H₁₀₆N₄O₁₈ [M+H]⁺ 1411.7575, found 1411.7982.

### Example 2: Alternative Synthesis of Compound A

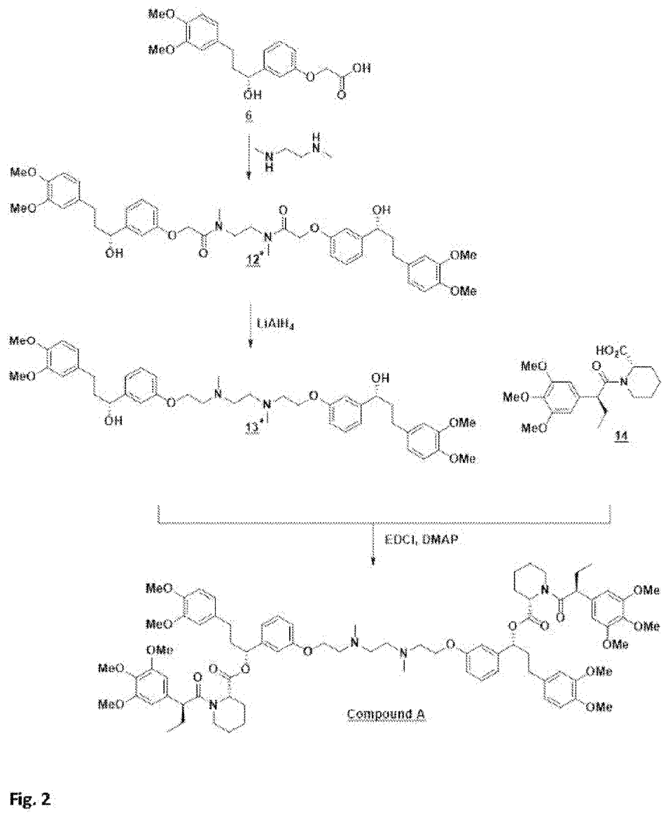

Compound A was synthesized according to the scheme shown in FIG. 2. Although the general synthetic scheme of FIG. 1 provides enhanced yields with less self-esterification, or intramolecular esterification of 6, both procedures are appropriate to obtain Compound A. Compound AP611 is used as the starting point for synthesis of Compound A. Numeric labels used for the intermediate compounds are consistent with those used in Methods 1-3 provided herein, however, where there are any differences, the indicated structure is the one shown in the drawings or synthetic schemes. In FIG. 1, 20* refers to 20, where the GAct² group is OTs. 12* and 13* refer to 12 and 13, respectively, where R¹ and R² are methyl, and R_{L} is —CH₂—CH₂—).

### Example 3: Preparation of Intermediate Compounds and Compound A

The present Example provides additional information regarding methods that may be used to synthesize Compound A, and certain intermediate compounds that may be used in the synthesis scheme.

### Synthetic Routes to Common Intermediates 6, 9 and 11 (Scheme 1):

The synthesis of 6 involves five chemical conversions and one chiral upgrade (see Method 1, herein); Claisen-Schmidt condensation between 1 and 2, reduction of the 3 double bond by transfer hydrogenation, alkylation of the 4 intermediate followed by asymmetric reduction of 5 and hydrolysis provides crude 6. Diasteromeric salt formation of crude 6 with (−)-cinchonidine followed by recrystallization and acidification yields 6 with chemical purity >98% and chiral purity >99.8%.

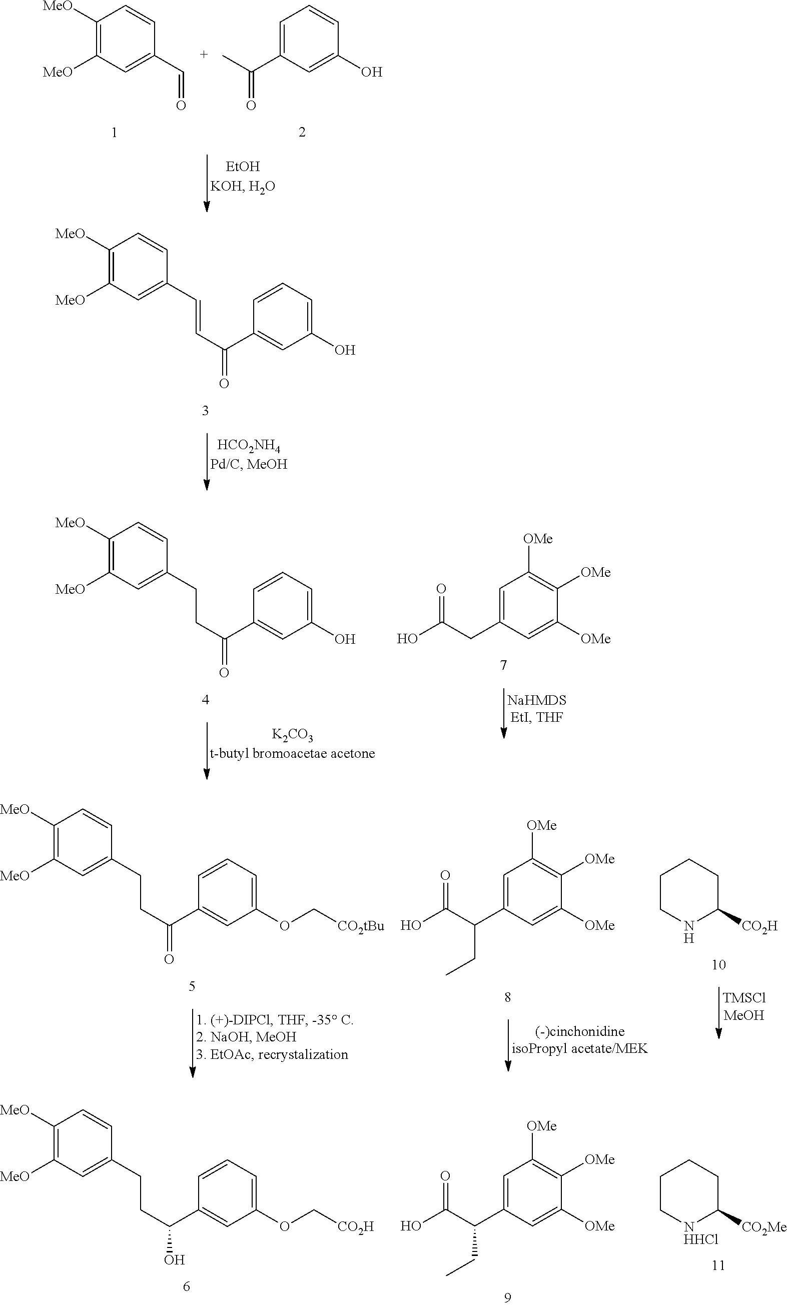

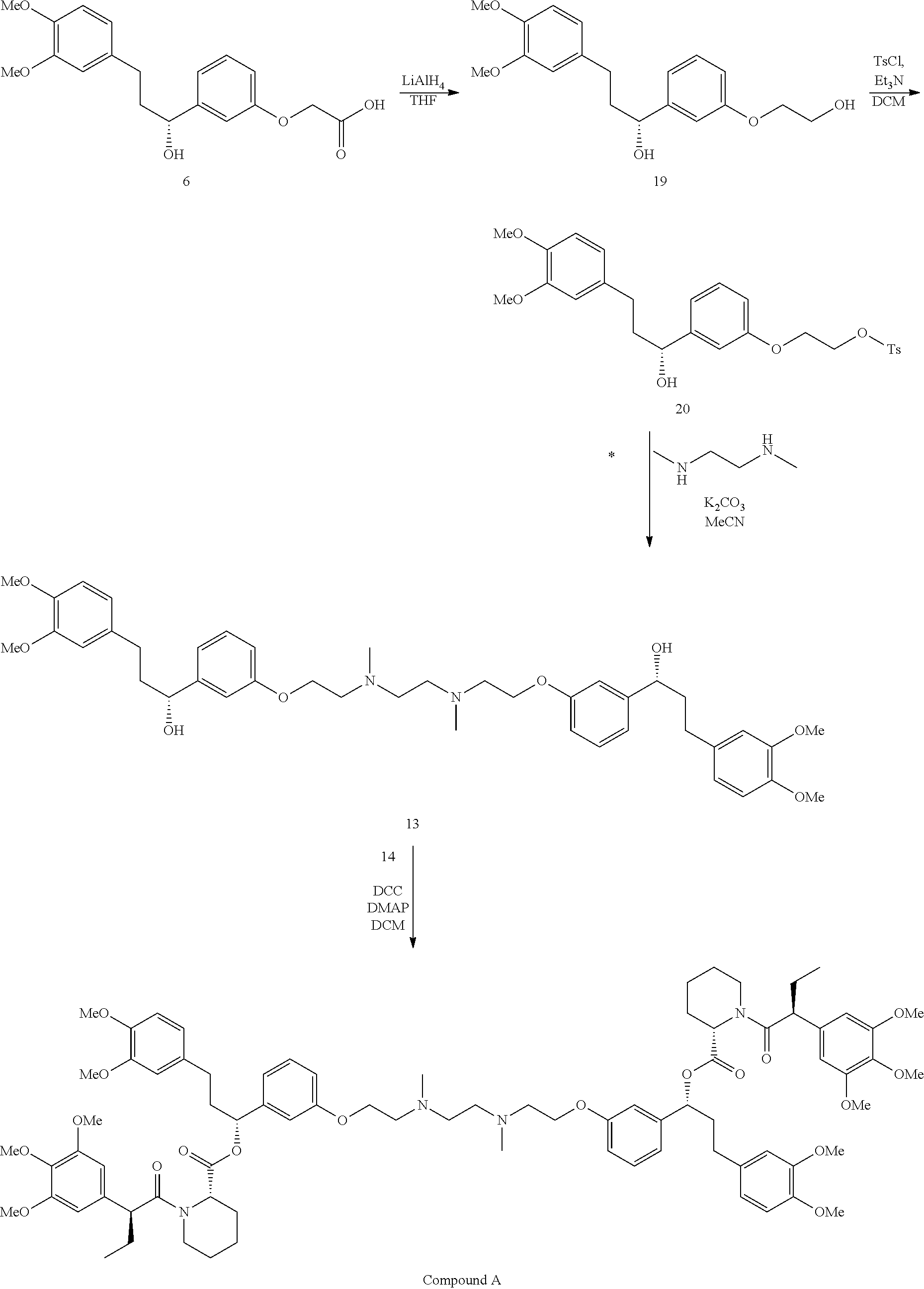

### Method:

3,4-Dimethoxybenzaldehyde (1, 39.9 g, 0.24 mol) and 3′-hydroxyacetophenone (2, 32.7 g, 0.24 mol, 1.0 eq.) were dissolved in EtOH (320 mL, 8.0 vol.) and the solution was cooled to 10° C. A solution of KOH (53.9 g, 0.96 mol, 4.0 eq.) in water (200 mL, 5.1 vol.) was added slowly to the reaction mixture while maintaining the internal temperature below 25° C. The reaction mixture was left to stir for 16 hours at 20° C. [IPC TLC (SiO₂: EtOAc/Hexane 50:50) 1: Rf 0.55, 2: Rf 0.45, 3: Rf 0.33.] Upon completion, the reaction was cooled to 0° C. and 37% HCl solution (102 mL, 2.6 vol.) was slowly added until pH 3 while maintaining the internal temperature below 25° C. DI water (250 mL, 6.3 vol.) was added and the resulting slurry stirred for 3 hours at 20° C. The product was filtered, washed with water (3×80 mL), and dried under vacuum at 40° C. for 72 hours to constant mass. The product 3,4-dimethoxy-3′-hydroxychalone 3 (60.0 g, 0.21 mol, 88% yield) was obtained as a yellow powder.

HPLC (High-performance liquid chromatography) analysis shows 3 (9.54 min) exhibits a weak negative absorption at 250-280 nm. Residual 1 (4.03 min) and 2 (4.58 min) were occasionally observed but could be effectively purged in the following step.

3,4-dimethoxy-3′-hydroxychalone (3, 42.8 g, 150.5 mmol) and 10% Pd/C 50% w/w wet (4.2 g, 10 wt %, 0.1 vol.) were suspended in MeOH (213 mL, 5.0 vol.). NH₄OCHO (14.2 g, 225.8 mmol, 1.50 eq.) was added in one portion. The slurry was heated to 55° C. for 1.5 hours. [IPC TLC (SiO₂: EtOAc/Hexane 50:50) 3: Rf 0.41, 4: Rf 0.52.]. Upon completion the reaction was cooled to 45° C. and filtered, while hot, through Celite and rinsed forward with MeOH (60 mL, 1.5 vol.). The combined filtrate was reheated to 50° C. to ensure a clear solution. DI water (291 mL, 6.8 vol.) was added over 1 hour while keeping internal temperature at 50° C. [NOTE: Significant precipitation had occurred after 50% of the addition]. The resulting slurry was cooled to 20° C. over a period of 3 hours and left to stir for a further 12 hours. The product was filtered, washed with DI water (2×80 mL, 2.0 vol.) and dried under vacuum at 40° C. for 48 hours to constant mass. The product 3-(3,4-dimethyoxyphenyl)-1-(3-hydroxyphenyl)-1-propanone 4 (36.6 g, 0.13 mol, 85% yield) was obtained as a colorless powder.

HPLC analysis showed 4 (6.87 min) and two minor impurities <1% by area.

3-(3,4-dimethyoxyphenyl)-1-(3-hydroxyphenyl)-1-propanone (4, 40.1 g, 0.14 mol) and K₂CO₃ powder (29.0 g, 210.0 mmol, 1.50 eq.) were suspended in acetone (400 mL, 10.0 vol.) at 20° C. To the reaction mixture was added tert-butyl bromoacetate (27.9 mL, 189 mmol, 1.35 eq.) over 10 minutes. The resulting suspension was heated to 55° C. for 12 hours. [IPC TLC (SiO₂: EtOAc/Hexane 50:50) 4: Rf 0.57, 5: Rf 0.73]. Upon completion, the reaction was cooled to 20° C., filtered and the washed forward with acetone (20 mL, 0.5 vol.). The acetone was removed by rotary evaporation at 40° C. and co-evaporated with TBME (2×200 mL, 5.0 vol.). The residue was dissolved in TBME (88 mL, 2.2 vol.) and filtered through a silica plug (10 g, 0.3 vol.) and washed forward with TBME (56 mL, 1.4 vol.). The combined filtrate was stirred for 12 hours at 20° C. then cooled further to 0° C. for a further 2 hours prior to filtration. The filter cake was then washed with 20% TBME in Heptane (40 mL, 1.0 vol.) and dried under vacuum at 20° C. for 24 hours to constant mass. The product 3[3-(3,4-dimethyoxyphenyl)-1-oxopropyl]phenoxy]-acetic acid, 1,1-dimethylester 5 (39.8 g, 99.4 mmol, 71% yield) was obtained as a colorless crystalline powder.

HPLC analysis showed crystalline 5 (6.72 min) to be excellent purity with no detectable impurities.

3[3-(3,4-dimethyoxyphenyl)-1-oxopropyl]phenoxy]-acetic acid, 1,1-dimethylester (5, 31.60 g, 78.91 mmol) was dissolved in THF (170 mL, 5.4 vol.) and cooled to −40° C. A solution of (+)-DIPCI [62.5 wt % in Hexanes] (121.50 g, 140.8 mL, 236.74 mmol, 3.0 eq.) was added over 15 minutes ensuring the reaction remain below −30° C. After complete addition the reaction was stirred for a further 1 hour, then allowed to warm to −15° C. over 1 hour, then to room temperature over a further 1 hour. [IPC TLC (SiO₂: EtOAc/Hexane 50:50) 5: Rf 0.65, Intermediate ester: Rf 0.49, 6: Rf 0.15]. Upon completion the reaction was cooled to 0° C. and water (63 mL, 2.0 vol.) added slowly while maintaining the internal temperature below 20° C. The mixture was stirred for 15 minutes, separated and the organic layer evaporated to dryness. The resulting oil was dissolved in MeOH (63 mL, 2.0 vol.), cooled to 10° C. and 5M NaOH aq. (76 mL, 2.4 vol.) added slowly while maintaining the temperature below 25° C. The reaction was stirred vigorously for 30 minutes at 20° C. until complete hydrolysis to the acid was observed by TLC. The reaction was concentrated to remove MeOH. 5M HCl aq. (54 mL, 1.7 vol.) was added to the oil to pH 7. After 30 minutes of agitation the aqueous layer was then washed with EtOAc (2×60 mL, 1.8 vol.). 5M HCl aq. (16 mL, 0.5 vol.) was then added to the aqueous solution to pH 2. The mixture was diluted with water (63 mL, 2.0 vol.) and heptane (22 mL, 0.7 vol.). The mixture was agitated for 16 hours at 20° C. The resulting slurry was filtered and washed with water (63 mL, 2.0 vol.) and the crude product dried under vacuum at 40° C. for 24 hours to constant mass. The product [3[(1R)-3-(3,4-dimethoxyphenyl)-1-hydroxyphenyl]phenoxy]-acetic acid 6 (16.95 g, 48.93 mmol, 62% yield, 97% e.e.) was obtained as a lumpy white solid.

HPLC analysis shows desired (R)-6 (10.98 min) and undesired (S)-6 (9.73 min).

### Alternate Synthetic Route to Intermediate 6

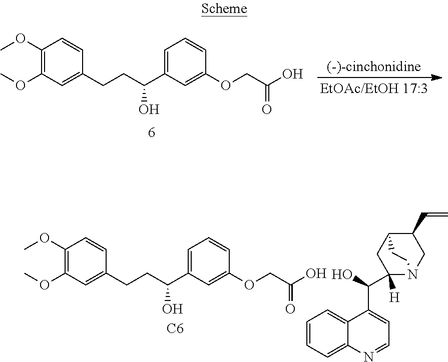

### Scheme 8.

To improve chiral purity of intermediate 6, the product was converted into a diastereomeric salt, in order to separate the two diastereomers. Following enrichment of the R salt, the salt is converted to an enriched product 6, with about 99.5% chiral purity. An initial attempt at the salt formation and recrystallization was based on patent (PCT Patent Application No: PCT/US2012/022642, filed Jan. 26, 2012, PCT Patent Application Publication No: WO2012103279A2, published Aug. 2, 2012, Li, F., et al., “Methods and compositions for the synthesis of multimerizing agents”, which is hereby incorporated by reference herein in its entirety) and provided a 37% isolated yield. Subsequent efforts to combine the initial salt formation with the crystallization gave >80% yields, however <99.6% chiral purity was achieved. By adjusting the volumes of isoproponal used during the scale-up, a thick slurry was formed, which was effectively removed from the vessel by re-charging the alcoholic filtrate. Eventually, the isolated product was obtained in the expected yield.

### Protocol

[3[(1R)-3-(3,4-dimethoxyphenyl)-1-hydroxyphenyl]phenoxy]-acetic acid (6, 210 g, 0.60 mol) and (−)-cinchonidine (178 g, 0.60 mol, 1.0 eq.) were dissolved in EtOAc (5.2 L, 25.0 vol.) and heated to 50° C. for 1 hour. The reaction mixture was cooled to 20° C. and agitated for a further 16 hours. The product was isolated by filtration and washed with EtOAc (0.7 L, 3.5 vol.). The product was dried under vacuum at 40° C. for 16 hours to constant mass. The product C6 (383 g, 0.60 mol, 99% yield) was obtained as a white powder.

C6 (cinchonidine salt) (383 g, 0.60 mol) was re-dissolved in iPrOH (3.5 L, 9.0 vol. relative to C6) and heated to 50° C. Once fully dissolved the solution was allowed to cool to 20° C. over 3 hours and stirred for a further 12 hours at 20° C. The solid was filtered and washed with EtOAc (0.8 L, 2.0 vol.) then dried under vacuum at 40° C. to constant mass for 16 hours. The product C6 (345 g, 0.54 mol, 89% yield over 2 steps) was obtained as a colorless powder.

### Analysis

A small sample was subject to acidification, extraction and isolation to provide an IPC sample prior to acidification of the bulk material. HPLC analysis revealed a 99.8% chiral purity, in line with specification for the GMP starting material that would be used for the manufacture of Compound A.

HPLC analysis showed desired (R)-6 (10.67 min) and undesired (S)-6 (9.46 min).

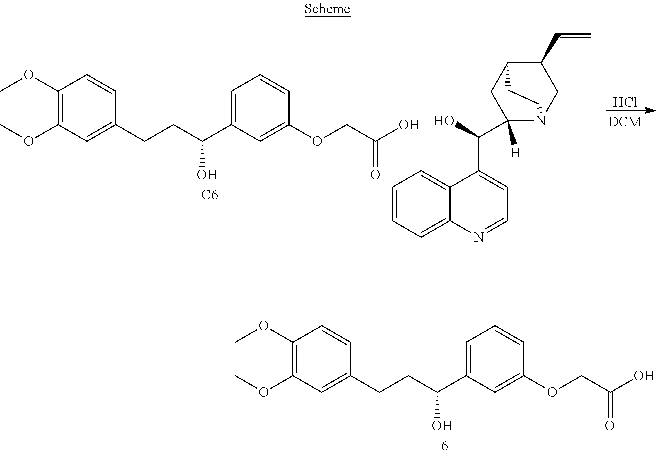

An alternative method improve chiral purity of the R form of 6 involved acidification of C6 followed by extraction to provide 6. Post extraction and solvent switch from dichloromethane (DCM) into isopropanol, the solution was charged into water leading to the immediate precipitation of the product. After stirring overnight, the mobile product slurry was easily filtered and isolated in the expected yield.

### Protocol

[3[(1R)-3-(3,4-dimethoxyphenyl)-1-hydroxyphenyl]phenoxy]-acetic acid (−)-cinchonidine salt (C6, 6.49 g, 10.12 mmol) was dissolved in DCM (20 mL, 3.0 vol.) and water (26 mL, 4.0 vol.) at 20° C. 5M HCl aq. (10 mL, 1.6 vol.) was then added and the biphasic mixture stirred for 1 hour at 20° C. The organic layer was separated and washed with 1M HCl aq. (2×7 mL, 1.0 vol.) and brine (7 mL, 1.0 vol.). The DCM was removed by rotary evaporation and then co-distilled with iPrOH (2×20 mL, 3.0 vol.) to 7 mL total volume. The thick iPrOH solution was added slowly to a separate vessel containing DI water (80 mL, 12.0 vol.), seeded with pure 6 and stirred for 16 hours. The product was filtered and washed with DI water (2×13 mL, 2.0 vol.) and then dried under vacuum at 40° C. to constant mass for 16 hours. The product [3[(1R)-3-(3,4-dimethoxyphenyl)-1-hydroxyphenyl]phenoxy]-acetic acid 6 (2.89 g, 8.36 mmol, 83% yield, 99.5% e.e.) was obtained as a lumpy white powder. [NOTE: The chiral purity is not degraded once 6 has been subject to chiral upgrade.]

### Analysis

HPLC analysis showed desired (R)-6 (11.06 min) and undesired (S)-6 (9.84 min).

### Synthetic Route to Common Intermediate 14 (Scheme 2):

The synthesis of GMP (Good Manufacturing Practice) intermediate 14 involves two chemical transformations and one purification (Scheme 2); Amide coupling between 9 and 11 followed by hydrolysis of the methyl ester provides crude 14. Recrystallization of crude 14 from ethanol yields 14 with chemical purity >99.0% and chiral purity >99.5%.

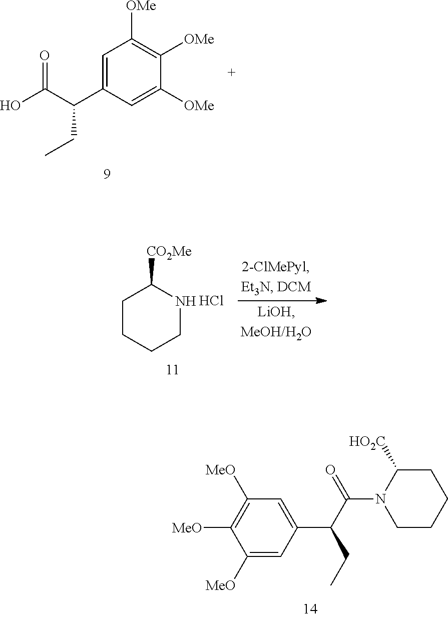 [SMILES: CC[C@H](C(=O)O)c1cc(OC)c(OC)c(OC)c1 ; COC(=O)[C@@H]1CCCCN1 ; Cl ; CC[C@H](C(=O)N1CCCC[C@H]1C(=O)O)c1cc(OC)c(OC)c(OC)c1]

### Method:

(2S)-2-(3,4,5-Trimethoxyphenyl)butanoic acid 9 (93.0 g, 0.37 mol), Methyl (2S)-piperidinecarboxylate hydrochloride 11 (69.0 g, 0.38 mol, 1.1 eq.) and 2-chloro-1-methylpyridinium iodide (117.0 g, 0.46 mol, 1.3 eq.) were dissolved in DCM (720 mL, 8.0 vol.) and cooled to 12° C. To the reaction mixture was added a solution of triethylamine (111.0 g, 1.10 mol, 3.0 eq.) in DCM (180 mL, 2.0 vol.) while maintaining the internal temperature below 25° C. The reaction was then allowed to stir for 3-4 hours at 22° C. [IPC HPLC (C18: ACN/H₂O 10-90%, 20 min, 1 mL/min) 9: rrt 0.53 min, Intermediate ester: rrt 0.69, 14: rrt 0.58]. Upon completion, the DCM was removed by rotary evaporation and exchanged for MeOH (225 mL, 2.5 vol.). To the methanolic solution was added a solution of LiOH.H₂O (77.0 g, 1.83 mol, 5.0 eq.) in DI water (225 mL, 2.5 vol.) and the reaction was stirred for 12 hours at 22° C. Upon completion, as determined by IPC, additional water (180 mL, 2.0 vol.) was added and the solution is vacuum distilled to remove methanol. EtOAc (450 mL, 5.0 vol.) was added and the biphasic solution stirred vigorously for 10 minutes after which the phases were separated. The aqueous layer containing the product was removed and the organic phase was washed again with water (180 mL, 2.0 vol.) and 10% NaHCO₃ sol. (360 mL, 4.0 vol.). The combined aqueous layers were cooled to 4° C. A solution of 5M HCl was slowly added until the pH 4. The resulting slurry was stirred at 4° C. for 3 hours then the solid was collected by vacuum filtration. The crude 14 (117.0 g, 0.32 mol) was dissolved in EtOH (1080 mL, 12.0 vol.), heated and maintained at 50° C. for 30 minutes. The ethanolic solution was then cooled to 0° C. over 6 hours. The resulting crystalline solid is collected by vacuum filtration and washed with cold EtOH (90 mL, 1.0 vol.). The product 14 (87.5 g, 0.24 mol, 66% yield) was obtained a colorless crystalline solid.

Reverse phase HPLC analysis showed crystalline 14 (11.64 min) to be excellent purity with no detectable impurities. Chiral HPLC analysis shows crystalline 14 (32.58 min) to be excellent purity with no detectable chiral impurities.

### Synthetic Route to Compound A (Scheme 3):

The synthesis of Compound A involves two further transformations of the 6 fragment prior to dimerization and coupling with 14 (Scheme 3); Reduction of 6 and tosylation of the resulting alcohol 6-OH followed by dimerization with dimethyl diaminoethylene to provide 13* (where R¹ and R² are methyl, R_{L} is —CH₂—CH₂—). Extraction of 13* into the aqueous phase provides a strategy for purification. Finally, amide coupling between 13* and 14 yields Compound A which is then subject to chromatographic purification.

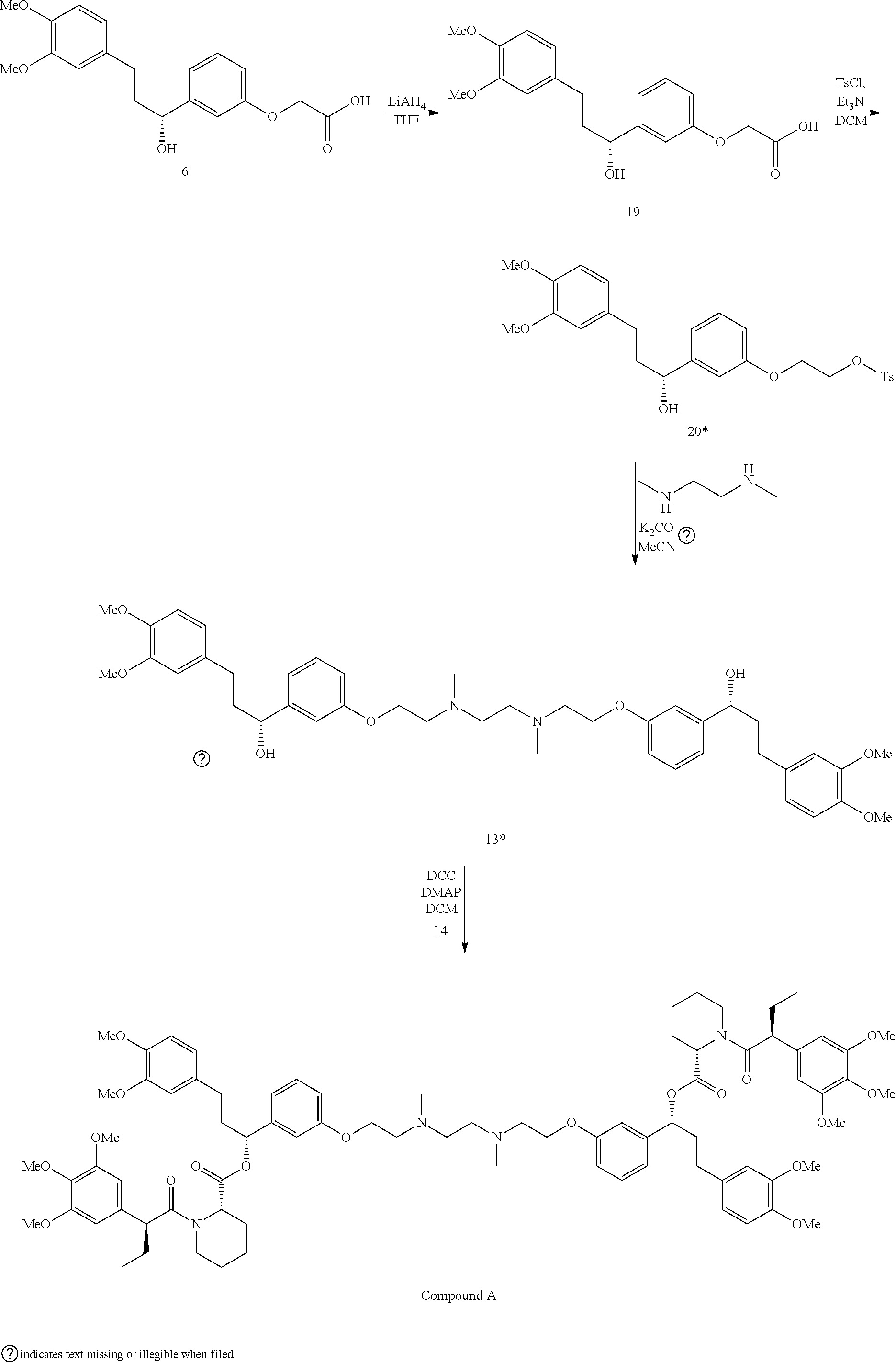

### Method:

A suspension of LiAlH₄ (1.41 g, 37.18 mmol, 0.2 vol.) in THF (74 mL, 9.0 vol.) was cooled to 0° C. To this suspension was added a solution of [3[(1R)-3-(3,4-dimethoxyphenyl)-1-hydroxyphenyl]phenoxy]-acetic acid (6, 8.05 g, 23.24 mmol) in THF (60 mL, 7.5 vol.) while maintaining the internal temperature below 20° C. The reaction was stirred for a further 2 hours at room temperature. [IPC TLC (SiO₂: EtOAc/Hexane 50:50) 6: Rf 0.15, 19: Rf 0.26]. Upon completion, the reaction mixture was cooled to 0° C. and water (1.5 mL) was added dropwise followed by 15% NaOH aq. (1.5 mL) and water (3 mL) while maintaining the internal temperature below 30° C. The quenched reaction was then diluted with TBME (100 mL, 12.0 vol.) and stirring continued for 30 minutes. The reaction mixture was dried with MgSO₄, filtered and solvent removed by rotary evaporation. The crude product 3-(3,4-dimethoxyphenyl)-1-[3-(2-hydroxyethoxy)phenyl]propan-1-one 19 (7.10 g, 21.38 mmol, 92% yield) was obtained as a colorless oil and was used directly in the next step without further purification.

Reverse phase HPLC analysis showed crude 19 (8.71 min) to be excellent purity with no major impurities. The material is carried through directly into the following step.

3-(3,4-dimethoxyphenyl)-1-[3-(2-hydroxyethoxy)phenyl]propan-1-one (19, 68.6 g, 206.5 mmol) was dissolved in DCM (590 mL, 8.5 vol.). Triethylamine (43.2 mL, 309.8 mmol, 1.5 eq.) was added followed by 4-toluenesulfonyl chloride (39.4 g, 206.5 mmol, 1.0 eq.). The reaction mixture was stirred at room temperature for 18 hours. [IPC TLC (SiO₂: EtOAc/Hexane 50:50) 19: Rf 0.26, 20: Rf 0.52, TsCl: Rf 0.76]. Upon completion, the DCM was removed by rotary evaporation and exchanged for EtOAc (500 mL, 7.1 vol.). The organic layer was washed with HCl aq. 1M (3×100 mL, 1.4 vol.), NaHCO₃ sat. aq. (2×10 mL, 1.4 vol.), brine (70 mL, 1.0 vol.), dried over MgSO₄ then filtered and solvent removed by rotary evaporation. The crude residue was purified by Biotage (340 g SNAP Ultra column; 30-90% EtOAc in Hexane) to provide 2-{3-[3-(3,4-dimethoxyphenyl)propanoyl]phenoxy}ethyl 4-methylbenzenesulfonate, 20 (63.6 g, 130.7 mmol, 63% yield) as a colorless oil. This material was used directly in the following step.

Reverse phase HPLC analysis showed crude 20 (24.36 min) to be 88% purity with several small impurities present even after chromatographic purification.

2-{3-[3-(3,4-dimethoxyphenyl)propanoyl]phenoxy}ethyl 4-methylbenzenesulfonate 20 (3.93 g, 8.08 mmol) and N,N′-dimethyl diaminoethylene (0.36 g, 4.04 mmol, 0.5 eq.) were dissolved in MeCN (20 mL, 5.0 vol.). To this solution were added K₂CO₃ (2.23 g, 16.16 mmol, 2.0 eq.) and KI (0.67 g, 4.04 mmol, 0.5 eq.). The reaction mixture was heated to 70° C. and stirred for 16 hours. [IPC TLC (SiO₂: EtOAc/Hexane 50:50) 20: Rf 0.26, 13* Rf baseline]. 13* refers to compound 13 of method 3, where R_{L} is —CH₂—CH₂—. Upon completion, MeCN was removed by rotary evaporation and exchanged for EtOAc (50 mL, 12.0 vol.). The organic layer was washed with water (2×20 mL, 5.0 vol.) and NaHCO₃ sat. aq. (2×20 mL, 5.0 vol.). The desired product was then extracted into the aqueous layer with HCl 1M aq. (2×30 mL, 7.5 vol.) and the aqueous phase was washed with EtOAc (2×20 mL, 5.0 vol.). The aqueous phase was then basified with NaHCO₃ sat. aq. (90 mL, 22.5 vol.) and extracted with EtOAc (3×30 mL, 7.5 vol.). The organic phase was then washed with brine (20 mL, 5.0 vol.), dried over MgSO₄, filtered and solvent removed by rotary evaporation to provide 13* (2.75 g, 3.83 mmol, 95% yield). The crude material was used directly in the next step without further purification.

Reverse phase HPLC analysis shows crude 13* (15.62 min) to be 79% purity with a lot of minor impurities and two significant impurities around 5% (9.72 min and 20.16 min). This crude material was used directly in the next step.

13* (2.47 g, 3.45 mmol) and 14 (2.77 g, 7.59 mmol, 2.2 eq.) were dissolved in DCM (35 mL, 14.0 vol.) and the solution cooled to −15° C. To the reaction mixture was added EDCl (1.59 g, 8.28 mmol, 2.4 eq.) followed by DMAP (1.86 g, 15.18 mmol, 4.4 eq.). The reaction was stirred overnight at −15° C. After 24 hours the internal temperature of the reaction was increased to −4° C. and the reaction left to stir for a further 24 hours. [IPC TLC (SiO₂: MeOH/DCM 5:95) 13*: Rf 0.15, 14: Rf 0.37, Compound A: Rf 0.66]. Compound A may also be referred to as 15*, which indicates 15 as provided in Method 3, where R¹ and R² are methyl, R_{L} is —CH₂—CH₂—. Upon completion, the reaction was quenched by addition of 5M HCl sol. (10 mL, 4.0 vol.) to the reaction mixture at −4° C. DCM was then removed by vacuum distillation while maintaining the internal temperature below 20° C. The residue was diluted with EtOAc (50 mL, 20.0 vol.) and the organic layer washed sequentially with 1M HCl sol. (2×10 mL, 4.0 vol.), NaHCO₃ sol. (10 mL, 4.0 vol.) and brine (10 mL, 4.0 vol.). The organic layer was dried with MgSO₄, filtered and solvent removed by rotary evaporation. The crude residue was purified by Biotage (50 g SNAP Ultra, 0-10% MeOH in DCM) to provide Compound A (2.09 g, 1.48 mmol, 43% yield) as an amorphous white solid. DCC denotes N,N′-Dicyclohexylcarbodiimide

Reverse phase HPLC analysis shows Compound A (29.06 min) after one chromatographic purification. The major impurity (28.02 min) making up 8% corresponds to the mono-coupled intermediate. Further HPLC and LCMS method development will be needed to establish the diastereomeric purity.

### Example 4: Characterization of Compound A

Compound A, prepared according to the synthetic scheme of FIG. 1 was synthesized, with a diastereomeric purity of 92%. The phosphate salt (Compound A.1) and the hydrochloride salt (Compound A.2), in the form of amorphous powders are discussed herein, however other counter-ions may be used to produce the appropriate salts. Initial assays indicate that the phosphate salt has greater aqueous solubility than the hydrochloride salt of Compound A, and both salts have greater solubility than rimiducid.

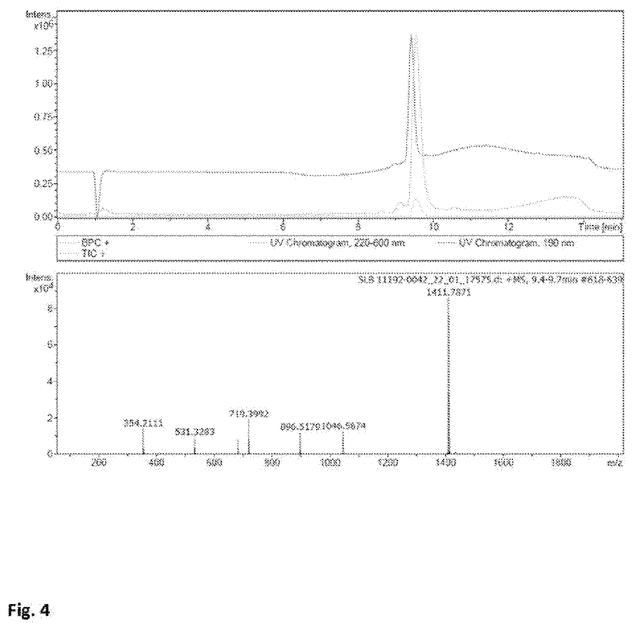

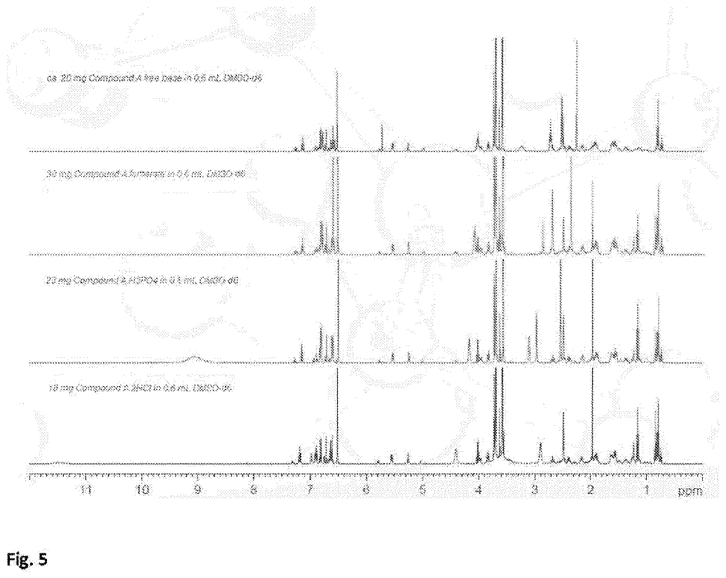

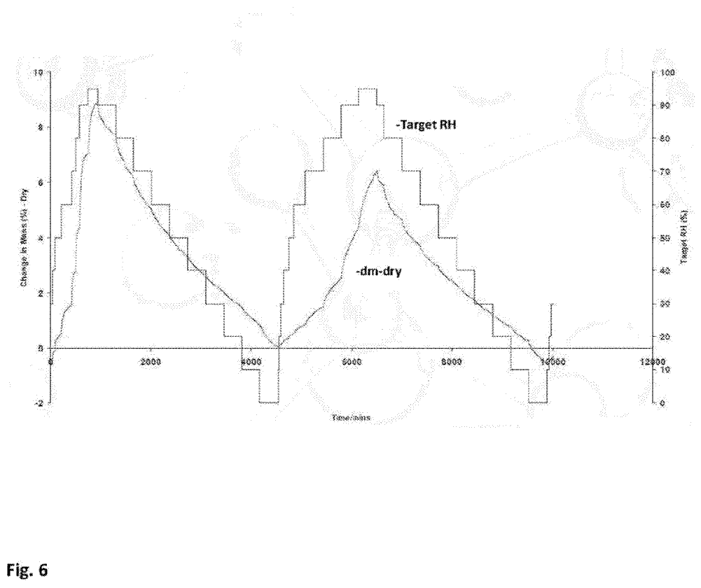

FIG. 4 provides structural LCM verification of Compound A.1, with a molecular weight of 1412, corresponding to the formula weight of the compound. FIG. 5 provides proton NMR verification of Compound A, Compound A.1, and Compound A.2. Amorphous Compound A.1 is soluble in water; approximately 2.5 mg of the amorphous powder was soluble in 1 mL of water. A dynamic vapor sorption isotherm showed the equilibrium amount of vapor sorbed as a function of steady state relative vapor pressure at a constant temperature of 25° C. indicated an increase of equilibrium weight of approximately 6 to 7% when exposed to a relative humidity of approximately 80% (FIG. 6).

The equilibrium solubilities of Compound A.1 in phosphate, acetate, and citrate-phosphate buffer, as well as unbuffered water are shown in Table 2. The compound appears to be soluble in unbuffered water (shown as “none” below) or in acetate buffers of pH 4.0 or less.

TABLE 2

| | | Solubility/mg · mL⁻¹ | | | | |
|---|---|---|---|---|---|---|
| Buffer | | | | | | Benzene |
| system | pH | Hydrochloride | Sulphate | Phosphate | Fumarate | sulphonate |
| None | Inherent pH | 1.06 (2.9) | 0.02 (3.06) | 2.77 (3.9) | 0.23 (3.4) | 0.06 (4.3) |
| | in parenthesis | | | | | |
| Phosphate | 5 | 0.035 | 0.028 | 0.016 | 0.017 | 0.006 |
| | 6 | 0.002 | ND | 0.001 | ND | ND |
| | 7 | ND | ND | 0.001 | ND | ND |
| Acetate | 4 | 0.239 | 0.050 | 4.611 | 0.069 | 0.054 |
| | 5 | 0.017 | 0.013 | 0.024 | 0.012 | 0.010 |
| Citrate- | 4 | — | — | 0.002 (1) | — | — |
| Phosphate | 4.5 | — | — | 0.001 (1) | — | — |
| | 5.0 | — | — | 0.000 (6) | — | — |
| | 5.5 | — | — | 0.000 (4) | — | — |

The stability of different salt forms of Compound A was assayed under accelerated conditions of heat and humidity for 7 and for 14 days. All salts were soluble up to 40° C. and 75% relative humidity (RH), but appeared to degrade under severe temperature and humidity conditions of 80° C. with or without 80% relative humidity. (Table 3)

TABLE 3

| | Assay (%) | | | | | | | |
|---|---|---|---|---|---|---|---|---|
| | 25° C./60% RH | | 40° C./75% RH | | 80° C. | | 80° C./80% RH | |
| Salt form | 7 D | 14 D | 7 D | 14 D | 7 D | 14 D | 7 D | 14 D |
| Hydrochloride | 97.3 | 96.7 | 97.2 | 96.9 | 96.3 | 94.8 | 95.6 | 94.4 |
| Sulphate | 96.2 | 95.0 | 95.9 | 95.6 | 95.3 | 94.6 | 88.2 | 85.4 |
| Phosphate | 96.2 | 95.9 | 95.6 | 95.9 | 94.9 | 93.3 | 85.2 | 77.7 |
| Fumarate | 97.5 | 97.1 | 97.2 | 96.8 | 96.6 | 95.8 | 87.5 | 81.2 |
| Benzenesulphonate | 92.2 | 91.7 | 92.2 | 92.2 | 91.5 | 90.6 | 91.2 | 90. |

### Example 5: Binding Activity of Multimeric Compounds

Cell Line Maintenance and Transfection:

Early passage HEK293T/16 cells (ATCC, Manassas, Va.) are maintained in IMDM, GlutaMAX™ (Life Technologies, Carlsbad, Calif.) supplemented with 10% FBS, 100 U/mL penicillin, and 100 U/mL streptomycin until transfection in a humidified, 37° C., 5% CO₂/95% air atmosphere. Cells in logarithmic-phase growth are transiently transfected with 800 ng to 2 μg of expression plasmid encoding inducible chimeric caspase-9 polypeptide and 500 ng of an expression plasmid encoding SRα promoter driven SEAP per million cells in 15-mL conical tubes. GeneJammer® Transfection Reagent at a ratio of 3 μl per ug of plasmid DNA is used to transiently transfect HEK293T/16 cells in the absence of antibiotics. 100 μl or 2 mL of the transfection mixture is added to each well in 96-well or 6-well plate, respectively. For SEAP assays, log dilutions of control rimiducid, multimeric compounds described herein, or pharmaceutically acceptable salts thereof, are added after a minimum 3-hour incubation post-transfection. For western blots, cells are incubated for 20 minutes with AP1903 (10 nM) before harvesting.

Assay Methods

The test compounds may be assayed for their ability to bind to FKBP12v36 and induce apoptosis in cells that express an inducible chimeric caspase-9 polypeptide. Apoptosis is measured as a reduction in secreted alkaline phosphatase (SEAP), expressed in the cells from a SEAP expression plasmid.

Twenty-four to forty-eight hours after treatment with the test compound, ˜100 μl of supernatants are harvested into a 96-well plate and assayed for SEAP activity as discussed, for example, in MacCorkle, R. A., K. W. Freeman, and D. M. Spencer, Proc Natl Acad Sci USA, 1998. 95(7): 3655-60, Spencer, D. M., et al., Science (1993) 262:1019-1024), and in Spencer, D. M., et al., Curr Biol, 1996. 6(7): p. 839-47. Briefly, after 65° C. heat denaturation for 45 minutes to reduce background caused by endogenous (and serum-derived) alkaline phosphatases that are sensitive to heat, 5 μl of supernatants are added to 95 μl of PBS and added to 100 μl of substrate buffer, containing 1 μl of 100 mM 4-methylumbelliferyl phosphate (4-MUP; Sigma, St. Louis, Mo.) re-suspended in 2 M diethanolamine. Hydrolysis of 4-MUP by SEAP produces a fluorescent substrate with excitation/emission (355/460 nm), which can be easily measured. Assays are performed in black opaque 96-well plates to minimize fluorescence leakage between wells. Following manufacturer's suggestions, 1 mL of IMDM+10% FBS without antibiotics is added to each mixture. 1000-μl of the mixture is seeded onto each well of a 96-well plate. 100-μl of AP1903 or multimeric compound described herein, or pharmaceutically acceptable salt thereof is added at least three hours post-transfection. After addition of the compound for at least 24 hours, 100-μl of supernatant is transferred to a 96-well plate and heat denatured at 68° C. for 30 minutes to inactivate endogenous alkaline phosphatases. For the assay, 4-methylumbelliferyl phosphate substrate is hydrolyzed by SEAP to 4-methylumbelliferon, a metabolite that can be excited with 364 nm and detected with an emission filter of 448 nm. Since SEAP is used as a marker for cell viability, reduced SEAP reading corresponds with increased iCaspase-9 activities, which corresponds to greater induction of the chimeric caspase-9 polypeptide following treatment with the test compound. Compounds that result in reduced SEAP readings correspond to greater binding of the compound to the multimerizing region.

Assays of Test Compounds

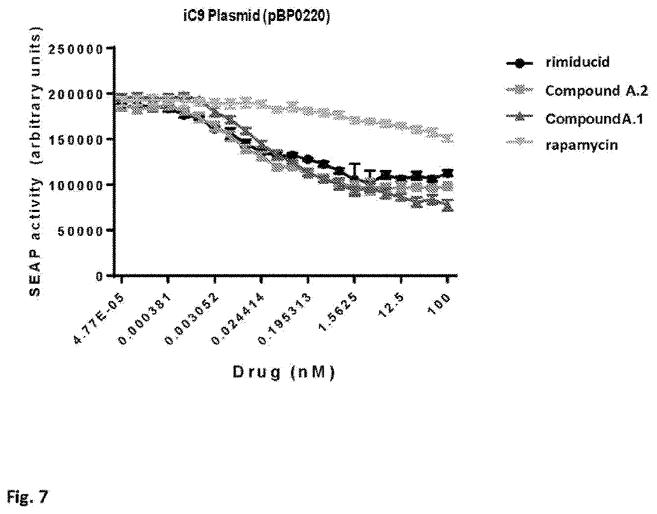

To examine binding activity of multimeric compounds, referred to as “test compound” in this Example, early-passage human embryonic kidney (HEK293T/16) cells were co-transfected with 500 ng of a SEAP expression plasmid), which is used as a marker for cell viability, and with (500 ng) of an expression vector that encodes an inducible Caspase-9 polypeptide (iC9), having a FKBP12v36 multimerizing region (pM101-pSFG-iC9 T2AΔCD19). Following manufacturer's suggestions, 1 mL of Iscove's Modified Dulbecco's Medium (IMDM, Thermo Fisher Scientific) +10% fetal bovine serum (FBS) without antibiotics was added to each mixture. 1000-ul of the mixture was seeded onto each well of a 96-well plate. The test compound, in the amounts indicated in FIG. 7, was added at least three hours post-transfection. After at least 24 hours, 100-ul of supernatant was transferred to a 96-well plate. The 96-well plates were wrapped to prevent evaporation and incubated at 68° C. for 30 minutes to inactivate endogenous and serum phosphatases while the heat-stable SeAP reporter remained. 100-μL media samples from each well were loaded into individual wells of a 96-well assay plate with black sides (Greiner). 100 μL of a solution of 1.0 M 4-methylumbelliferyl phosphate (4-MUP) in 1.0 M diethanolamine carbonate at pH 10.0 was added to each sample well to provide for a final concentration of 0.5 M for both. The samples were incubated for 4 to 16 hours. Hydrolysis of 4-MUP by SEAP produces a fluorescent product, which can be easily measured. Phosphatase activity was measured by fluorescence with excitation at 355 nm and emission at 460 nm. Data was transferred to a Microsoft Excel spreadsheet for tabulation and graphed with GraphPad Prism.

An initial assessment of binding activity of Compound A.1 and Compound A.2 to FKBP12-F36V was made using human embryonic kidney (293 HEK) cells in a multimerizing assay. Initially, 293 HEK cells were transiently co-transfected with secreted alkaline phosphatase reporter plasmid and an expression vector encoding an apoptotic switch containing a FKBP-F36V domain and a Caspase-9 polypeptide that mediates homodimerization and subsequent induction of programmed cell death in the presence of dimeric FKBP-F36V binding compounds. Compounds were first dissolved in ethanol and then cell cultures were exposed to the compounds of interest across a broad concentration range (4.77×10⁻⁵ to 100 nM). Here binding to FKBP12-F36V domain within the chimeric inducible caspase polypeptide by the conjugate ligand (e.g., rimiducid) leads to apoptosis with a resultant reduction in secreted alkaline phosphatase (reporter protein). Rapamycin, which does not homodimerize FKBP-F36V domain-containing proteins at low concentrations, may be used as a negative control. Under the experimental conditions, both Compound A.1 and Compound A.2 appear to be as active as rimiducid, with IC₅₀ values nearly identical to that of rimiducid, all ≤0.02 nM. (FIG. 7)

pM101--pSFG-iC9.T2A-ΔCD19

| Region | Nucleotide sequence | Amino acid sequence |
|---|---|---|
| FKBP12v36 | SEQ ID NO: 1 | SEQ ID NO: 2 |
| | ATGCTCGAGGGAGTGCAGGTGGAGACTATCTCCCC | MLEGVQVETISPGDGRTFP |
| | AGGAGACGGGCGCACCTTCCCCAAGCGCGGCCAGA | KRGQTCVVHYTGMLEDGK |
| | CCTGCGTGGTGCACTACACCGGGATGCTTGAAGAT | KVDSSRDRNKPFKFMLGK |
| | GGAAAGAAAGTTGATTCCTCCCGGGACAGAAACAAG | QEVIRGWEEGVAQMSVGQ |
| | CCCTTTAAGTTTATGCTAGGCAAGCAGGAGGTGATC | RAKLTISPDYAYGATGHPGI |
| | CGAGGCTGGGAAGAAGGGGTTGCCCAGATGAGTGT | IPPHATLVFDVELLKLE |
| | GGGTCAGAGAGCCAAACTGACTATATCTCCAGATTA | |
| | TGCCTATGGTGCCACTGGGCACCCAGGCATCATCC | |
| | CACCACATGCCACTCTCGTCTTCGATGTGGAGCTTC | |
| | TAAAACTGGAA | |
| Linker | SEQ ID NO: 3 | SEQ ID NO: 4 |
| | TCTGGCGGTGGATCCGGA | SGGGSG |
| ΔCaspase9 | SEQ ID NO: 5 | SEQ ID NO: 6 |
| | GTCGACGGATTTGGTGATGTCGGTGCTCTTGAGAGT | VDGFGDVGALESLRGNAD |
| | TTGAGGGGAAATGCAGATTTGGCTTACATCCTGAGC | LAYILSMEPCGHCLIINNVN |
| | ATGGAGCCCTGTGGCCACTGCCTCATTATCAACAAT | FCRESGLRTRTGSNIDCEK |
| | GTGAACTTCTGCCGTGAGTCCGGGCTCCGCACCCG | LRRRFSSLHFMVEVKGDLT |
| | CACTGGCTCCAACATCGACTGTGAGAAGTTGCGGC | AKKMVLALLELARQDHGAL |
| | GTCGCTTCTCCTCGCTGCATTTCATGGTGGAGGTGA | DCCVVVILSHGCQASHLQF |
| | AGGGCGACCTGACTGCCAAGAAAATGGTGCTGGCT | PGAVYGTDGCPVSVEKIVN |
| | TTGCTGGAGCTGGCGCGGCAGGACCACGGTGCTCT | IFNGTSCPSLGGKPKLFFIQ |
| | GGACTGCTGCGTGGTGGTCATTCTCTCTCACGGCTG | ACGGEQKDHGFEVASTSP |
| | TCAGGCCAGCCACCTGCAGTTCCCAGGGGCTGTCT | EDESPGSNPEPDATPFQE |
| | ACGGCACAGATGGATGCCCTGTGTCGGTCGAGAAG | GLRTFDQLDAISSLPTPSDI |
| | ATTGTGAACATCTTCAATGGGACCAGCTGCCCCAGC | FVSYSTFPGFVSWRDPKS |
| | CTGGGAGGGAAGCCCAAGCTCTTTTTCATCCAGGCC | GSWYVETLDDIFEQWAHS |
| | TGTGGTGGGGAGCAGAAAGACCATGGGTTTGAGGT | EDLQSLLLRVANAVSVKGI |
| | GGCCTCCACTTCCCCTGAAGACGAGTCCCCTGGCA | YKQMPGCFNFLRKKLFFKT |
| | GTAACCCCGAGCCAGATGCCACCCCGTTCCAGGAA | SASRA |
| | GGTTTGAGGACCTTCGACCAGCTGGACGCCATATCT | |
| | AGTTTGCCCACACCCAGTGACATCTTTGTGTCCTACT | |
| | CTACTTTCCCAGGTTTTGTTTCCTGGAGGGACCCCA | |
| | AGAGTGGCTCCTGGTACGTTGAGACCCTGGACGAC | |
| | ATCTTTGAGCAGTGGGCTCACTCTGAAGACCTGCAG | |
| | TCCCTCCTGCTTAGGGTCGCTAATGCTGTTTCGGTG | |
| | AAAGGGATTTATAAACAGATGCCTGGTTGCTTTAATT | |
| | TCCTCCGGAAAAAACTTTTCTTTAAAACATCAGCTAG | |
| | CAGAGCC | |
| T2A | SEQ ID NO: 7 | SEQ ID NO: 8 |
| | GAGGGCAGGGGAAGTCTTCTAACATGCGGGGACGT | EGRGSLLTCGDVEENPGP |
| | GGAGGAAAATCCCGGGCCC | |
| ΔCD19 | SEQ ID NO: 9 | SEQ ID NO: 10 |
| | ATGCCACCTCCTCGCCTCCTCTTCTTCCTCCTCTTCC | MPPPRLLFFLLFLTPMEVR |
| | TCACCCCCATGGAAGTCAGGCCCGAGGAACCTCTA | PEEPLVVKVEEGDNAVLQC |
| | GTGGTGAAGGTGGAAGAGGGAGATAACGCTGTGCT | LKGTSDGPTQQLTWSRES |
| | GCAGTGCCTCAAGGGGACCTCAGATGGCCCCACTC | PLKPFLKLSLGLPGLGIHMR |
| | AGCAGCTGACCTGGTCTCGGGAGTCCCCGCTTAAA | PLAIWLFIFNVSQQMGGFY |
| | CCCTTCTTAAAACTCAGCCTGGGGCTGCCAGGCCTG | LCQPGPPSEKAWQPGWT |
| | GGAATCCACATGAGGCCCCTGGCCATCTGGCTTTTC | VNVEGSGELFRWNVSDLG |
| | ATCTTCAACGTCTCTCAACAGATGGGGGGCTTCTAC | GLGCGLKNRSSEGPSSPS |
| | CTGTGCCAGCCGGGGCCCCCCTCTGAGAAGGCCTG | GKLMSPKLYVWAKDRPEI |
| | GCAGCCTGGCTGGACAGTCAATGTGGAGGGCAGCG | WEGEPPCLPPRDSLNQSL |
| | GGGAGCTGTTCCGGTGGAATGTTTCGGACCTAGGT | SQDLTMAPGSTLWLSCGV |
| | GGCCTGGGCTGTGGCCTGAAGAACAGGTCCTCAGA | PPDSVSRGPLSWTHVHPK |
| | GGGCCCCAGCTCCCCTTCCGGGAAGCTCATGAGCC | GPKSLLSLELKDDRPARDM |
| | CCAAGCTGTATGTGTGGGCCAAAGACCGCCCTGAG | WVMETGLLLPRATAQDAG |
| | ATCTGGGAGGGAGAGCCTCCGTGTCTCCCACCGAG | KYYCHRGNLTMSFHLEITA |
| | GGACAGCCTGAACCAGAGCCTCAGCCAGGACCTCA | RPVLWHWLLRTGGWKVSA |
| | CCATGGCCCCTGGCTCCACACTCTGGCTGTCCTGTG | VTLAYLIFCLCSLVGILHLQ |
| | GGGTACCCCCTGACTCTGTGTCCAGGGGCCCCCTC | RALVLRRKRKRMTDPTRR |
| | TCCTGGACCCATGTGCACCCCAAGGGGCCTAAGTC | F* |
| | ATTGCTGAGCCTAGAGCTGAAGGACGATCGCCCGG | |
| | CCAGAGATATGTGGGTAATGGAGACGGGTCTGTTGT | |
| | TGCCCCGGGCCACAGCTCAAGACGCTGGAAAGTAT | |
| | TATTGTCACCGTGGCAACCTGACCATGTCATTCCAC | |
| | CTGGAGATCACTGCTCGGCCAGTACTATGGCACTGG | |
| | CTGCTGAGGACTGGTGGCTGGAAGGTCTCAGCTGT | |
| | GACTTTGGCTTATCTGATCTTCTGCCTGTGTTCCCTT | |
| | GTGGGCATTCTTCATCTTCAAAGAGCCCTGGTCCTG | |
| | AGGAGGAAAAGAAAGCGAATGACTGACCCCACCAG | |
| | GAGATTCTAA | |

### Example 6: Representative Embodiments

A1. A compound of the following Formula I

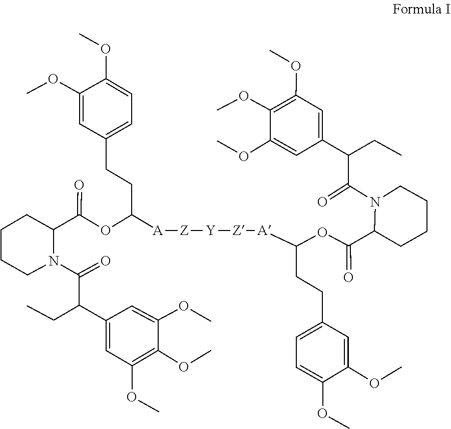

or a pharmaceutically acceptable salt thereof, wherein:
Z and Z′ are the same or different and each independently is O, NR¹², —N—, S, SO, SO₂ or CH₂;
Y is L, M or Q:

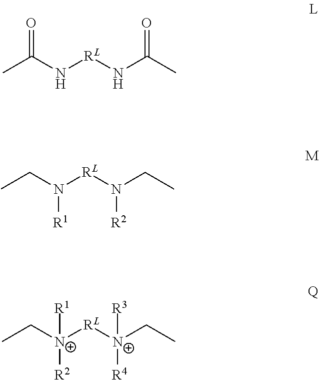

R¹, R², R³, and R⁴ are the same or different, and each is independently hydrogen, lower alkyl, heteroalkyl, perhaloalkyl, lower alkoxy, lower cycloalkyl, lower aryl, cycloalkyl, aryl, lower heterocycloalkyl, heterocycloalkyl, lower heteroaryl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when Y is M, R¹ and R² together with —N—R_{L}—N— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, cycloalkyl, heterocycloalkyl, heteroaryl, heteroarylalkyl, alkylsulfonyl, sulfonamide, alkylsulfonamido and alkylsilyloxy;
when Y is Q, R¹ and R² together with N⁺ may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, cycloalkyl, heterocycloalkyl, heteroaryl, heteroarylalkyl, alkylsulfonyl, sulfonamide, alkylsulfonamido and alkylsilyloxy;
when Y is Q, R³ and R⁴ together with N⁺ may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when Y is Q, R¹ and R³ together with —N⁺—R_{L}—N⁺— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, cycloalkyl, heterocycloalkyl, heteroaryl, heteroarylalkyl, alkylsulfonyl, sulfonamide, alkylsulfonamido and alkylsilyloxy;
when Y is Q, R² and R⁴ together with —N⁺—R_{L}—N⁺— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
   when Y is Q, one of R¹, R², R³ and R⁴ may be nonexistent;
R_{L} is a lower alkylene, alkenylene, alkynylene, acyl, cycloalkyl, or aryl, in which none or one or more carbon atoms are replaced by O, NR¹³, S, SO, SO₂, and which is optionally substituted with hydroxyl, alkoxyl, amino, alkylamino, thiol, thioalkyl, or halogen;
A and A′ are the same or different and each independently are

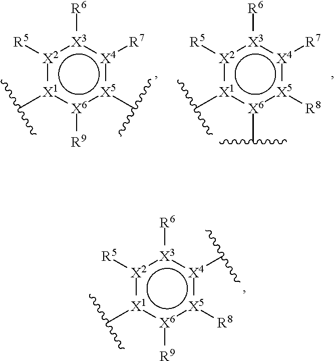

thiophene, furan, pyrrole, carbonyl, lower dialkyl ether, lower dialkyl thioether, lower dialkylamino, cyclopropylene, alkanylene, cycloalkanylene, alkenylene, cycloalkenylene, lower alkynylene, lower cycloalkynylene, carbamate, sulfanyl, sulfinyl, sulfonyl, thiocarbonyl, imino, or hydroxyimino, in which independently none or one or more carbon atoms are replaced by O, NR¹⁴, S, SO, SO₂, and which is optionally substituted with hydroxyl, alkoxyl, amino, alkylamino, thiol, thioalkyl, or halogen;
X¹, X², X³, X⁴, X⁵ and X⁶ independently are carbon or nitrogen with the proviso that none, one, two or three of X¹, X², X³, X⁴, X⁵ and X⁶ are nitrogen;
when X², X³, X⁴, X⁵ or X⁶ is carbon, R⁵, R⁶, R⁷, R⁸ or R⁹, respectively, independently is hydrogen, hydroxyl, halogen, C1-C2 alkyl or C1-C2 alkyl substituted with hydroxyl, halogen or NR¹⁰R¹¹;
R¹⁰ and R¹¹ independently are hydrogen or C1-C2 alkyl;
R12 is hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
R¹³ is hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
R¹⁴ is hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when X², X³, X⁴ or X⁶ is nitrogen, R⁵, R⁶, R⁷, R⁸ or R⁹, respectively, is not present or is hydrogen, C1-C2 alkyl or C1-C2 alkyl substituted with hydroxyl, halogen or NR¹⁰R¹¹;
with the proviso that (i) Y is M or Q when A and A′ are phenyl and Z and Z′ are oxygen, or (ii) A and A′ are not the same, or one or both of A and A′ are not phenyl, or Z and Z′ are not the same, or one or both of Z and Z′ are not oxygen, when Y is L and R_{L} is —CH₂—CH₂—.
   A1.1. The compound of embodiment A1, wherein
R¹, R², R³, and R⁴ are the same or different, and each is independently hydrogen, lower alkyl, heteroalkyl, perhaloalkyl, lower alkoxy, lower cycloalkyl, lower aryl, cycloalkyl, aryl, lower heterocycloalkyl, heterocycloalkyl, lower heteroaryl, or heteroaryl, which independently are optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower cycloalkyl, lower heterocycloalkyl, lower heteroaryl, lower heteroarylalkyl, lower alkylsulfonyl, sulfonamide, lower alkylsulfonamido and lower alkylsilyloxy;
when Y is M, R¹ and R² together with —N—R_{L}—N— may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heteroaryl, lower heterocycloalkyl, lower heteroaryl, lower heteroarylalkyl, lower alkylsulfonyl, sulfonamide, lower alkylsulfonamido and lower alkylsilyloxy;
when Y is Q, R¹ and R² together with N⁺ may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl lower heteroaryl, lower heteroarylalkyl, lower alkylsulfonyl, sulfonamide, lower alkylsulfonamido and lower alkylsilyloxy;
when Y is Q, R³ and R⁴ together with N⁺ may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl, lower heteroaryl, lower heteroarylalkyl, lower alkylsulfonyl, sulfonamide, lower alkylsulfonamido and lower alkylsilyloxy;
when Y is Q, R¹ and R³ together with —N⁺—R_{L}—N⁺— may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl, lower heteroaryl, lower heteroarylalkyl, lower alkylsulfonyl, sulfonamide, lower alkylsulfonamido and lower alkylsilyloxy;
when Y is Q, R² and R⁴ together with —N⁺—R_{L}—N⁺— may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkyl, lower heterocycloalkyl lower heteroaryl, lower heteroarylalkyl, lower alkylsulfonyl, sulfonamide, lower alkylsulfonamido and lower alkylsilyloxy;
when Y is Q, one of R¹, R², R³ and R⁴ may be nonexistent;
R_{L} is a lower alkylene, alkenylene, alkynylene, acyl, cycloalkyl, or aryl, in which none or one or more carbon atoms are replaced by O, NR¹³, S, SO, SO₂, and which is optionally substituted with hydroxyl, alkoxyl, amino, alkylamino, thiol, thioalkyl, or halogen;
A and A′ are the same or different and each independently are

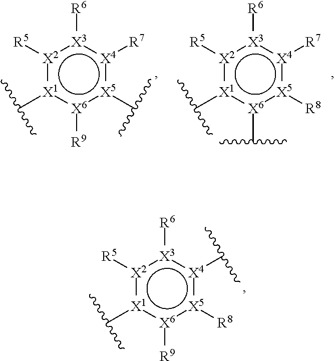

thiophene, furan, pyrrole, carbonyl, lower dialkyl ether, lower dialkyl thioether, lower dialkylamino, cyclopropylene, alkanylene, cycloalkanylene, alkenylene, cycloalkenylene, lower alkynylene, lower cycloalkynylene, carbamate, sulfanyl, sulfinyl, sulfonyl, thiocarbonyl, imino, or hydroxyimino, in which independently none or one or more carbon atoms are replaced by O, NR¹⁴, S, SO, SO₂, and which is optionally substituted with hydroxyl, lower alkoxyl, amino, lower alkylamino, thiol, lower thioalkyl, or halogen;
X¹, X², X³, X⁴, X⁵ and X⁶ independently are carbon or nitrogen with the proviso that none, one, two or three of X¹, X², X³, X⁴, X⁵ and X⁶ are nitrogen;
when X², X³, X⁴, X⁵ or X⁶ is carbon, R⁵, R⁶, R⁷, R⁸ or R⁹, respectively, independently is hydrogen, hydroxyl, halogen, C1-C2 alkyl or C1-C2 alkyl substituted with hydroxyl, halogen or NR¹⁰R¹¹;
R¹⁰ and R¹¹ independently are hydrogen or C1-C2 alkyl;
when X², X³, X⁴ or X⁶ is nitrogen, R⁵, R⁶, R⁷, R⁸ or R⁹, respectively, is not present or is hydrogen, C1-C2 alkyl or C1-C2 alkyl substituted with hydroxyl, halogen or NR¹⁰R¹¹;
with the proviso that (i) Y is M or Q when A and A′ are phenyl and Z and Z′ are oxygen, or (ii) A and A′ are not the same, or one or both of A and A′ are not phenyl, or Z and Z′ are not the same, or one or both of Z and Z′ are not oxygen, when Y is L and R_{L} is —CH₂—CH₂—.
   A1.2. The compound of embodiment A1, wherein
R¹, R², R³, and R⁴ are the same or different, and each is independently hydrogen, lower alkyl, lower heteroalkyl, lower perhaloalkyl, lower alkoxy, lower cycloalkyl, lower aryl, lower heterocycloalkyl, or lower heteroaryl, which independently are optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl, lower heteroaryl, lower heteroarylalkyl, lower alkylsulfonyl, sulfonamide, lower alkylsulfonamido and lower alkylsilyloxy;
when Y is M, R¹ and R² together with —N—R_{L}—N— may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl, lower heteroaryl, lower heteroarylalkyl, lower alkylsulfonyl, sulfonamide, lower alkylsulfonamido and lower alkylsilylox;
when Y is Q, R¹ and R² together with N⁺ may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl, lower heteroaryl, lower heteroarylalkyl, lower alkylsulfonyl, sulfonamide, lower alkylsulfonamido and lower alkylsilylox;
when Y is Q, R³ and R⁴ together with N⁺ may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl, lower heteroaryl, lower heteroarylalkyl, lower alkylsulfonyl, sulfonamide, lower alkylsulfonamido and lower alkylsilylox;
when Y is Q, R¹ and R³ together with —N⁺—R_{L}—N⁺— may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl, lower heteroaryl, lower heteroarylalkyl, lower alkylsulfonyl, sulfonamide, lower alkylsulfonamido and lower alkylsilylox;
when Y is Q, R² and R⁴ together with —N⁺—R^{L}—N⁺— may form a heterocyclic or heteroaryl ring optionally substituted with one or more substituents chosen from halogen, hydroxy, alkyl, lower heteroalkyl, lower haloalkyl, lower perhaloalkyl, lower perhaloalkoxy, lower alkoxy, lower haloalkoxy, lower alkoxyalkyl, lower acyl, oxo, lower acyloxy, lower carboxyl, amido, cyano, amino, lower alkylamino, lower alkylaminoalkyl, thiol, lower alkylthio, lower alkylthioalkyl, lower haloalkylthio, lower perhaloalkylthio, nitro, lower aryl, lower arylalkyl, lower cycloalkylalkyl, lower heterocycle-alkyl, lower cycloalkyl, lower heterocycloalkyl, lower heteroaryl, lower heteroarylalkyl, lower alkylsulfonyl, sulfonamide, lower alkylsulfonamido and lower alkylsilylox;
when Y is Q, one of R¹, R², R³ and R⁴ may be nonexistent;
R_{L} is a lower alkylene, lower alkenylene, lower alkynylene, lower acyl or lower aryl, in which none or one or more carbon atoms are replaced by O, NR¹³, S, SO, SO₂, and which is optionally substituted with hydroxyl, lower alkoxyl, amino, lower alkylamino, thiol, lower thioalkyl, or halogen; A and A′ are the same or different and each independently are

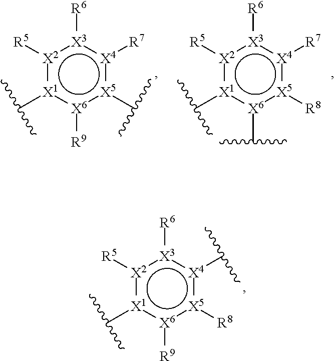

thiophene, furan, pyrrole, carbonyl, lower dialkyl ether, lower dialkyl thioether, lower dialkylamino, cyclopropylene, alkanylene, cycloalkanylene, alkenylene, cycloalkenylene, lower alkynylene, lower cycloalkynylene, carbamate, sulfanyl, sulfinyl, sulfonyl, thiocarbonyl, imino, or hydroxyimino, in which independently none or one or more carbon atoms are replaced by O, NR¹⁴, S, SO, SO₂, and which is optionally substituted with hydroxyl, lower alkoxyl, amino, lower alkylamino, thiol, lower thioalkyl, or halogen;
X¹, X², X³, X⁴, X⁵ and X⁶ independently are carbon or nitrogen with the proviso that none, one, two or three of X¹, X², X³, X⁴, X⁵ and X⁶ are nitrogen;
when X², X³, X⁴, X⁵ or X⁶ is carbon, R⁵, R⁶, R⁷, R⁸ or R⁹, respectively, independently is hydrogen, hydroxyl, halogen, C1-C2 alkyl or C1-C2 alkyl substituted with hydroxyl, halogen or NR¹⁰R¹¹;
R¹⁰ and R¹¹ independently are hydrogen or C1-C2 alkyl;
when X², X³, X⁴ or X⁶ is nitrogen, R⁵, R⁶, R⁷, R⁸ or R⁹, respectively, is not present or is hydrogen, C1-C2 alkyl or C1-C2 alkyl substituted with hydroxyl, halogen or NR¹⁰R¹¹;
with the proviso that (i) Y is M or Q when A and A′ are phenyl and Z and Z′ are oxygen, or (ii) A and A′ are not the same, or one or both of A and A′ are not phenyl, or Z and Z′ are not the same, or one or both of Z and Z′ are not oxygen, when Y is L and R_{L} is —CH₂—CH₂—.
   A2. The compound of embodiment A1, of the following Formula II

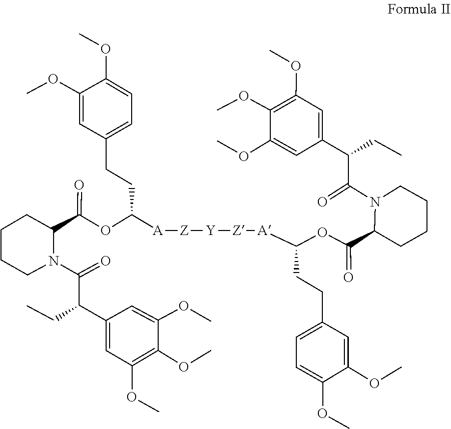

or a pharmaceutically acceptable salt thereof.
A3. The compound of any one of embodiments A1-A2, wherein R_{L} is —CH₂—CH₂—.
A3.1. The compound of any one of embodiments A1-A3, wherein Y is M.
A3.2. The compound of embodiment A3, wherein Y is

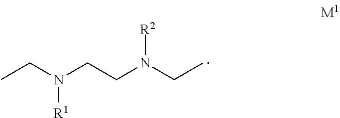

A4. The compound of any one of embodiments A3.1-A3.2, wherein R¹ and R² are the same.
A4.1. The compound of embodiment A3.1, wherein R¹ and R² are H.
A5. The compound of any one of embodiments A1-A2, wherein Y is Q.
A5.1. The compound of embodiment A5, wherein Q is

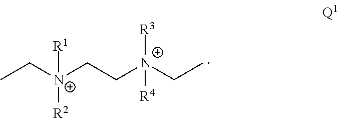

A6. The compound of embodiment A5 or A5.1, wherein R¹ and R³ are the same, and R² and R⁴ are the same.
A7. The compound of any one of embodiments A1-A6, wherein A and A′ are the same.
A7.1. The compound of any one of embodiments A5-A5.1, wherein R¹, R², R³, and R⁴ are H.
A7.2. The compound of any one of embodiments A5-A5.1, wherein one of R¹, R², R³ and R⁴ is nonexistent.
A7.3. The compound of any one of embodiments A5-A5.1, wherein R¹ is nonexistent and R², R³ and R⁴ are H, or R³ is nonexistent and R¹, R² and R⁴ are H.
A8. The compound of any one of embodiments A1-A7.3, wherein A and A′ independently are phenyl, pyridinyl, methylpyridinyl, pyridazinyl, pyrimidinyl, pyrazinyl, triazinyl, substituted phenyl, substituted pyridinyl, substituted pyridazinyl, substituted pyrimidinyl, substituted pyrazinyl or substituted triazinyl.
A9. The compound of any one of embodiments A1-A7.3, wherein:
one or both of A and A′ is

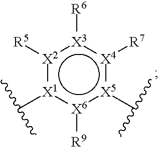

X², X⁴ and X⁶ are carbon; and
one of X¹, X³ and X⁵ is nitrogen and two of X¹, X³ and X⁵ are carbon, or
two of X¹, X³ and X⁵ are nitrogen and one of X¹, X³ and X⁵ is carbon, or
X¹, X³ and X⁵ are nitrogen.
   A10. The compound of any one of embodiments A1-A7.3, wherein:
one or both of A and A′ is

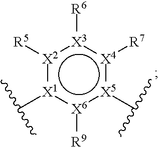

five of X¹, X², X³, X⁴, X⁵ and X⁶ are carbon; and
one of X¹, X², X³, X⁴, X⁵ and X⁶ is nitrogen.
   A11. The compound of any one of embodiments A1-A7, wherein:
one or both of A and A′ is

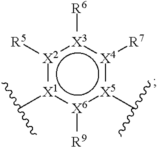

X¹, X², X⁴, X⁵ and X⁶ are carbon;
R⁵, R⁷ and R⁹ are hydrogen; and
R⁶ is methyl.
   A12. The compound of any one of embodiments A1-A7.3, wherein:
one or both of A and A′ is

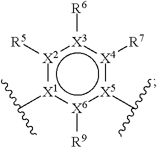

X¹, X², X⁴, X⁵ and X⁶ are carbon;
X³ is nitrogen;
R⁵, R⁷ and R⁹ are hydrogen; and
R⁶ is methyl.
   A12.1. The compound of any one of embodiments A1-A7.3, wherein:
one or both of A and A′ is

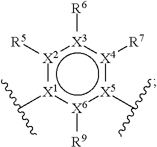

X¹, X², X⁴, X⁵ and X⁶ are carbon;
X³ is nitrogen; and
R⁵, R⁶, R⁷ and R⁹ are hydrogen.
   A13. The compound of any one of embodiments A1-A7.3, wherein:
one or both of A and A′ is

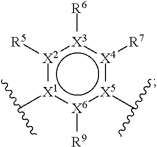

and
X¹ and X⁵ are carbon.
   A14. The compound of any one of embodiments A1-A7.3, wherein:
one or both of A and A′ is

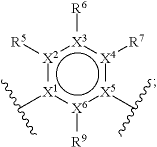

and
X¹, X², X³, X⁴, X⁵ and X⁶ are carbon.
   A15. The compound of embodiment A14, wherein, R⁵, R⁷ and R⁹ are hydrogen, and R⁶ is methyl.
   A16. The compound of any one of embodiments A1-A7.3, wherein:
one or both of A and A′ is

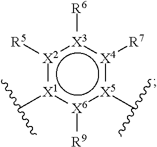

and
X¹ or X⁵, or X¹ and X⁵, are nitrogen.
   A17. The compound of any one of embodiments A1-A7.3, wherein:
one or both of A and A′ is

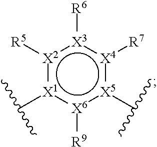

and
one, two or three of X¹, X³ and X⁵, are nitrogen.
   A18. The compound of any one of embodiments A1-A17, wherein one or more of R⁵, R⁶, R⁷, R⁸, and R⁹ are halogen.
   A19. The compound of embodiment A18, wherein the halogen is F or Cl.
   A20. The compound of any one of embodiments A1-A19, wherein Z and Z′ are O.
   A21. The compound of any one of embodiments A3.1-A4.1 or A7-A20, wherein each of R¹ and R² independently is hydrogen, C1-C2 alkyl, or C1-C2 alkyl substituted with one or more halogen atoms.
   A21.1. The compound of embodiment A21, wherein both R¹ and R² are CH₃ or C₂H₅.
   A21.2. The compound of embodiment A21, wherein both R¹ and R² are CH₃.
   A22. The compound of embodiment A20, wherein each of R¹ and R³ independently is hydrogen, CH₃ or C₂H₅.
   A23. The compound of any one of embodiments A1-A2 or A5-A20, wherein each of R¹, R², R³, and R⁴ independently is hydrogen, C1-C2 alkyl, or C1-C2 alkyl substituted with one or more halogen atoms.
   A24. The compound of embodiment A23, wherein each of R¹, R², R³, and R⁴ independently is hydrogen, CH₃ or C₂H₅.
   A25. The compound of any one of embodiments A1-A24, wherein A and A′ are phenyl and Y is M or M¹.
   A26. The compound of embodiment A25, wherein each of R¹ and R² independently is hydrogen, CH₃ or C₂H₅.
   A27. The compound of any one of embodiments A1-A4.1, or A7-A26, wherein:
Z and Z′ are O;
Y is M;
R¹ and R² are CH₃; and
A and A′ are

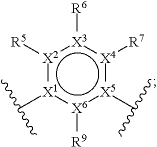

and are the same.
A27.1. The compound of embodiment A27, wherein:
X¹, X², X⁴, X⁵ and X⁶ are carbon;
X³ is nitrogen;
R⁵, R⁷ and R⁹ are hydrogen; and
R⁶ is CH₃.
   A28. The compound of any one of embodiments A1-A2, wherein A and A′ are phenyl, Y is M, and R¹ and R² are CH₃.
   A29. The compound of any one of embodiments A1-A2, wherein A and A′ are phenyl, Y is M, Z and Z′ are O, and R¹ and R² are C2H5.
   A30. The compound of any one of embodiments A1-A2, wherein A and A′ are pyridinyl, Y is M, Z and Z′ are O, and R¹ and R² are CH₃.
   A31. The compound of embodiment A2, having the following structural formula

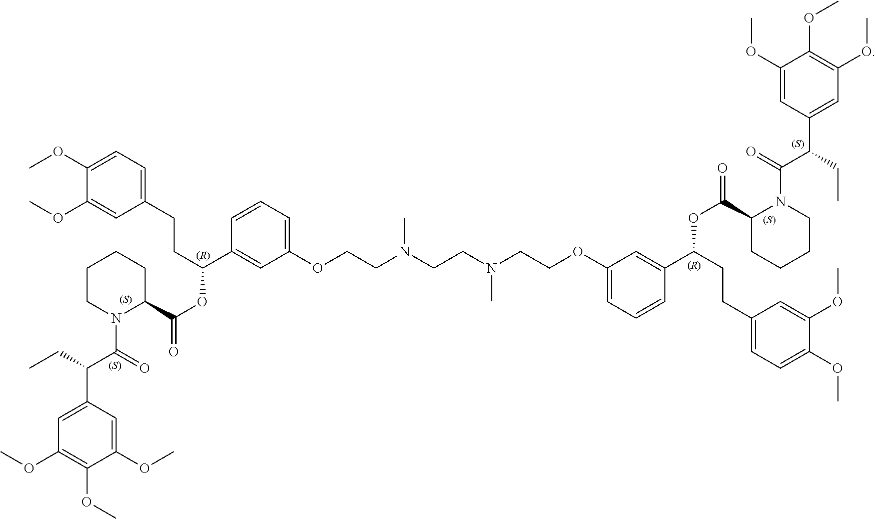

A32. The compound of any one of embodiments A1-A2, wherein Y is L.
A32.1. The compound of embodiment A32, wherein Y is

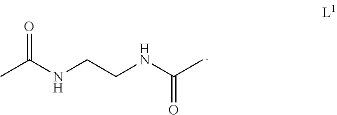

A33. The compound of embodiment A32 or A32.1, wherein A and A′ are

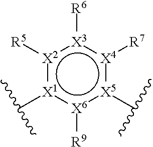

and are the same.
A34. The compound of embodiment A33, wherein one, two or three of X¹, X², X³, X⁴, X⁵ and X⁶ are nitrogen.
A35. The compound of embodiment A33 or A35, wherein five of X¹, X², X³, X⁴, X⁵ and X⁶ are carbon and one of X¹, X², X³, X⁴, X⁵ and X⁶ is nitrogen.
A35.1. The compound of embodiment A33 or A35, wherein X¹, X², X⁴, X⁵ and X⁶ are carbon and X³ is nitrogen.
A35.2. The compound of embodiment A35.1, wherein R⁶ is CH₃ or C₂H₅.
A35.3. The compound of embodiment A35.1, wherein R⁶ is CH₃.
A36. The compound of any one of embodiments A33-A35.3, wherein X¹ and X⁵ are carbon.
A37. The compound of any one of embodiments A33-A36, wherein A and A′ are substituted with a halogen.
A38. The compound of embodiment A37, wherein the halogen is F or Cl.
A39. The compound of any one of embodiments A33-A38, wherein Z and Z′ are O.
A40. The compound of any one of embodiments A1-A2, wherein R⁵, R⁶, R⁷ or R⁸ is not present or is hydrogen or methyl.
B1. A compound of any one of embodiments A1-A40, which selectively binds to a FKBP12 polypeptide mutant comprising an amino acid substitution at a position corresponding to position 36 in the wild type FKBP12 polypeptide.
B2. The compound of embodiment B1, wherein the amino acid substitution is to an amino acid chosen from valine, leucine, isoleucine and alanine.
B3. The compound of embodiment B1 or B2, wherein the amino acid substitution is to valine.
B4. The compound of any one of embodiments B1-B3, wherein the FKBP12 polypeptide mutant is FKBP12v36.
B5. The compound of any one of embodiments B1-B4, which binds to the FKBP12 polypeptide mutant with an IC₅₀ at least 10 times lower than the IC₅₀ of the compound binding to the wild type FKBP12 polypeptide.
B6. The compound of any one of embodiments B1-B4, which binds to the FKBP12 polypeptide mutant with an IC₅₀ at least 100 times lower than the IC₅₀ of the compound binding to the wild type FKBP12 polypeptide.
B7. The compound of any one of embodiments B1-B4, which binds to the FKBP12 polypeptide mutant with an IC₅₀ at least 1000 times lower than the IC₅₀ of the compound binding to the wild type FKBP12 polypeptide.
B8. The compound of any one of embodiments B1-B7, which has a binding affinity (IC₅₀) to FKBP12v36 of 100 nM or less.
C1. A compound of any one of embodiments A1-A40 and B1-B8, which is a pharmaceutically acceptable salt comprising a counter ion chosen from phosphate, hydrochloride, besylate, benzoate, carbonate, chloride, citrate, diphosphate, estolate, fumarate, gluconate, malate, maleate, pamoate, stearate, succinate, sulfate, sulfonate, tartrate, tosylate, and valerate.
C2. The compound of embodiment C1, wherein the counter ion is phosphate.
C3. The compound of embodiment C1, wherein the counter ion is hydrochloride.
C3.1. A compound of embodiment A31, which is a pharmaceutically acceptable salt comprising a counter ion chosen from phosphate, hydrochloride, besylate, benzoate, carbonate, chloride, citrate, diphosphate, estolate, fumarate, gluconate, malate, maleate, pamoate, stearate, succinate, sulfate, sulfonate, tartrate, tosylate, and valerate.
C3.2. The compound of embodiment C3.1, wherein the counter ion is phosphate.
C3.3. The compound of embodiment C3.1, wherein the counter ion is hydrochloride
C4. The compound of any one of embodiments A1-A40, B1-B8 and C1-C3.3, wherein the compound is soluble in water.
C5. The compound of embodiment C4, which is soluble in acetate buffer having a pH of 6 or less.
C6. The compound of embodiment C4 or C5, which is soluble in acetate buffer having a pH of 4 or less.
C6.1. The compound of embodiment C4, which is soluble in phosphate buffer having a pH of 6 or less.
C6.2. The compound of embodiment C4 or C5, which is soluble in phosphate buffer having a pH of 4 or less.
C7. The compound of any one of embodiments A1-A40, B1-B8 and C1-C6.2, which has a greater solubility in water than rimiducid.
C8. The compound of any one of embodiments A1-A40, B1-B8 and C1-C7, which has a solubility greater than 1 mg·mL⁻¹ in water.
C9. The compound of any one of embodiments A1-A40, B1-B8 and C1-C7, which has a solubility greater than 2.5 mg·mL⁻¹ in water.
C10. The compound of any one of embodiments A1-A40, B1-B8 and C1-C7, which has a solubility greater than 0.2 mg·mL⁻¹ in acetate buffer having a pH of 6 or less.
C11. The compound of any one of embodiments A1-A40, B1-B8 and C1-C7, which has a solubility greater than 4 mg·mL⁻¹ in acetate buffer having a pH of 6 or less.
C12. The compound of any one of embodiments A1-A40, B1-B8 and C1-C7, which has a solubility greater than 0.2 mg·mL⁻¹ in acetate buffer having a pH of 4 or less.
C13. The compound of any one of embodiments A1-A40, B1-B8 and C1-C7, which has a solubility greater than 4 mg·mL⁻¹ in acetate buffer having a pH of 4 or less.
C13.1. The compound of any one of embodiments A1-A40, B1-B8 and C1-C7, which has a solubility greater than 0.2 mg·mL⁻¹ in phosphate buffer having a pH of 6 or less.
C13.2. The compound of any one of embodiments A1-A40, B1-B8 and C1-C7, which has a solubility greater than 4 mg·mL⁻¹ in phosphate buffer having a pH of 6 or less.
C13.3. The compound of any one of embodiments A1-A40, B1-B8 and C1-C7, which has a solubility greater than 0.2 mg·mL⁻¹ in phosphate buffer having a pH of 4 or less.
C13.4. The compound of any one of embodiments A1-A40, B1-B8 and C1-C7, which has a solubility greater than 4 mg·mL⁻¹ in phosphate buffer having a pH of 4 or less.
C14. A pharmaceutical composition comprising a compound of any one of embodiments A1-A40, B1-B8 and C1-C13.4, and a pharmaceutically acceptable excipient or carrier.
C15. The pharmaceutical composition of embodiment C14, which is a liquid composition.
C16. The pharmaceutical composition of embodiment C15, which is 80% or greater (weight/weight) water.
C17. The pharmaceutical composition of embodiment C14, which is a dry powder composition.
C18. The pharmaceutical composition of embodiment C17, comprising a pharmaceutically acceptable co-polymer.
C19. The pharmaceutical composition of embodiment C18, wherein the co-polymer is chosen from poly(vinyl alcohol), poly(vinyl pyrrolidone), hypromellose, acetate, and succinate, and combinations thereof.
C20. The pharmaceutical composition of any one of embodiments C17-C19, which is a spray dried composition.
C21. The pharmaceutical composition of any one of embodiments C14-C20, wherein the excipient or carrier is chosen from polyethylene glycol (PEG), polysorbate, ethanol, glycerol, glycerin, sorbitol, glucose, sucrose, dimethylacetamide, and dimethylsulfoxide (DMSO) and combinations thereof.
D1. A method of multimerizing polypeptides expressed in a cell, comprising contacting the cell with a compound of any one of embodiments A1-A40, B1-B8 and C1-C13.4, or with a pharmaceutical composition of any one of embodiments C14-C20, wherein the polypeptides comprise at least one FKBP12 mutant polypeptide.
D2. The method of embodiment D1, wherein the at least one FKBP12 mutant polypeptide comprises an amino acid substitution at a position corresponding to position 36 in the wild type FKBP12 polypeptide.
D3. The method of embodiment D2, wherein the amino acid substitution is to an amino acid chosen from valine, leucine, isoleucine and alanine.
D4. The method of any one of embodiments D1-D3, wherein the amino acid substitution is to valine.
D5. The method of embodiment D4, wherein the FKBP12 mutant polypeptide is FKBP12v36.
D6. The method of any one of embodiments D1-D4, wherein the at least one FKBP12 mutant polypeptide is Fv′Fvls.

The entirety of each patent, patent application, publication and document referenced herein hereby is incorporated by reference for all purposes. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. Still further, the terms “having”, “including”, “containing” and “comprising” are interchangeable and one of skill in the art is cognizant that these terms are open ended terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A compound of the following Formula I 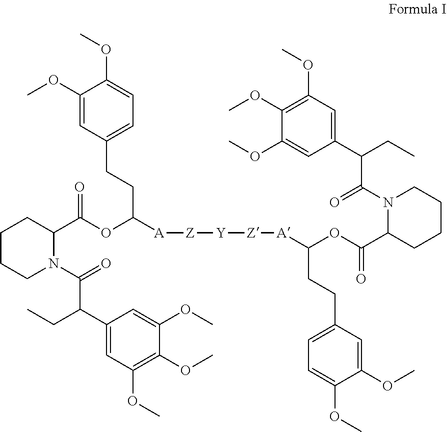
or a pharmaceutically acceptable salt thereof, wherein:
Z and Z′ are the same or different and each independently is O, NR¹², —N—, S, SO, SO₂ or CH₂;
Y is L, M or Q: 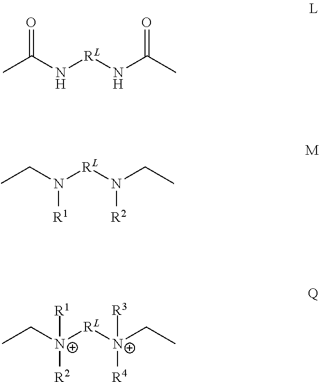
R¹, R², R³, and R⁴ are the same or different, and each is independently hydrogen, lower alkyl, heteroalkyl, perhaloalkyl, lower alkoxy, lower cycloalkyl, lower aryl, cycloalkyl, aryl, lower heterocycloalkyl, heterocycloalkyl, lower heteroaryl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when Y is M, R¹ and R² together with —N—R_{L}—N— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, cycloalkyl, heterocycloalkyl, heteroaryl, heteroarylalkyl, alkylsulfonyl, sulfonamide, alkylsulfonamido and alkylsilyloxy;
when Y is Q, R¹ and R² together with N⁺ may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, cycloalkyl, heterocycloalkyl, heteroaryl, heteroarylalkyl, alkylsulfonyl, sulfonamide, alkylsulfonamido and alkylsilyloxy;
when Y is Q, R³ and R⁴ together with N⁺ may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when Y is Q, R¹ and R³ together with —N⁺—R_{L}—N⁺— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, cycloalkyl, heterocycloalkyl, heteroaryl, heteroarylalkyl, alkylsulfonyl, sulfonamide, alkylsulfonamido and alkylsilyloxy;
when Y is Q, R² and R⁴ together with —N⁺—R_{L}—N⁺— may form a heterocyclic or heteroaryl ring optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when Y is Q, one of R¹, R², R³ and R⁴ may be nonexistent;
R_{L} is a lower alkylene, alkenylene, alkynylene, acyl, cycloalkyl, or aryl, in which none or one or more carbon atoms are replaced by O, NR¹³, S, SO, SO₂, and which is optionally substituted with hydroxyl, alkoxyl, amino, alkylamino, thiol, thioalkyl, or halogen;
A and A′ are the same or different and each independently are 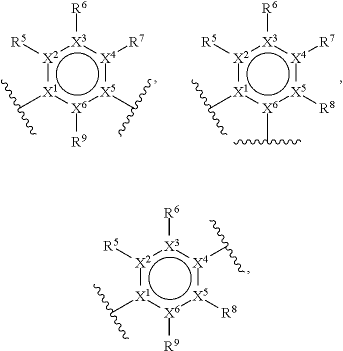
thiophene, furan, pyrrole, carbonyl, lower dialkyl ether, lower dialkyl thioether, lower dialkylamino, cyclopropylene, alkanylene, cycloalkanylene, alkenylene, cycloalkenylene, lower alkynylene, lower cycloalkynylene, carbamate, sulfanyl, sulfinyl, sulfonyl, thiocarbonyl, imino, or hydroxyimino, in which independently none or one or more carbon atoms are replaced by O, NR¹⁴, S, SO, SO₂, and which is optionally substituted with hydroxyl, alkoxyl, amino, alkylamino, thiol, thioalkyl, or halogen;
X¹, X², X³, X⁴, X⁵ and X⁶ independently are carbon or nitrogen with the proviso that none, one, two or three of X¹, X², X³, X⁴, X⁵ and X⁶ are nitrogen;
when X², X³, X⁴, X⁵ or X⁶ is carbon, R⁵, R⁶, R⁷, R⁸ or R⁹, respectively, independently is hydrogen, hydroxyl, halogen, C1-C2 alkyl or C1-C2 alkyl substituted with hydroxyl, halogen or NR¹⁰R¹¹;
R¹⁰ and R¹¹ independently are hydrogen or C1-C2 alkyl;
R¹² is hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
R¹³ is hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
R¹⁴ is hydrogen, lower alkyl, heteroalkyl, perhaloalkyl, lower alkoxy, lower cycloalkyl, lower aryl, cycloalkyl, aryl, lower heterocycloalkyl, lower heteroaryl, heterocycloalkyl, or heteroaryl, which independently are optionally substituted with one or more substituents chosen from halogen, hydroxy, alkyl, heteroalkyl, haloalkyl, perhaloalkyl, perhaloalkoxy, alkoxy, haloalkoxy, alkoxyalkyl, acyl, oxo, acyloxy, carboxyl, amido, cyano, amino, alkylamino, alkylaminoalkyl, thiol, alkylthio, alkylthioalkyl, haloalkylthio, perhaloalkylthio, nitro, aryl, arylalkyl, cycloalkylalkyl, heterocycle-alkyl, cycloalkyl, heterocycloalkyl, heteroaryl, heteroarylalkyl, alkylsulfonyl, sulfonamide, alkylsulfonamido and alkylsilyloxy;
when X², X³, X⁴ or X⁶ is nitrogen, R⁵, R⁶, R⁷, R⁸ or R⁹, respectively, is not present or is hydrogen, C1-C2 alkyl or C1-C2 alkyl substituted with hydroxyl, halogen or NR¹⁰R¹¹;
with the proviso that (i) Y is M or Q when A and A′ are phenyl and Z and Z′ are oxygen, or (ii) A and A′ are not the same, or one or both of A and A′ are not phenyl, or Z and Z′ are not the same, or one or both of Z and Z′ are not oxygen, when Y is L and R_{L} is —CH₂—CH₂—.

2. The compound of claim 1, of the following Formula II 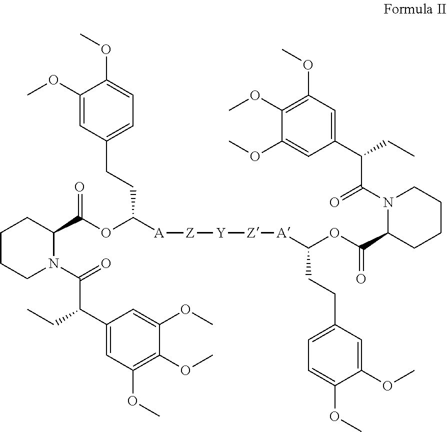
or a pharmaceutically acceptable salt thereof.

3. The compound of any one of claim 1 or 2, wherein R_{L} is —CH₂—CH₂—.

4. The compound of any one of claims 1 to 3, wherein Y is M or Y is

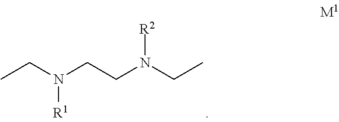

5. The compound of any one of claims 1 to 2, wherein Y is Q or Y is

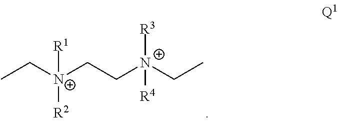

6. The compound of any one of claims 1 to 5, wherein:
one or both of A and A′ is 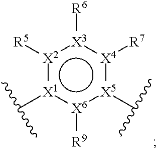
X², X⁴ and X⁶ are carbon; and
one of X¹, X³ and X⁵ is nitrogen and two of X¹, X³ and X⁵ are carbon, or
two of X¹, X³ and X⁵ are nitrogen and one of X¹, X³ and X⁵ is carbon, or
X¹, X³ and X⁵ are nitrogen.

7. The compound of any one of claims 1 to 5, wherein:
one or both of A and A′ is 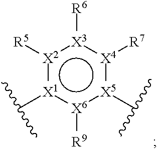
five of X¹, X², X³, X⁴, X⁵ and X⁶ are carbon; and
one of X¹, X², X³, X⁴, X⁵ and X⁶ is nitrogen.

8. The compound of any one of claims 1 to 5, wherein:
one or both of A and A′ is 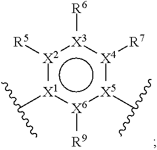
X¹, X², X⁴, X⁵ and X⁶ are carbon;
R⁵, R⁷ and R⁹ are hydrogen; and
R⁶ is methyl.

9. The compound of any one of claims 1 to 5, wherein:
one or both of A and A′ is 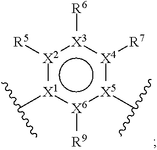
X¹, X², X⁴, X⁵ and X⁶ are carbon;
X³ is nitrogen;
R⁵, R⁷ and R⁹ are hydrogen; and
R⁶ is methyl.

10. The compound of any one of claims 1 to 5, wherein:
one or both of A and A′ is 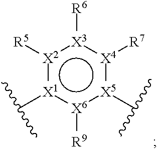
X¹, X², X⁴, X⁵ and X⁶ are carbon;
X³ is nitrogen; and
R⁵, R⁶, R⁷ and R⁹ are hydrogen.

11. The compound of any one of claims 1 to 5, wherein:
one or both of A and A′ is 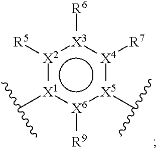
and
X¹ and X⁵ are carbon.

12. The compound of any one of claims 1 to 5, wherein:
one or both of A and A′ is 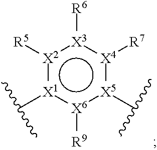
and
X¹, X², X³, X⁴, X⁵ and X⁶ are carbon.

13. The compound of claim 12, wherein, R⁵, R⁷ and R⁹ are hydrogen, and R⁶ is methyl.

14. The compound of any one of claims 1 to 5, wherein:
one or both of A and A′ is 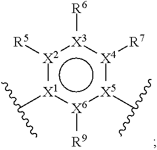
and
X¹ or X⁵, or X¹ and X⁵, are nitrogen.

15. The compound of any one of claims 1 to 5, wherein:
one or both of A and A′ is 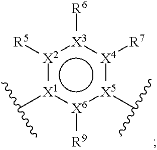
and
one, two or three of X¹, X³ and X⁵, are nitrogen.

16. The compound of any one of claims 1 to 15, wherein one or more of R⁵, R⁶, R⁷, R⁸, and R⁹ are halogen.

17. The compound of any one of embodiments 4 or 6 to 16, wherein each of R¹ and R² independently is hydrogen, C1-C2 alkyl, or C1-C2 alkyl substituted with one or more halogen atoms.

18. The compound of claim 17, wherein both R¹ and R² are CH₃ or C₂H₅.

19. The compound of any one of claims 1 to 18, wherein A and A′ are phenyl and Y is M or M¹.

20. The compound of claim 19, wherein each of R¹ and R² independently is hydrogen, CH₃ or C₂H₅.

21. The compound of any one of claims 1 to 4, or 16 to 20, wherein:
Z and Z′ are O;
Y is M;
R¹ and R² are CH₃; and
A and A′ are 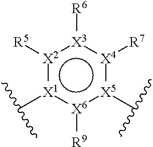
and are the same.

22. The compound of claim 21, wherein:
X¹, X², X⁴, X⁵ and X⁶ are carbon;
X³ is nitrogen;
R⁵, R⁷ and R⁹ are hydrogen; and
R⁶ is CH₃.

23. The compound of any one of claims 1 to 2, wherein A and A′ are phenyl, Y is M, and R¹ and R² are CH₃.

24. The compound of any one of claims 1 to 2, wherein A and A′ are phenyl, Y is M, Z and Z′ are O, and R¹ and R² are C₂H₅ or CH₃.

25. The compound of any one of claims 1 to 2, wherein Y is L or Y is

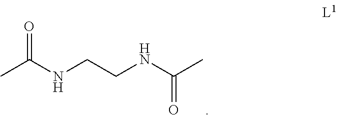

26. The compound of claim 25, wherein A and A′ are 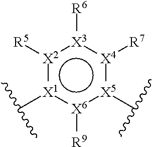
and are the same.

27. The compound of claim 26, wherein one, two or three of X¹, X², X³, X⁴, X⁵ and X⁶ are nitrogen; five of X¹, X², X³, X⁴, X⁵ and X⁶ are carbon and one of X¹, X², X³, X⁴, X⁵ and X⁶ is nitrogen; or X¹, X², X⁴, X⁵ and X⁶ are carbon and X³ is nitrogen.

28. The compound of claim 26, wherein X¹, X², X⁴, X⁵ and X⁶ are carbon and X³ is nitrogen, and R⁶ is CH₃ or C₂H₅.

29. The compound of any one of claims 26 to 28, wherein A and A′ are substituted with a halogen.

30. The compound of any one of claims 26 to 29, wherein Z and Z′ are O.

31. The compound of any one of claims 1 to 2, wherein R⁵, R⁶, R⁷ or R⁸ is not present or is hydrogen or methyl.

32. The compound of claim 2, having the following structural formula

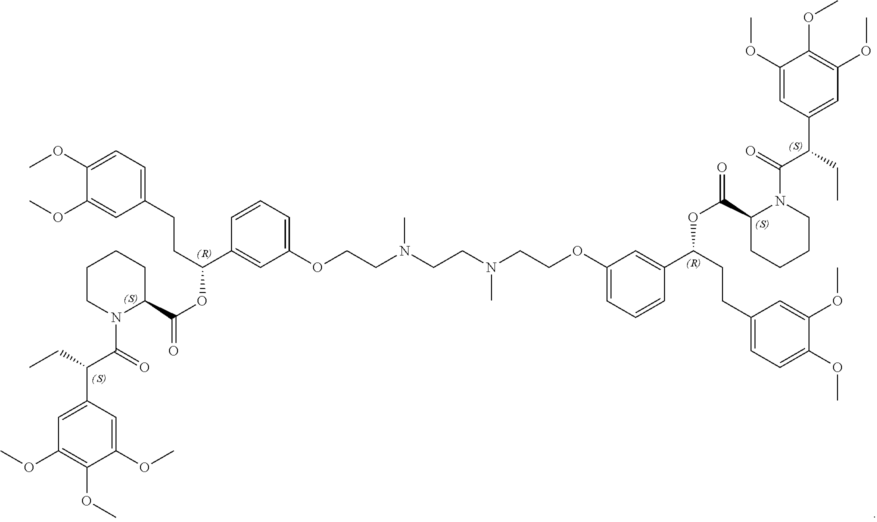

33. A compound of any one of claims 1 to 32, which selectively binds to a FKBP12 polypeptide mutant comprising FKBP12v36.

34. The compound of claim 33, which binds to FKBP12v36 with an IC₅₀ at least 10 times lower than the IC₅₀ of the compound binding to the wild type FKBP12 polypeptide.

35. The compound of any one of claims 33 to 34, which binds to the FKBP12 polypeptide mutant with an IC₅₀ at least 100 times lower than the IC₅₀ of the compound binding to the wild type FKBP12 polypeptide.

36. The compound of any one of claims 33 to 35, which has a binding affinity (IC₅₀) to FKBP12v36 of 100 nM or less.

37. A compound of any one of claims 1 to 36, which is a pharmaceutically acceptable salt comprising a counter ion chosen from phosphate, hydrochloride, besylate, benzoate, carbonate, chloride, citrate, diphosphate, estolate, fumarate, gluconate, malate, maleate, pamoate, stearate, succinate, sulfate, sulfonate, tartrate, tosylate, and valerate.

38. The compound of claim 37, wherein the counter ion is phosphate or hydrochloride.

39. The compound of any one of claims 1 to 38, which has a greater solubility in water than rimiducid.

40. The compound of any one of claims 1 to 39, which has a solubility greater than 1 mg·mL⁻¹ in water.

41. The compound of any one of claims 1 to 39, which has a solubility greater than 2.5 mg·mL⁻¹ in water.

42. The compound of any one of claims 1 to 39, which has a solubility greater than 0.2 mg·mL⁻¹ in acetate buffer or phosphate buffer having a pH of 6 or less.

43. A pharmaceutical composition comprising a compound of any one of claims 1 to 42, and a pharmaceutically acceptable excipient or carrier.

44. The pharmaceutical composition of claim 43, which is a liquid composition.

45. The pharmaceutical composition of claim 44, which is 80% or greater (weight/weight) water.

46. A method of multimerizing polypeptides expressed in a cell, comprising contacting the cell with a compound of any one of claims 1 to 42, or with a pharmaceutical composition of any one of claims 43 to 45, wherein the polypeptides each comprise one or more FKBP12v36 polypeptides.
